(19) United States
(12) Patent Application Publication
(10) Pub. No.: US 2022/0199245 A1
(43) Pub. Date: Jun. 23, 2022

(54) SYSTEMS AND METHODS FOR SIGNAL BASED FEATURE ANALYSIS TO DETERMINE CLINICAL OUTCOMES

(71) Applicant: Regeneron Pharmaceuticals, Inc., Tarrytown, NY (US)

(72) Inventors: Matthew F. WIPPERMAN, Brooklyn, NY (US); Xuefang WU, Hopkinton, MA (US); Yiziying CHEN, White Plains, NY (US); Rinol ALAJ, Jersey City, NJ (US); Sara HAMON, Darien, CT (US); Olivier HARARI, New York, NY (US)

(73) Assignee: Regeneron Pharmaceuticals, Inc., Tarrytown, NY (US)

(21) Appl. No.: 17/645,660

(22) Filed: Dec. 22, 2021

Related U.S. Application Data
(60) Provisional application No. 63/129,357, filed on Dec. 22, 2020.

(51) Int. Cl. G16H 40/63; G16H 20/40; G16H 50/20

(52) U.S. Cl. CPC G16H 40/63; G16H 50/20; G16H 20/40

(57) ABSTRACT

The present disclosure provides methods for receiving distinct electrical signals generated based on a body part, generating a plurality of extracted features based on the distinct electrical signals, identify clinically relevant features from the plurality of extracted features, wherein the clinically relevant features meet a threshold determined based on a clinical outcome.

### CROSS-REFERENCE TO RELATED APPLICATIONS

This application claims priority to U.S. Provisional Application No. 63/129,357, filed on Dec. 22, 2020, the entirety of which is incorporated by reference herein.

### TECHNICAL FIELD

Embodiments disclosed herein are directed to systems and methods for profiling features derived from signals (e.g., signals based on biometric cues in a subject using a biometric device, including, but not limited to wearable devices) for use in clinical outcomes. The clinical outcomes may include early detection and/or treatment of potential disease or disorder experienced by a patient. Aspects of an example wearable biometric device are also disclosed.

### INTRODUCTION

Identifying statistical data for providing clinical outcomes (e.g., for clinical trials, for disease or disorder identification, for treatment planning, etc.) is difficult due to the type, volume, and/or depth of available data.

For example, various neuromuscular disorders affect nerves carrying electrical signals that control voluntary muscles. The disorders impair and can progressively debilitate the nerves, causing the muscles to atrophy and die over time. One example of a neuromuscular disorder is Myasthenia Gravis (MG), which causes facial affects, such as, drooping eyelids (ptosis), double vision (diplopia), and/or difficulty making facial expressions. Additionally, Myasthenia Gravis (MG) can cause difficulty talking, breathing, chewing, and/or swallowing. Traditionally, medical professionals screen patients for neuromuscular disorders via observation and self-assessments (e.g., patient-reported outcomes). For example, an evaluation may involve a clinician performing a questionnaire scoring the patient's observed physical affects (e.g., ptosis and gaze) and ability to perform certain activities (e.g., eye closure, talking, and chewing). Such methods are inaccurate because the observations are subjective and because patients may adapt their behaviors over time to compensate for problematic symptoms. In clinical visits, patients often under-report chewing and swallowing symptoms and severity grades due to patients adapting to softer and liquid dietary habits especially when the patients are having the symptoms for a long time. Consequently, the current assessment methods that have been using in clinic may lead to incorrect and missed diagnoses and treatments. As such, there is an unmet medical need to accurately assess the symptoms and severity grades in patients objectively and quantitatively.

Use of statistical data to generate diagnoses and treatment may provide results based on objective data. However, use of statistical data is challenging due to the type, volume, and/or depth of available data for a given trial. For example, data applicable to identify one clinical outcome may not apply to identifying another clinical outcome. Additionally, a given data parameter, signal collection mechanism, and/or action during signal collection may be optimal for a first clinical output yet may not be optimal for a second clinical output.

Accordingly, there is a need for improved techniques for making assessment, determining diagnoses, and assigning treatments to patients with neuromuscular disorders.

### SUMMARY OF THE DISCLOSURE

Aspects of the present disclosure relate to signal based feature analysis. In one aspect, the present disclosure is directed to a method including receiving distinct electrical signals generated based on a body part, generating a plurality of extracted features based on the distinct electrical signals, and identify clinically relevant features from the plurality of extracted features, wherein the clinically relevant features meet a threshold determined based on a clinical outcome.

The method may also include applying the clinically relevant features to determine a clinical outcome result, wherein the clinical outcome result is one of a diagnosis or a treatment plan. The distinct electrical signals may be generated based on a body electrical signal generated by the body part. The distinct electrical signals may be generated based on a movement of the body part. The distinct electrical signals may be generated based on a property of the body part. The plurality of extracted features may be based on one or more of amplitude features, zero crossing rate, standard deviation, variance, root mean square, kurtosis, frequency, bandpower, or skew. The distinct electrical signals may be generated by a wearable device comprising sensors, wherein the wearable device may be configured to output a mixed signal and/or wherein a signal separation module extracts the extracted features from the mixed signal.

For example, the signal separation module may apply one or more of blind signal separation, blind source separation, discrete transform, Fourier transform, integral transform, two-sided Laplace transform, Mellin transform, Hartley transform, Short-time Fourier transform (or short-term Fourier transform) (STFT), rectangular mask short-time Fourier transform, Chirplet transform, Fractional Fourier transform (FRFT), Hankel transform, Fourier-Bros-Iagolnitzer transform, or linear canonical transform to extract the extracted features from the mixed signal. A random forest algorithm may be used to score the extracted features. The threshold may be a random forest threshold and extracted features having a random forest score at or above the random forest threshold may be identified as clinically relevant features. The threshold may be a reliability threshold and extracted features having a reliability score at or above a reliability threshold may be identified as clinically relevant features. The reliability score may be based on one or more of a spearman correlation, intraclass correlation (ICC), covariance (CV), area under a curve (AUC), clustering, or Z score.

In another aspect, the present disclosure is direct to a system including a wearable device including a plurality of sensors, a processor, a computer-readable data storage device storing instructions that, when executed by the processor, cause the system to obtain electrical activity information of a subject from the wearable device, the electrical activity detected by the plurality of sensors, and identify clinically relevant features based on the electrical activity information.

The system may be further configured to classify the clinically relevant features as one or more maladies, determine a disease of the subject based on the one or more maladies, determine a scope of the disease and/or determine a treatment plan based on the scope of the disease. The plurality of sensors may include an electroencephalography (EEG) sensor, an electrooculography (EOG) sensor, an electromyography (EMG) sensor, an image sensor, and/or an eye-tracking sensor. The clinically relevant features may be identified using a machine-learning algorithm.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIG. 1A is a relationship diagram illustrating feature extraction and selection, in accordance with aspects of the present disclosure.

FIG. 1B is a system block diagram illustrating an example headgear, in accordance with aspects of the present disclosure.

FIG. 2 is a system block diagram illustrating an example of an environment for implementing systems and processes, in accordance with aspects of the present disclosure.

FIG. 3 is a block diagram illustrating an example of a controller, in accordance with aspects of the present disclosure.

FIGS. 4A and 4B are flow diagrams illustrating an example of a method performed by a system, in accordance with aspects of the present disclosure.

FIG. 4C is a flowchart illustrating identification and application of clinically relevant features, in accordance with aspects of the present disclosure.

FIG. 5 shows the limitations of existing clinical outcome measures as some are subject to recall bias.

FIG. 6 shows exemplary output readings, according to an embodiment of the present disclosure.

FIG. 7 shows additional exemplary output readings, according to an aspect of the present disclosure.

FIG. 8 is a heat map of four (4) tasks and z score correlation, according to an embodiment of the present disclosure.

FIG. 9 shows a heat map of variables and activities that demonstrates qualitative inter subject and inter activity differences, according to an embodiment of the present disclosure.

FIG. 10 shows a Spearman plot of the data from FIG. 9 evidencing parameters that are highly correlated are likely measuring similar aspects of facial biology, according to an embodiment of the present disclosure.

FIG. 11 shows intraclass correlation (ICC) measures that test re-test reliability of parameters and infer clinical significance, according to an embodiment of the present disclosure.

FIG. 12 also shows ICC measures that test re-test reliability of parameters and helps infer clinical significance, according to an embodiment of the present disclosure.

FIG. 13 shows a schema of a Random Forest approach, according to an embodiment of the present disclosure.

FIG. 14 shows F1 score(s) to measure how well a model classifies a particular activity (e.g., swallowing), according to an embodiment of the present disclosure

FIG. 15 shows improved F1 scores using the parameters from two rounds of feature engineering, resulting in improvement F1 score for some activities, according to an embodiment of the present disclosure.

FIG. 16 shows improved F1 scores presented in a histogram format, according to an embodiment of the present disclosure.

FIG. 17 shows F1 scores in the morning and evening, according to an embodiment of the present disclosure.

FIG. 18 shows as chart for algorithm options for selection, according to an embodiment of the present disclosure.

FIG. 19 shows swallowing values for different bandwidths, according to an embodiment of the present disclosure.

FIG. 20 shows clusters and corresponding channels, according to an embodiment of the present disclosure.

FIG. 21 shows amplitude, frequency, and band-power channels with other factors, according to an embodiment of the present disclosure.

FIG. 22 shows the covariance (CV) for a mixed effect model based on morning measurements, according to an embodiment of the present disclosure.

FIG. 23 shows measurement results of various components collected during multiple measurement times, according to an embodiment of the present disclosure.

FIG. 24 shows Z scores for various combinations of individuals, time, and tasks, according to an embodiment of the present disclosure.

FIG. 25 shows tasks plotted on a Uniform Manifold Approximation and Projection (UMAP) chart, according to an embodiment of the present disclosure.

FIG. 26 shows individual data plotted on a Uniform Manifold Approximation and Projection (UMAP) chart, according to an embodiment of the present disclosure.

FIG. 27 shows time data plotted on a Uniform Manifold Approximation and Projection (UMAP) chart, according to an embodiment of the present disclosure.

FIG. 28 shows swallowing values over multiple channels, according to an embodiment of the present disclosure.

FIG. 29 shows random forest importance values across multiple channels, according to an embodiment of the present disclosure.

FIG. 30 shows cluster statistics, according to an embodiment of the present disclosure.

FIG. 31 shows Z scores across tasks during morning collections, according to an embodiment of the present disclosure.

FIG. 32 shows Z scores across tasks during evening collections, according to an embodiment of the present disclosure.

FIG. 33 shows Z scores for Leave-one-out cross-validation (LOOCV) across tasks, according to an embodiment of the present disclosure.

FIG. 34 shows Z scores for standard deviation (SD) during morning collections, according to an embodiment of the present disclosure.

FIG. 35 shows Z scores across tasks during evening collections, according to an embodiment of the present disclosure.

FIG. 36 shows CV for a mixed effect model for evening collections, according to an embodiment of the present disclosure.

FIG. 37 shows band-power measurements spreads for smile collections, according to an embodiment of the present disclosure.

FIG. 38 shows a cluster ICC heat map for evening measurements, according to an embodiment of the present disclosure.

FIG. 39 shows a cluster ICC heat map for a mixed effect model with evening measurements, according to an embodiment of the present disclosure.

FIG. 40 shows t-distributed stochastic neighbor embedding (t-SNE) charts for individuals, tasks, and time, according to an embodiment of the present disclosure.

FIG. 41 shows various charts for collection classes, according to an embodiment of the present disclosure.

FIG. 42 shows UMAP charts for individuals, tasks, and time, according to an embodiment of the present disclosure.

FIGS. 43 and 44 show confirmation results for different tasks across various channels, according to an embodiment of the present disclosure.

FIG. 45 is a flow diagram of a feature engineering process, according to an embodiment of the present disclosure.

FIG. 46 shows feature representation in the frequency and time domain, according to an embodiment of the present disclosure.

FIG. 47 shows qualitative differences observed from representative signals, according to an embodiment of the present disclosure.

FIG. 48 shows a spearman correlation of features, according to an embodiment of the present disclosure.

FIG. 49 shows Qualitative differences between the 16 mock-PerfO activities, according to an embodiment of the present disclosure.

FIG. 50 shows heat maps of features, according to an embodiment of the present disclosure.

FIG. 51 shows CNN models built with biometric sensor device raw bio-signal data, according to an embodiment of the present disclosure.

FIG. 52 shows an activity chart for activities with level classification F1 scores for biometric sensor device features, according to an embodiment of the present disclosure.

FIG. 53 shows a heat map of feature attribution analysis, according to an embodiment of the present disclosure.

FIG. 54 is a data flow for training a machine learning model, according to one or more embodiments.

FIG. 55 is an example diagram of a computing device, according to one or more embodiments.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. In the discussion that follows, relative terms such as “about,” “substantially,” “approximately,” etc. are used to indicate a possible variation of ±10% in a stated numeric value.

Aspects of the disclosed subject matter are generally directed to receiving signals generated based on a body component of an individual. The signals may be or may be generated based on electrical activity, physical activity, biometric data, movement data, or any attribute of an individual's body, an action associated with the individual's body, reaction of the individual's body, or the like. The signals may be generated by a signal capture device that may capture the signals using one or more sensors. For example, aspects of the disclosed subject matter are directed to methods for profiling biometric cues in a subject using a wearable biometric device. Computer mediated profiling for the early detection of a potential disease or disorder in a patient is also described such that an early diagnosis can be obtained, and a therapy implemented. Aspects of an example wearable biometric device are also disclosed.

Implementations of the disclosed subject matter include a wearable system for identifying biometric cues in human subjects. Systems and techniques disclosed herein may be used to resolve unacceptable detection and treatment gaps in patients presenting with a neurological disease or disorder. In particular, a noninvasive wearable biometric device (e.g., a behind the ear device) is disclosed to detect patient movements, in particular, facial movements such as talking, chewing, swallowing, neck movements, and/or eye movements.

Implementations of the disclosed subject matter provide ways of uploading large amount of data for analysis. For example, the analysis may be performed using sophisticated statistical analysis and machine based learning (or artificial intelligence), so reliable results can be secured, retested, and understood. Systems and techniques disclosed herein allow for patient comfort and compliance, a large array of input/output channels for large data harvesting, machine assisted statistical analyses with high reliability, early detection of disorders or diseases, early intervention for the same, and improved clinical outcomes.

Implementations of the disclosed subject matter may be used to detect disease and/or disorder based on collection of objective statistical data. For example, implementations of the disclosed subject matter may be suitable for neurological disease, for example, Myasthenia Gravis (MG), where subclinical cues can go undetected.

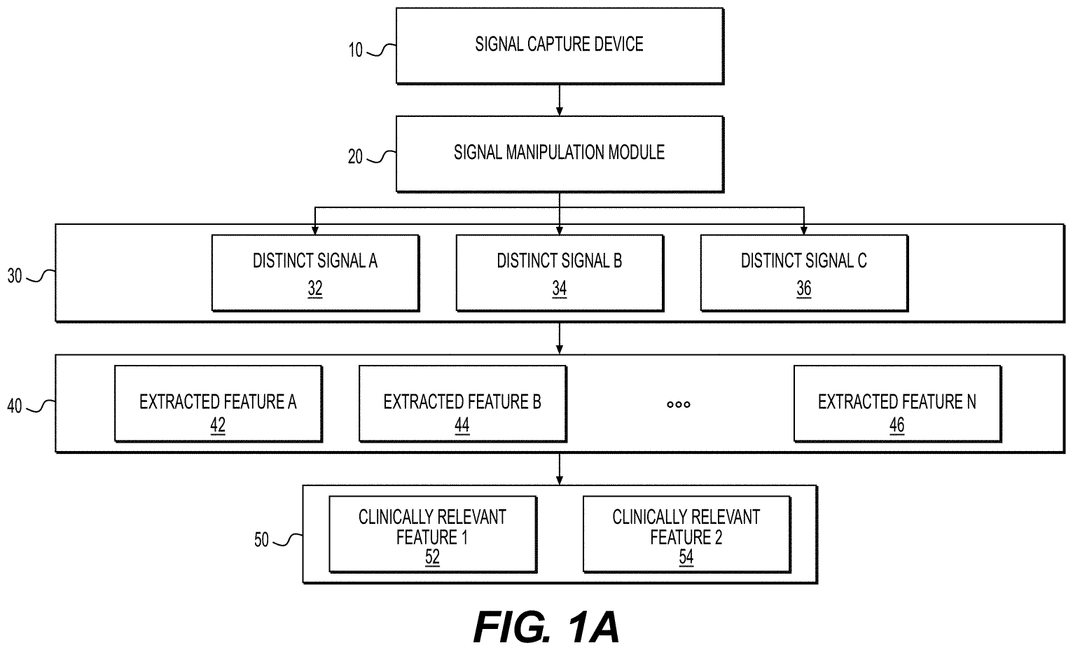

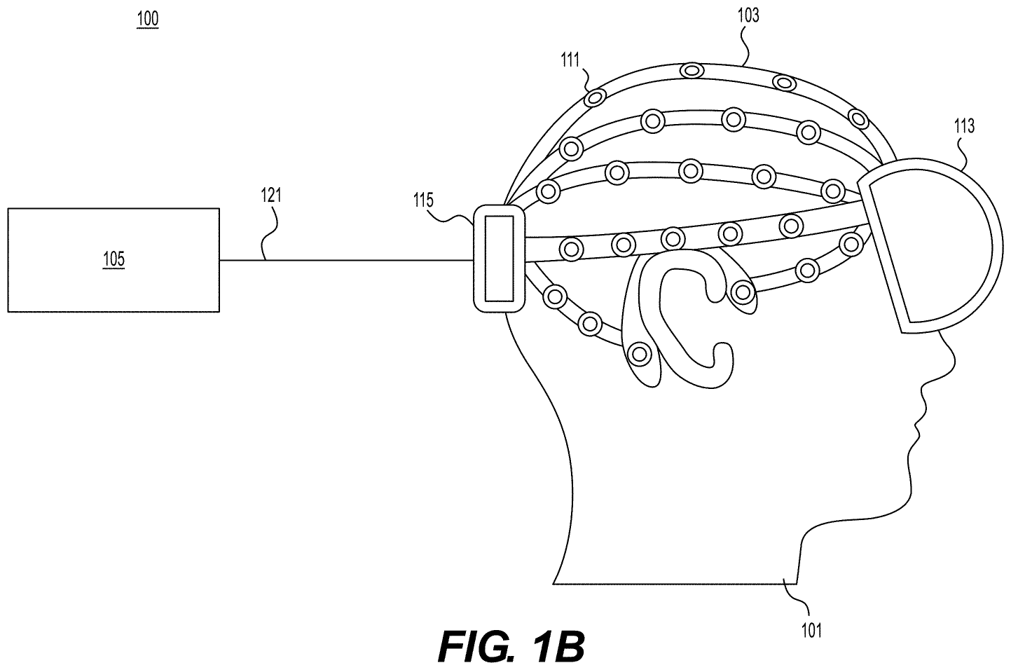

Improving pipelines for the development and analysis of wearable sensor data and frameworks for how to use these data in clinical settings is critical for improving accurate patient diagnosis and monitoring treatment responses in all stages of clinical drug development. There exists challenges in both the development of wearable devices as well the ways in which wearable data are processed and analyzed in clinical settings. Techniques disclosed herein address these issues and include example proof-of-concepts using signal capture devices 10 (e.g., a sleep aid/biometric sensor wearable that measures electromyography (EMG), electroencephalography (EEG), and electrooculography (EOG)), as shown in FIG. 1B, to assess facial and/or ocular muscle movements in a study of healthy controls. The utility of an unbiased feature engineering approach to classify activities intended to be representative of true Performance Outcome Assessments (PerfOs) is disclosed. An approach for analyzing and ranking the utility of features generated from biometric sensor devices and how this data may be used to classify activities performed in this study setting is disclosed. Limitations of time series analysis on bio-signal data collected over short periods of time compared to a feature-based analytical approach is also disclosed.

Data generated by biometric sensor devices can be used to classify an individual's body information (e.g., certain types of cranial muscle and ocular movements). The data disclosed herein suggests that biometric wearable devices can be used to objectively monitor certain body information (e.g., cranial movements, such as eye blinking rate). For example, such body information may include movement which is increased in some neuromuscular disorders such as ocular myasthenia gravis and reduced in parkinsonian disorders. Additionally, there are advantages of measuring multiple types of waveforms simultaneously from a single device, given the demonstrated utility of these waveforms to measure disease in clinical settings.

As disclosed herein, feature importance analyses can indicate that EOG is associated with contributing largely to gaze or eye movement activities (up, left, and right) when analyzing which features are most important at classifying activities using the RF model. EOG, EMG, and other signals play an important role in such indications. The presence of signal artifacts may obfuscate waveform contribution analysis. For example, when performing a chewing activity, where residual EMG activity that overlapped with typical EEG frequencies persisted in the EEG signal after signal separation, an overestimate of EEG waveform contribution resulted. There are numerous neuromuscular and/or neurodegenerative conditions that may benefit from improved use of wearable sensor technology, as discussed herein.

Techniques disclosed herein include several feature engineering and evaluation considerations. Classification accuracy (F1 scores) of models built from both processed sensor data are compared, as well as models built from raw bio-signal data. Regardless of data augmentation, regularization, and other techniques used to counter overfitting, training dataset used in examples provided herein was observed to be too small to train a generalizable Convolutional Neural Network (CNN) model. However, the level or amount of data collected in the examples disclosed herein may be representative of data collected in a clinical laboratory setting. As such, as further disclosed herein, understanding the most appropriate analysis method (e.g., clinically relevant features) for a particular clinical outcome is important. The analysis method (e.g., clinically relevant features) used for a given clinical outcome may be different from another clinical outcome.

The term “algorithm” refers to a sequence of defined computer-implementable instructions, typically to solve a class of problems or to perform a computation. FIGS. 1-3, 4A, and 4B provide components for implementation of algorithms and/or examples of algorithms.

The term “AUC” refers to the Area Under the Curve, as understood in the art, related to statistical analysis.

The term “BCI” refers to a Brain Computer Interface system that measures activity of the central nervous system (CNS) and converts the activity into artificial and/or digital outputs for analysis.

The term “BMI” refers to Body Mass Index value derived from the mass and height of a person. The BMI is a recognized metric to broadly categorize a person as underweight, normal weight, and overweight. BMI is frequently measured as a factor for entry into a clinical trial.

The term “CNN” refers to a Convolutional Neural Network algorithm which can receive an input, assign importance (learnable weights and biases) to various aspects/objects in the input, and be able to differentiate between the various aspects/objects. A CNN may use deep learning to perform both generative and descriptive tasks, often using machine vison that includes image and video recognition, along with recommender systems and natural language processing (NLP). The layers of a CNN may include of an input layer, an output layer, and a hidden layer that includes multiple convolutional layers, pooling layers, fully connected layers and normalization layers. The removal of limitations and increase in efficiency for processing results in a system that is far more effective and is simpler to train.

The term “CV” refers to covariance in statistics, wherein a positive number is output if variables being measured are positively related and a negative number is output if they are negatively related. A high covariance may indicate that there is a strong relationship between the variables. A low value may indicate that there is a weak relationship between the variables.

The term “EEG” refers to electroencephalography, the biometric evaluation of brain activity. An EEG may detect abnormalities in brain waves, or in the electrical activity of a brain. An EEG may be collected by using electrodes having small metal discs with thin wires are pasted onto you're a scalp. The electrodes may detect electrical charges that result from the activity of brain cells. As disclosed herein, EEG data may be detected using a non-invasive device (e.g., a behind the ear device).

The term “EMG” refers to electromyography, the biometric evaluation of facial muscle weakness. EMG data may be collected by recording or receiving the electrical activity of muscle tissue, or its representation as a visual display or audible signal, using electrodes attached to the skin or inserted into the muscle. As disclosed herein, EMG data may be detected using a non-invasive device (e.g., a behind the ear device).

The term “EOG” refers to electrooculography, the evaluation of eye movement activity. EOG data may be collected by measurement of the electrical potential between points close to the eye, used to investigate eye movements especially in physiological research. As disclosed herein, an EOG data may be detected using a non-invasive device (e.g., a behind the ear device).

The term “F1 score” refers to a measure of a model's accuracy on a dataset as a binary classification wherein a score of 0 is poor and a score of 1 is best. The F1 score may be calculated from the precision and recall of the test, where the precision is the number of true positive results divided by the number of all positive results, including those not identified correctly, and the recall is the number of true positive results divided by the number of all samples that should have been identified as positive. Precision may be the positive predictive value, and recall may be a sensitivity in diagnostic binary classification. The F1 score may be a harmonic mean of the precision and recall.

The term “false positive” refers to an outcome where a model incorrectly predicts the positive class.

The term “false negative” refers to an outcome where the model incorrectly predicts the negative class.

The term “ICC” refers to intraclass correlation coefficient that can be used when quantitative measurements are made on units that are organized into groups. It can be used to evaluate the consistency or reproducibility of quantitative measurements made by different observers measuring the same quantity.

The term “ISO” refers to an isometric measure relating to or denoting muscular action in which tension is developed without contraction of the muscle.

The term “LOOCV” refers to Leave One Out Cross Validation Analysis, a procedure used to estimate the performance of machine learning algorithms. In LOOCV, a number of folds may equal the number of instances in a data set. Thus, the learning algorithm may be applied once for each instance, using all other instances as a training set and using the selected instance as a single-item test set.

The term “MG” refers to Myasthenia Gravis, a neurodegenerative disease that can be evaluated by the application of the disclosed subject matter.

The term “PSG” refers to polysomnography, a type of sleep study using multi-parametric tests as a diagnostic tool in sleep medicine. During a PSG analysis, brain waves, oxygen level in blood, heart rate, breathing, as well as eye and leg movements may be recorded and/or analyzed.

The term “Random Forest” refers to combining many decision trees into a single model. Individually, predictions made by decision trees (or humans) may not be accurate, but a combination of such predictions may increase their overall accuracy. Random forests or random decision forests are an ensemble learning method for classification, regression and other tasks that operates by constructing a multitude of decision trees at training time. For classification tasks, the output of the random forest may be the class selected by most trees.

The term “RMS” refers to root mean square, a statistical measure of the magnitude of a varying quantity. RMS can be calculated for a series of discrete values or for a continuously varying function.

The term “SD” refers to standard of deviation. A low standard of deviation indicates that the values tend to be close to the mean (also called the expected value) of the set, while a high standard deviation indicates that the values are spread out over a wider range.

The term “spectrogram” refers to a visual representation of the spectrum of frequencies of a signal as it varies with time.

A term “true positive” refers to an outcome wherein a model correctly predicts the positive class.

The term “true negative” refers to an outcome wherein the model correctly predicts the negative class.

The term “Z score” refers to a value of how many standard deviations given data is away from the mean. If a Z score is equal to 0, the data is at the mean. A positive Z score indicates that a raw score is higher than the mean average. A negative Z score indicates that a raw score is below the mean average.

A review of classification techniques of EMG signals during isotonic and isometric contractions; Sensors. 2016 Aug. 17; 16(8):1304; Real-Time Surface EMG Pattern Recognition for Hand Gestures Based on an Artificial Neural Network. Sensors. 2019 July; 19(14): 3170; and Techniques of EMG signal analysis: detection, processing, classification, and applications. Biol. Proceedings. Online. 2006; 8: 11-35, are each incorporated by reference and are relevant to EMG and/or EOG.

Harpale, V. K. and Vinayak K. Bairagi. “Time and frequency domain analysis of EEG signals for seizure detection: A review.” 2016 International Conference on Microelectronics, Computing and Communications (MicroCom) (2016): 1-6 is incorporated by reference and is relevant to algorithms as discussed herein.

Challenges in Detecting Physiological Changes Using Wearable Sensor Data (SciPy 2019), is incorporated by reference and is relevant to interpretation of Time Series Data.

WO2016110804A1 is incorporated by reference herein and describes exemplary mobile wearable monitoring systems, including headgear devices, used in connection with the principles of the present disclosure.

According to implementations of the disclosed subject matter, as shown in FIG. 1A, a signature capture device 10 may be used to capture signals associated with an individual's body. The signals may be based on a body's electrical activity, physical movement, biometric information, temperature information, actions, reactions, or any attribute that can be captured as a signal (e.g., as an electrical signal). Signal capture device 10 may include one or more sensors, electrodes, cameras, or other components used to capture a signal based on an individual's body. Signals captured by signal capture device 10 may include one or more distinct signals 30 (e.g., distinct signal A 32, distinct signal B 34, and distinct signal C 36).

Alternatively, signals captured by signal capture device 10 may be processed through a signal manipulation module 20. Signal manipulation module 20 may apply signal filtering techniques to parse distinct signals 30 from the raw data received from signal capture device 10. For example, signal manipulation module 20 may apply one or more of blind signal separation, blind source separation, discrete transform, Fourier transform, integral transform, two-sided Laplace transform, Mellin transform, Hartley transform, Short-time Fourier transform (or short-term Fourier transform) (STFT), rectangular mask short-time Fourier transform, Chirplet transform, Fractional Fourier transform (FRFT), Hankel transform, Fourier-Bros-Iagolnitzer transform, linear canonical transform, and/or the like to identify distinct signals 30 from the signals provided by signal capture device 10.

Distinct signals 30 may be used to generate a plurality of extracted features 40 (e.g., extracted feature A 42, extracted feature B 44 . . . extracted feature N 46). Extracted features 40 may be generated based on properties of the extracted features 40 either alone or in combination with each other. For example, extracted features 40 may be based on one or more of signal frequency bandpowers (e.g., for each of the distinct signals 30 across multiple frequencies), spectral entropy, peak frequency contractions, peak frequency, mean amplitudes, percentage absolute amplitudes, standard deviation, absolute amplitude standard deviations, root mean squares, initial deflection max amplitudes, initial deflection polarities, detrended fluctuation hurst parameters, petrosian fractal dimensions, approximate entropies, zero crossing rates, amplitude kurtosis, amplitude skews, perceptible onset times, amplitude variances, and/or any applicable signal based attribute.

Implementations disclosed herein may be used to identify clinically relevant features 50 (e.g., clinically relevant feature 52 and/or clinically relevant features 54) for a given clinical output, based on the extracted features 40. For example, a first set of extracted features may be optimal for identifying a first clinical output (e.g., a disease or disorder diagnosis or treatment) whereas a second set of extracted features may not be optimal for identifying the first clinical output but may be optimal for identifying a second clinical output. Accordingly, techniques disclosed herein may be used to identify clinically relevant features 50 for a given clinical output based on one or more of extracted features 40, distinct signals 30, signal manipulation module 20, signal capture device 10, the clinical output, an individual, or the like. For example, signal capture device 10 may have a sensitivity quality to generate a first feature well but may not be sufficiently sensitive to generate a second feature. Accordingly, when using signal capture device 10, the first feature may be identified as a clinically relevant feature whereas the second feature may not be identified as a clinically relevant feature.

Similarly, a given clinical outcome (e.g., diagnosis of Parkinson's disease) may be identified using a first feature that has a low standard deviation across individuals with Parkinson's disease when compared to a second feature that has a higher standard deviation across individuals with Parkinson's' disease. Accordingly, the first feature may be identified as a clinically relevant feature as it may be more consistent in, for example, predicting the presence of Parkinson's when compared to the second feature.

Clinically relevant features 50 may be identified based on signal data collected and analyzed for a test user or cohort of test users. A cohort of test users may be any group of test users or a group of test users that has an attribute (e.g., a demographic attribute) that overlaps with one or more individuals. For example, one or more signal capture devices 10 may be used to generate distinct signals 30 for one or more test users. Extracted features 40 may be generated from these distinct signals 30. Clinically relevant features 50 may be identified for each individual and for a given clinical output (e.g., detection of a disorder). As disclosed herein, these clinically relevant features 50 may meet or exceed one or more reliability thresholds such that the clinically relevant features 50 can be relied upon to produce the clinical output with a degree of confidence. Clinically relevant features 50 identified based on data from one or a cohort of test users may be authorized for clinical trial use based on a clinical output degree of confidence. One or more individuals may participate in such a clinical trial such that the data corresponding to the clinically relevant features 50 for those one or more individuals may be compared to reference data (e.g., data from the one or a cohort of test users).

Additionally, or alternatively, data obtained for a given individual and/or combined data for a plurality of individuals may be used as an endpoint in a given trial. For example, the identification of clinically relevant factors 50 may be the end point in a clinical trial. The endpoint may provide an indication of the quality and/or capabilities of a signal capture device 10 being tested in the clinical trial. The endpoint may alternatively provide an indication of the clinically relevant factors 50 that reliably provide a clinical output given a patient population, type of disease or disorder, and/or signal data.

Additionally, or alternatively, data (e.g., signal data, discrete signals 30, extracted features 40, and/or clinically relevant features 50) from an individual may be compared to corresponding data from one or more other individuals. For example, such data may be collected from each of a plurality of users in a clinical trial. In this example, the data for one or more individuals receiving a treatment (e.g., a drug, a therapy, etc.) may be compared to respective data for one or more individuals receiving an alternative treatment (e.g., a different dosage, duration, or type of drug or therapy), receiving no treatment (e.g., a placebo group), and/or to a reference set of data (e.g., control data). According to an implementation, the data associated with a given individual at a first time may be compared to data from that individual at a second time.

The comparison of data (e.g., signal data, discrete signals 30, extracted features 40, and/or clinically relevant features 50) from different individuals (e.g., receiving different treatment, placebo, control group, etc.), or the same individual over time, may be used to identify a clinically relevant factor. A clinically relevant factor may be, for example, the effect of a given treatment, the effect of a dosage or amount of time of a treatment, the differences in the presentation a given disease or disorder in different individuals (e.g., for optimal treatment planning), identification of a cluster or grouping, and/or the like.

According to an example implementation disclosed herein, electrical information of an individual's brain, nervous system, and/or muscles may be obtained. Additionally, or alternatively, sensory (e.g., visual) information (e.g., videos, images, infra-red images, heat images, vibrations, etc.) of an individual's body (e.g., the individual's face or parts thereof) may be obtained. According to implementations, a headgear or a wearable device (e.g., signal capture device 10) may be used to collect signals from an individual's body. The headgear or wearable device may include one or more sensors to capture the electrical information, and/or sensory information. It will be understood that although terms headgear and/or wearable device are used herein, any instrument that is configured to capture electrical or sensory information at a body part (e.g., the individual's face or parts thereof) may be used in accordance with the techniques disclosed herein. Headgear and/or wearable device may include any devices configured to rest and/or be placed at or around an individual's head or body part. Headgear and/or wearable device may be secured or unsecured to a portion of an individual's body.

According to an example implementation, a headgear and/or wearable device may include sensors for capturing electrical signals. Such electrical signals may include electroencephalography (EEG) data, electrooculography (EOG) data, and/or electromyography (EMG) data. Also, an example headgear and/or wearable device may include sensory information sensors (e.g., image sensors, video sensors, infra-red sensors, heat sensors, vibration sensors, etc.) for capturing individual input data such as facial data (e.g., facial recognition data), eye-tracking data, movement data, environmental data (e.g., heat data), or the like. Further, a controller may receive signal data (e.g., EEG, EOG, EMG, as well as the individual input data).

The controller may be configured to classify the signal data (e.g., EEG, EOG, EMG, and individual input data) and that signal data may be used to identify a clinical outcome. For example, the signal data may be used to identify whether an individual has one or more maladies. Alternatively, or in addition, the signal data may be used to determine properties of an individual's maladies to provide a treatment plan for the individual. According to implementations disclosed herein, a classification may be performed using machine learning techniques. Moreover, in some implementations, the systems and methods may further combine the signal data (e.g., EEG, EOG, EMG data, and the individual input data) into a classification of a potential condition.

Techniques disclosed herein may be used to determine a scope of the condition and/or a treatment plan corresponding to the scope. According to implementations of the disclosed subject matter, wearable biometric devices (e.g., headgear and/or wearable device) may be used for detecting and/or treating a disease or disorder in an individual. The disclosed subject matter can be used for early identification of a disease or disorder that may be preclinical in its presentation, silent, and/or undiagnosed. The techniques disclosed herein have wide application for quantifying a range of neurological and muscular diseases and disorders.

According to implantation of the disclosed subject matter, techniques in the field of patient intake, statistical analysis, use of wearable electronics, artificial intelligent (AI), algorithms, machine based learning, statistical analysis, and wearable devices and/or electronic modes of measuring a human subject may be implemented for securing unbiased objective metrics related to an individual and/or a disease or disorder. Objective data driven analysis may be used to remove inaccurate patient self-reporting and potential statistical noise to achieve reliable metrics to proactively identify human disease or disorders. Though such information bay be supplemented with subjective inquiries during intake and/or to determine baseline, objective analysis may be used to more accurately identify a disease or disorder and/or to provide a treatment plan.

According to implementations of the disclosed subject matter, high reliability measurement of biometric cues in an individual may be used to detect and/or treat diseases or disorders. One or more sensors (e.g., placed at or about headgear) may be used to collect biometric measurements of an individual at a given point in time and/or over a range of time. FIG. 1B shows a block diagram illustrating an example of an environment 100 for implementing systems and methods in accordance with aspects of the present disclosure. In some implementations, environment 100 can include an individual 101, headgear 103, and a controller 105. Headgear 103 may correspond to signal capture device 10 of FIG. 1A.

Individual 101 can be any person. In some implementations, individual 101 may be a baseline individual for collection of control data. In some implementations, individual 101 may be a medical patient. For example, individual 101 may be a patient that may have a neuromuscular disorder, such as Myasthenia Gravis. It will be understood that though “headgear” and “wearable device” is generally referenced herein, a device for collection of electrical and/or individual input data may be positioned above, below, around, partially around, or in any applicable position near an individual's head or other body part. For example, headgear 103 may refer to eyeglasses that may rest on an individual's ears. As another example, headgear 103 may refer to ear pieces that are inserted in or around an individual's ear.

Headgear 103 may be a device including one or more sensors that capture information of individual 101 representing the operation of voluntary muscles. The sensors may collect electrical information and/or individual input information (e.g., facial data (e.g., facial recognition data), eye-tracking data, movement data, environmental data (e.g., heat data), or the like.) In some implementations, headgear 103 may include electrical sensors 111, sensory information sensors 113, and/or a device controller 115. Electrical sensors 111 may be configured to capture EEG data, EOG data, and EMG data. Sensory information sensors 113 may include facial recognition sensors, eye tracking sensors, image sensors, video sensors, infra-red sensors, heat sensors, vibration sensors.

Device controller 115 may be a computing device connected to the controller 105 through one or more wired or wireless communication channels 121. Communication channels 121 may use various serial, parallel, and/or transmission (e.g., video transmission) protocols. Device controller 115 may include hardware, software, firmware, or a combination thereof for performing operations in accordance with the present disclosure. The operations may include receiving the EEG, EOG, EMG, individual input data such as facial data (e.g., facial recognition data), eye-tracking data, movement data, environmental data (e.g., heat data), or the like from electrical sensors 111 and/or sensory information sensors 113 and transmitting data to the controller 105 using the communication channel 12,1 using one or more transmission protocols.

Controller 105 may include hardware, software, or a combination thereof for performing operations in accordance with the present disclosure. Operations performed by controller 105 may include receiving, filtering, and normalizing the data transmitted by device controller 115 of headgear 103. The operations may also include individually classifying the EEG, EOG, EMG, and/or individual input data by determining one or more descriptive categories and respective severities. The categories may be species or genera of symptoms or disorders. In some implementations, the classification can be performed using machine learning techniques. For example, an elaborate array of sophisticated statistical data may be interpreted using a random forest schema, as further discussed herein.

Implementations of the disclosed subject matter include classifying a combination the EEG, EOG, EMG, and individual input information by determining one or more descriptive categories or disorders. In some implementations, the classification can be performed using machine learning techniques to classify the symptoms or disorders determined using the individual classifications of the data. In some implementations, the operations further include determining a treatment plan corresponding the one or more disorders and their respective severities.

For example, FIG. 1B shows a system diagram illustrating an example of the headgear 103 in accordance with aspects of the present disclosure. The headgear 103 can be the same or similar to the headgear and/or wearable device discussed above. In some implementations, the headgear 103 can include a device controller 115, electrical sensors 111, and sensory information sensors 113, which can be the same or similar to those previously described.

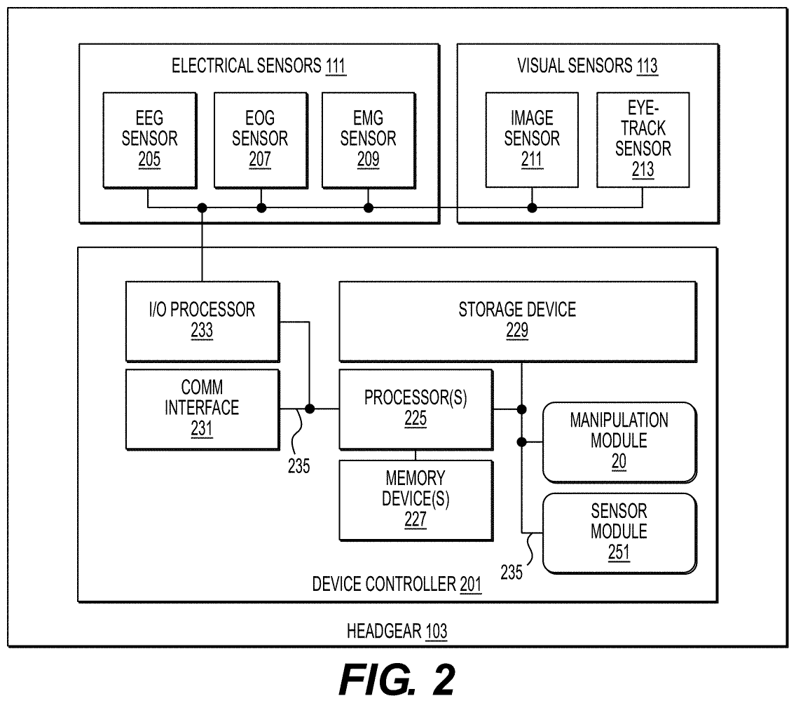

FIG. 2 is a system block diagram illustrating an example of an environment for implementing systems and processes disclosed herein. Electrical sensors 111 can include any applicable sensors such as, for example, an EEG sensor 205, an EOG sensor 207, and an EMG sensor 209 that generate EEG data, EOG data, and EMG data which can be the same or similar to that previously described. Although EEG sensor 205, EOG sensor 207, and EMG sensor 209 are illustrated as separate sensors, it is understood that one or more of the EEG sensors, the EOG sensor, and the EMG sensor can be combined.

Sensory information sensors 113 can include an image sensor 211 and an eye-tracking sensor 213 that generate facial recognition data and eye-tracking data, which can be the same or similar to those previously described. Although image sensor 211 and the eye-track sensor 213 are illustrated as separate sensors, it is understood that image sensor 211 and eye-track sensor 213 can be combined.

Device controller 201 may be or may be part of device controller 115 of FIG. 1B and may be one or more devices that process data generated by the EEG sensor 205, the EOG sensor 207, EMG sensor 209, the image sensor 211, and the eye-track sensor 213. In some implementations, the device controller 201 can include a processor 225, a memory device 227, a storage device 229, a communication interface 231, an input/output (I/O) processor 233, and data buses 235. Device controller 201 may include signal manipulation module 20 or signal manipulation module 20 may be independent of device controller 201 and/or signal capture device 10 in general. For example, signal manipulation module 20 may be part of an analysis component remote or distinct from signal capture device 10.

In some implementations, processor(s) 225 can include one or more microprocessors, microchips, or application-specific integrated circuits. Memory device 227 can include one or more types of random-access memory (RAM), read-only memory (ROM), and cache memory employed during execution of program instructions. Processor 225 can use data buses 235 to communicate with memory device 227, storage device 229, communication interface 231, an image processor, and/or spatial sensors. Storage device 229 can include a computer-readable, non-volatile hardware storage device that stores information and program instructions.

For example, the storage device 229 can be one or more of flash drives and/or hard disk drives. A transmitter/receiver may be used to communicate signals and can be one or more devices that encodes/decodes data into wireless signals, such as a ranging signal.

Processor 225 may execute program instructions (e.g., an operating system and/or application programs), which can be stored in the memory device 227 and/or the storage device 229. Processor can also execute program instructions of a sensor module 251. The sensor module 251 can include program instructions that process the data generated by the EEG sensor 205, the EOG sensor 207, the EMG sensor 209, the image sensor 211, and the eye-track sensor 213. Processing can include filtering, amplifying, and normalizing the data to, for example, remove noise and other artifacts. It is noted that the device controller 201 is only representative of various possible equivalent-computing devices that can perform the processes and functions described herein. To this extent, in some implementations, the functionality provided by the device controller 201 can be any combination of general and/or specific purpose hardware and/or program instructions. In each implementation, the program instructions and hardware can be created using standard programming and engineering techniques.

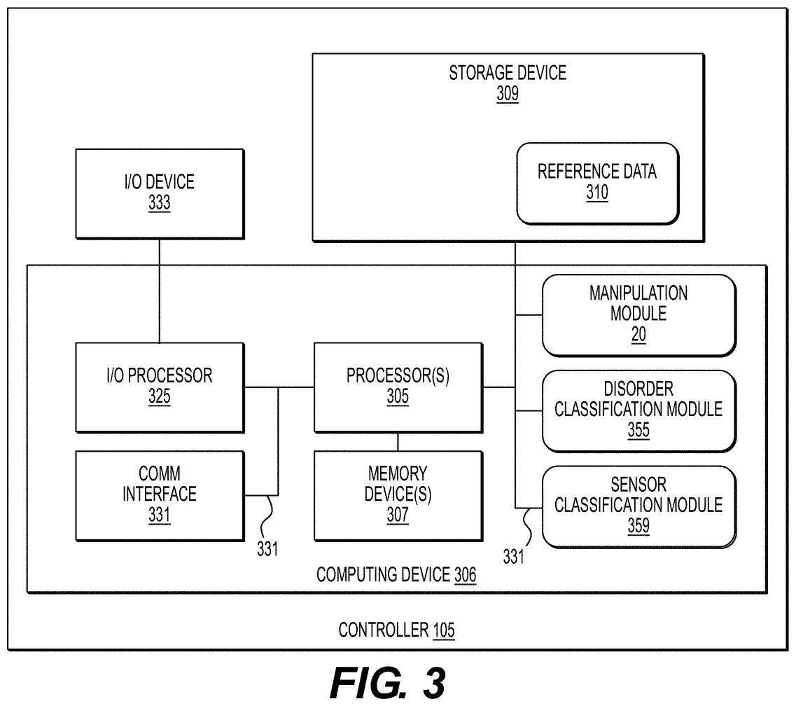

FIG. 3 shows a functional block diagram illustrating a controller 105 in accordance with aspects of the present disclosure. Controller 105 can be the same or similar to that previously described herein. Controller 105 can include a computing device 306 with a processor 305, a memory device 307, a storage device 309, and I/O processor 325, and a data bus 331. Also, controller 105 may include input connections (e.g., image input connections), and/or output connection (e.g., image output connections) that receive and/or transmit image signals from an image processor. Further, controller 105 can include input/output connections that receive/transmit data signals from I/O processor 325.

In implementations, controller 105 can include one or more microprocessors, microchips, or application-specific integrated circuits. The memory device 307 can include one or more types of random-access memory (RAM), read-only memory (ROM) and cache memory employed during execution of program instructions. Additionally, the controller 105 can include one or more data buses 331 by which it communicates with the memory device 307, the storage device 309, and the I/O processor 325.

The storage device 309 can include a computer-readable, non-volatile hardware storage device that stores information and program instructions. For example, the storage device 309 can be one or more, flash drives and/or hard disk drives. Storage device 309 may include reference data 310 for access via communication data bus 331.

I/O processor 325 can be connected to the processor 305. I/O processor 325 can include any device that enables an individual to interact with the processor 305 (e.g., a user interface) and/or any device that enables the processor 305 to communicate with one or more other computing devices using any type of communications link. I/O processor 325 can generate and receive, for example, digital and analog inputs/outputs (e.g., electronic signals) according to various data transmission protocols.

Processor 305 executes program instructions (e.g., an operating system and/or application programs), which can be stored in the memory device 307 and/or the storage device 309. The processor 305 can also execute program instructions of module 351.

Controller 105 may include signal manipulation module 20 or signal manipulation module 20 may be independent of controller 105. For example, signal manipulation module 20 may be part of an analysis component remote or distinct from signal capture device 10. Controller 105 may include a disorder classification module 355 and/or a sensor classification module 359. Disorder classification module 355 and/or a sensor classification module 359 may apply statistical analysis techniques disclosed herein to identify and/or apply clinically relevant features for disorder and/or sensor signal classification.

Controller 105 may include a communication interface 311 that facilitates inter controller or intra controller communications (e.g., via data bus 331). Controller 105 may also include one or more I/O devices 333 for communication with I/O processor 325. According to an implementation, I/O devices may include device controller 201 and/or device controller 115.

It is noted that the controller 105 is only representative of various possible equivalent-computing devices that can perform the processes and functions described herein. To this extent, in some implementations, the functionality provided by the controller 105 can be any combination of general and/or specific purpose hardware and/or program instructions. In each implementation, the program instructions and hardware can be created using standard programming and engineering techniques.

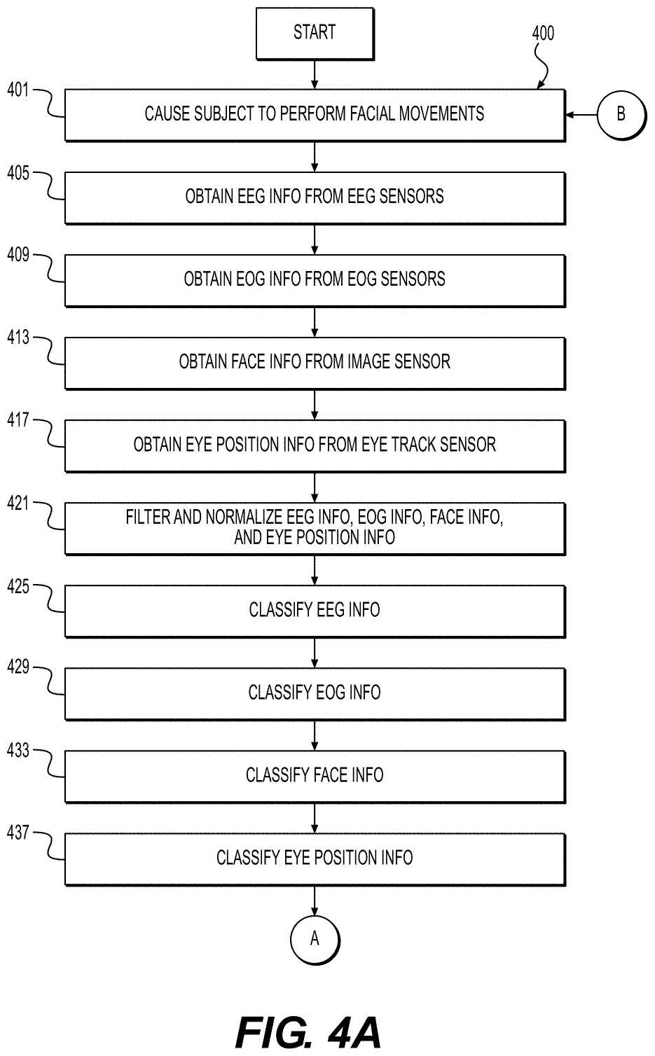

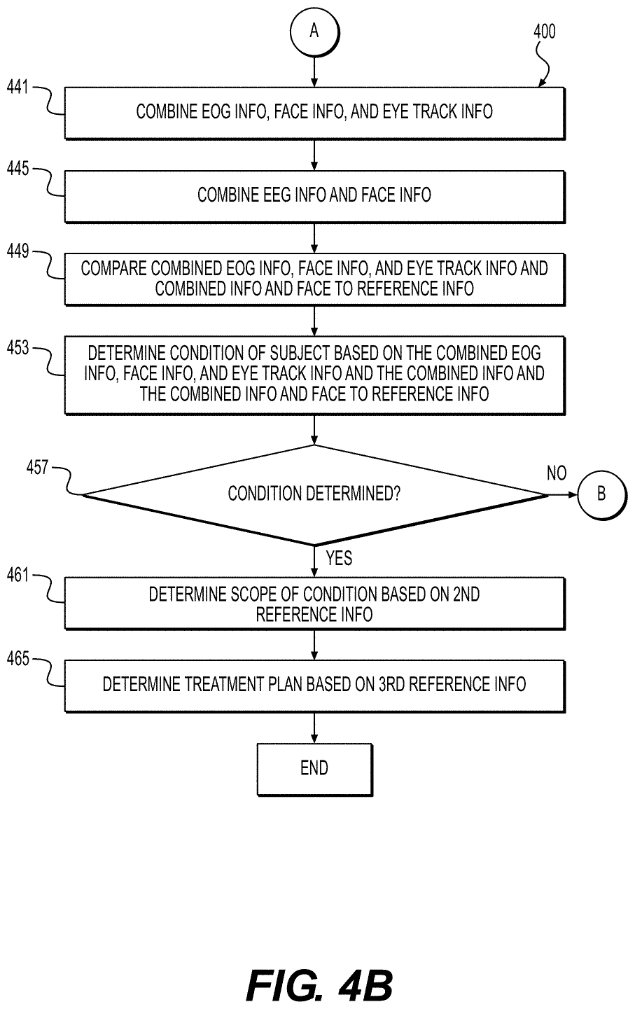

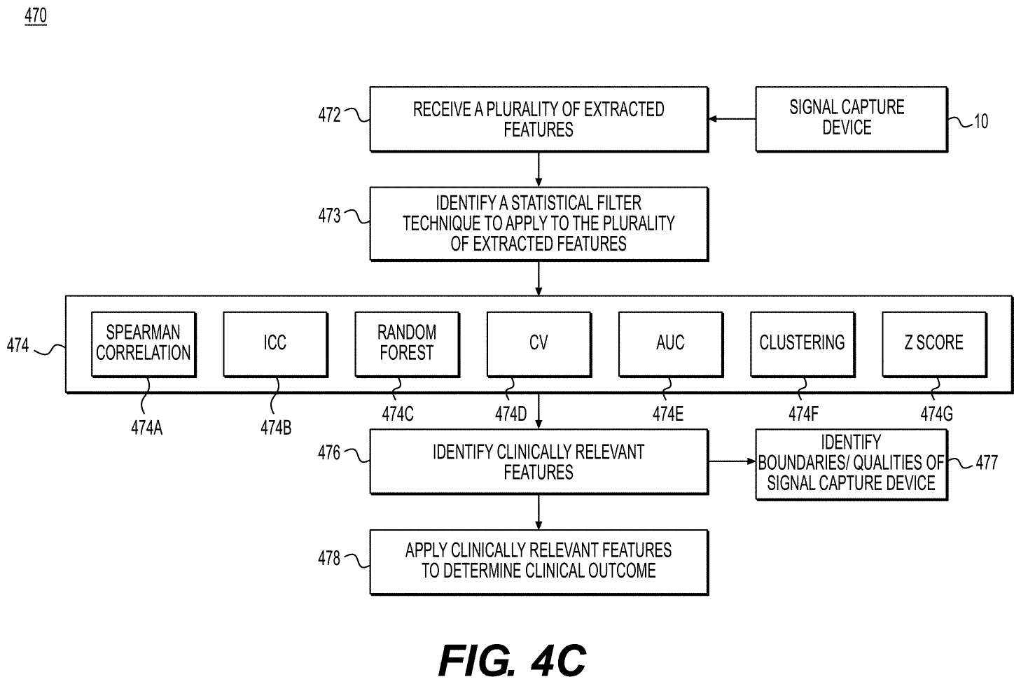

FIGS. 4A and 4B include flow diagram 400 illustrating an example of an implementation disclosed herein. As discussed herein, implementations of the disclosed subject matter may be used to identify clinically relevant features for a given clinical output, based on the extracted features from signal data. FIGS. 4A and 4B describe an example related to using a wearable device to generate signal data based on sensors on a wearable device. FIGS. 4A and 4B illustrate an example of the functionality and operation of possible implementations of systems, methods, and computer program products according to various implementations consistent with the present disclosure.

Each block in the flow diagram of FIG. 4A or 4B can represent a module, segment, or portion of program instructions, which includes one or more computer executable instructions for implementing the illustrated functions and operations. In some alternative implementations, the functions and/or operations illustrated in a particular block of the flow diagram can occur out of the order shown in FIG. 4A or 4B.

For example, two blocks shown in succession can be executed substantially concurrently, or the blocks can sometimes be executed in the reverse order, depending upon the functionality involved. It will also be noted that each block of the flow diagram and combinations of blocks in the block can be implemented by special purpose hardware-based systems that perform the specified functions or acts, or combinations of special purpose hardware and computer instructions.

At 401 of flow diagram 400, a subject may perform facial movements. The facial movements may be performed based on a request, based on a natural state of the subject, or the like. It will be understood that although facial movements are specifically disclosed herein, any body component, action, or property may be observed as part of the disclosed subject matter. At 405, EEG information may be obtained from one or more EEG sensors on a wearable device. The EEG information may be collected based on contact or contact free reception of signals via electrodes. At 409, EOG information may be obtained from one or more EOG sensors on a wearable device. The EOG information may be collected based on contact or contact free reception of signals via electrodes.

At 413, face information may be obtained from a sensory information sensor such as an image sensor. The face information may include movement, attribute information (e.g., temperature, length, elasticity, angles, etc.), or the like. The face information may be captured using a sensory information sensor based on a trigger (e.g., a request for facial information, a facial action or change, etc.) or may be captured on a continuous basis. At 418, eye-position information may be obtained from an eye tracker sensor. The eye-position information may include movement, attribute information (e.g., degree of movement, direction of movement, dilation, angles, etc.), or the like. The eye-position information may be captured using a sensory information sensor based on a trigger (e.g., a request for eye-position information, an action or change, etc.) or may be captured on a continuous basis.

At 421, one or more of the EEG information, EOG information, face information, and/or eye position information may be filtered and/or normalized. The information may be filtered and/or normalized based on any applicable technique disclosed herein. The information may be filtered and/or normalized to remove noise, to extract properties, or the like. At 425, EEG information may be classified. At 429, EOG information may be classified. At 433, face information may be classified. At 437, eye position information may be classified.

At 441 (shown in FIG. 4B), the EOG, face, and eye tracking information may be combined. At 445, EEG information and face information may be combined. It will be understood that information may be combined based on clinical outcome. For example, 441 and 445 of FIG. 4B may be different for different clinical outcomes such that different information than the information provided in FIGS. 4A and 4B may be combined. It will be understood that any signal information related to an individual may be statistically evaluated based on the flows shown in, for example, FIGS. 1A, 4A, and 4B.

At 449, combined EOG information, face information, eye tracking information and/or other combined information may be compared to reference information. As shown in FIG. 1A, the comparison may be based on extracted features form the EOG, EEG, face information, eye position information, or any other applicable information. The comparison may be based on extracted features that are determined to be clinically relevant features, shown in FIG. 1A.

At 453, clinically relevant features based on the sensed information may be used to determine the condition of a subject. For example, the condition may be determined based on the combined EOG information, face information, and eye tracking information as well as the combined EEG and face information and the reference information. At 457, a determination may be made whether a condition has been determined. If a condition has not been determined, then steps discussed herein starting at 401 may be repeated (e.g., as indicated by “B” in FIGS. 4A and 4B). If a condition is determined at 457, then at 461, a determination of the scope of the condition based on, for example, second reference information may be determined. At 465, a treatment plan may be determined based on third reference information, and/or the second reference information (e.g. based also on the scope of the condition). FIG. 4C shows flowchart 470 for identification and application of clinically relevant features. At 472, a plurality of extracted features may be received. As discussed herein, the plurality of extracted features may be based on signals collected from or about an individual's body. The signals maybe be collected as an action is being taken or specific body part is observed (e.g., Angry impression, Chewing, Eye movement, Eye-Iso, In-Iso, Jaw, Left gaze-left, Left gaze-right, Right gaze-left, Right gaze-right, Out-Iso, Sad impression, Smile-Iso, Surprise impression, Swallowing, Talking, Up Gaze, Wrinkle-Iso, etc., as described further in Table 7). Accordingly, a given extracted feature may be based on a given type of signal (e.g., ECG, EEG, etc.), an action while the signal is collected, a time when the signal is collected, an individual, and/or the like. At 472, a plurality of available extracted features may be received.

At 473, a statistical filter technique to apply to the plurality of extracted features may be identified. The statistical filter technique may be a single technique or may be a plurality of techniques applied at once. The statistical filter technique may be identified to output clinically relevant features such that the clinically relevant features can be used to best determine a clinical outcome. As discussed herein, a clinical outcome may be identification of a disease or disorder (e.g. at 453 of FIG. 4B) and/or may be a treatment plan for an identified disease or disorder (e.g., 465 of FIG. 4B). The clinical outcome may be based on objective data (e.g., based on sensor collected signals).

At 474, the one or more statistical filter techniques identified at 473 may be applied to the plurality of extracted features received at 472. The statistical filter techniques may include, but are not limited to, spearman correlation 474A, ICC 474B, random forest algorithm 474C, CV 474D, AUC 474E, clustering 474F, Z scores 474G, and/or the like or a combination thereof. These statistical filter techniques are discussed further herein.

At 476, clinically relevant features may be identified based on the statistical filter techniques applied at 474. In identifying clinically relevant features from the plurality of features received at 472, a clinical trial or other program may be run using only the clinically relevant features based on, for example, a given clinical outcome. The identifying clinically relevant features may be features that can be used to test the clinical outcome in a reliable manner such that the identified clinically relevant features may provide reliable data for the given clinical outcome. The reliability may meet a given reliability threshold for the given clinical outcome. The given reliability threshold may be a numerical value determined based on one or more of a spearman correlation, ICC, random forest result, CV, AUC, clustering, and/or Z score associated with the data. For example, the given reliability threshold may be based on minimum or maximum values associated with one or more of the spearman correlation, ICC, random forest result, CV, AUC, clustering, and/or Z score for a given set of raw data or features. The raw data may correspond to the features received at 427. Accordingly, the reliability threshold may be a single value (e.g., a binary score, a ratio, a percentage, etc.) or a set of values (e.g., one for each of the spearman correlation, ICC, random forest result, CV, AUC, clustering, and/or Z score) that indicate that a given set of clinically relevant features provide relevant data for a given clinical outcome. At 478, the clinically relevant features may be applied to determine a clinically relevant outcome.

It will be understood that although FIG. 4C provides a set of example statistical filter techniques (spearman correlation, ICC, random forest result, CV, AUC, clustering, and/or Z score), one or more additional techniques may be applied and that the set of statistical filter techniques is not limited to those disclosed herein. Any statistical filter technique that indicates the reliability of one or more features may be used to identify clinically relevant features.

According to implementations of the disclosed subject matter, the identification of clinically relevant features at 476 of FIG. 4C may be used to determine boundaries and/or qualities of a sensor based device (e.g., signal capture device 10 of FIG. 1A) used to collect one or more signals (e.g., distinct signals 30 or signals provided to signal manipulation module 20) used to determine extracted features received at 472. By applying the techniques (e.g., a protocol) disclosed herein, at 477, clinically relevant factors for a given clinical outcome (e.g., prediction of a specific disorder and/or treatment for the specific disorder) may indicate the boundaries and/or qualities of a sensor based device. For example, clinically relevant features based on a wearable device and a given clinical outcome may be identified. Based on the clinically features (e.g., one or more frequencies, one or more type of electrical signal channels, one or more spectral entropies, one or more peak frequencies, one or more amplitudes, one or more standard deviations, one or more root mean squares, one or more deflections, one or more fluctuations, one or more fractal dimensions, one or more crossing rates, one or more amplitude kurtosis, one or more skews, one or more onset times, one or more variances, etc.), a determination may be made whether the corresponding sensor based device is reliably able to output the clinical outcome (e.g., if a threshold number of extracted features meet reliability thresholds, if the clinically relevant thresholds exceed reliability thresholds, etc.).

### EXAMPLES

Implementations of the disclosed subject matter are disclosed herein with references to examples. It will be understood that the implementations disclosed herein are not limited only to the data, orders, or specifics disclosed in the examples.

### Example 1

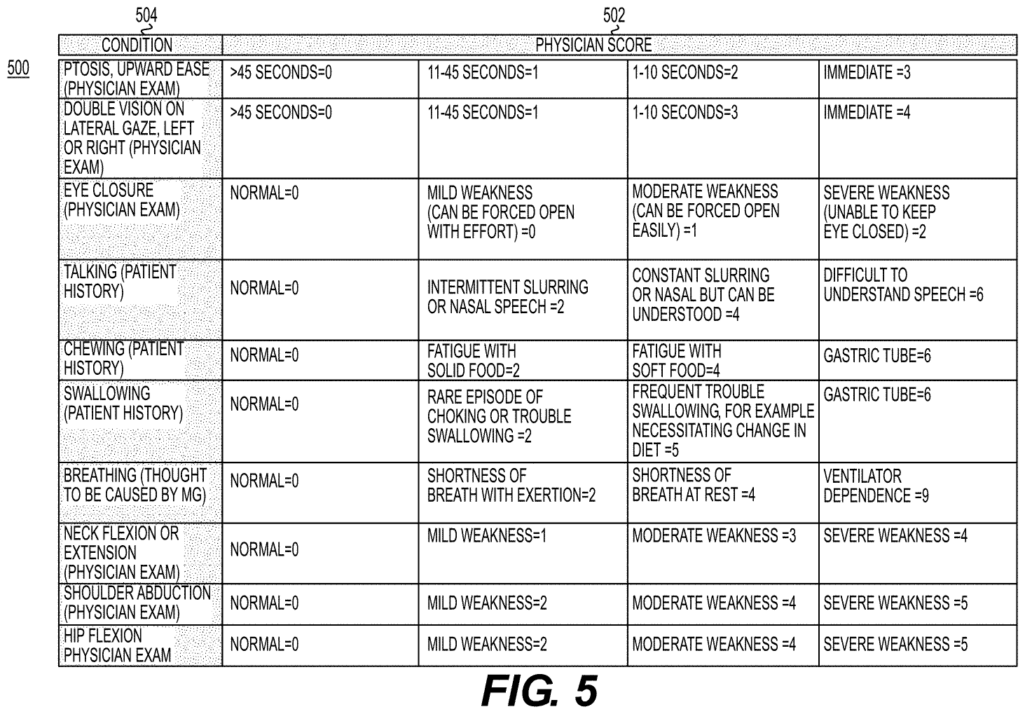

FIG. 5 includes chart 500 that shows the limitations of existing clinical outcome measures as some are subject to recall bias. For example, chart 500 may be used by a clinical professional to receive subjective physician scores 502 based on a condition 504. As an example, a healthcare provider may examine ptosis (upward ease) by observing the number of seconds a vision direction is held. A subjective physical score may be determined by the healthcare provider. However, such physical scores and corresponding assessments are subject to errors, recall biases, missed information, and can lead to misdiagnosis.

A study was conducted to perfect biometric data acquisition activities with reliability. Objective data was collected and the study was designed with the following objectives:

1) understand a biometric device's data quality and missingness (dropped values);

2) understand a biometric device's test-retest reliability; and

3) understand a biometric device's capability to quantify and distinguish between various facial muscle activities.

As disclosed herein, the objectives noted above were determined based on, for example, the statistical analysis disclosed in FIG. 4C.

A total of 16 facial and eye movement tasks were selected for the study (swallowing, chewing, talking, facial expression, eye closure, gaze at different directions). A total of N=10 controls participated in the study. During the study, data was received though the biometric device with 31 variables. Initial data exploration was conducted on measurement values.

According to implementations of the disclosed subject matter, ICC and CV analysis captures test-retest ability among variables (e.g., the 31 variables tested). Using ICC and/or CV, predictability of all variables based on task (actions) was assessed with multinomial logistic regression model. For example, a clinical focus on analyzing a “Swallowing” task (a favorable activity) was addressed.

Additional measurements from EOG sensors were received. The sensors were placed on the biometric device in proximity to an individual. A total of 60 summary variables and ICC analysis included some additional variables with high test-retest ability. Model selection was carried out (e.g., at 473 of FIG. 4C). The result of the model selection identified the best model (e.g., based on signals and/or biometric device) as a Random Forest model, for predicting activities and subjects.

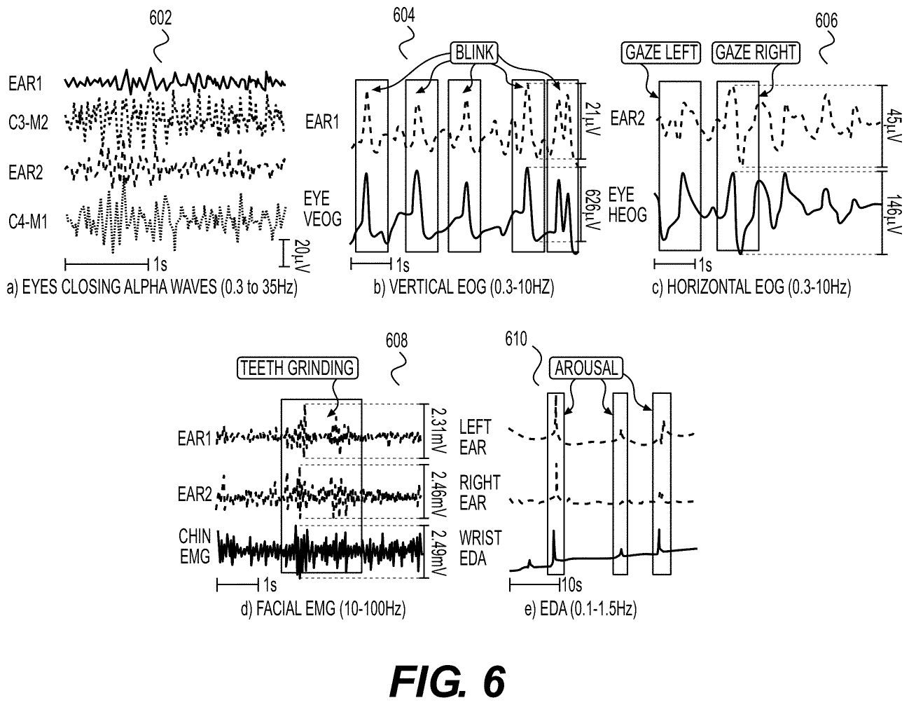

FIG. 6 shows exemplary output readings collected using one or more sensors of a biometric device. As shown in chart 602, alpha waves (0.3 to 35 Hz) were collected from various sensors while a task action of eyes closed was performed. As shown in chart 604, vertical EOG data (0.3-10 Hz) was collected from multiple sensors during a blink task. As shown in chart 606, horizontal EOG data (0.3-10 Hz) was collected during a gaze left and gaze right task. As shown in FIG. 6, a facial EMG (10-100 Hz) was collected using multiple sensors, while a teeth grinding task was performed. As shown in chart 610, an Electrodermal activity (EDA) (galvanic skin response) (0.1-1.5 Hz) was collected while an arousal task was performed.

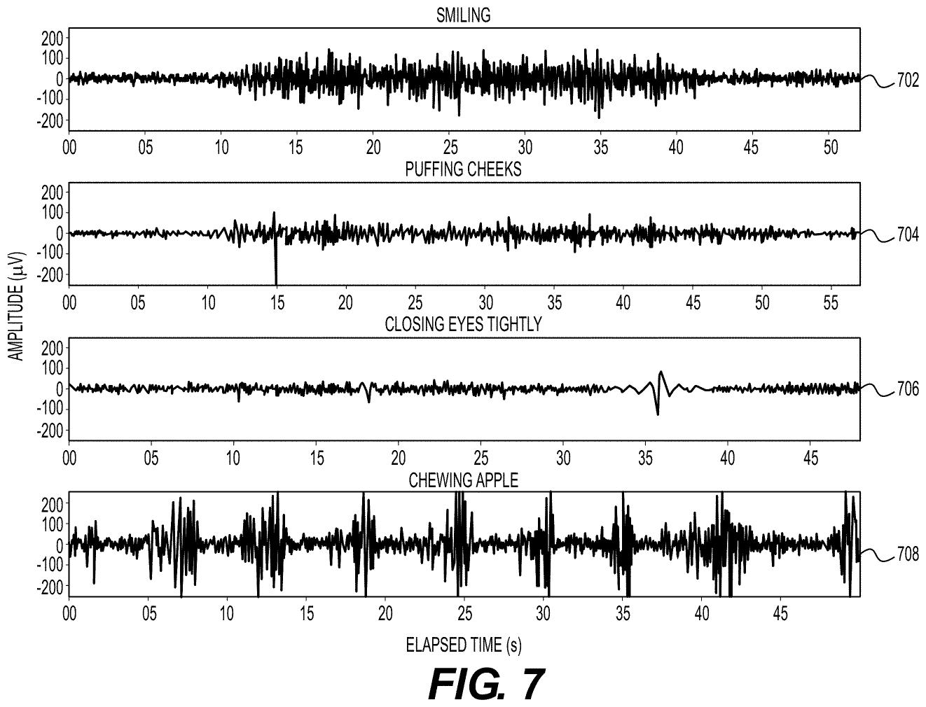

FIG. 7 shows exemplary output readings collected using one or more sensors of a biometric device. The output readings shown in FIG. 7 are collected using a device (e.g., signal capture device 10) that can reliably measure various aspects of facial biology using wave signals for brain activity, eye movement, facial muscle, or the like that was validated against polysomnography (PSG). Amplitudes (μV) collected during a smiling task are collected at 702, amplitudes collected during puffing cheeks are collected at 704, amplitudes collected while closing eyes tightly are collected at 706, and amplitudes collected while chewing an apple are collected at 708. The amplitudes shown in charts 702, 704, 706, and 708 are shown over elapsed time (seconds).

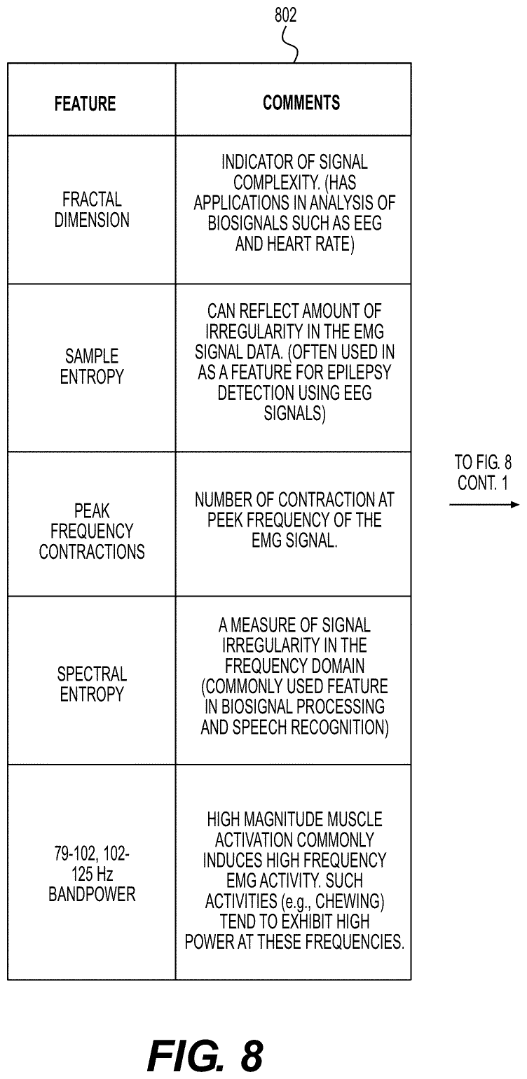

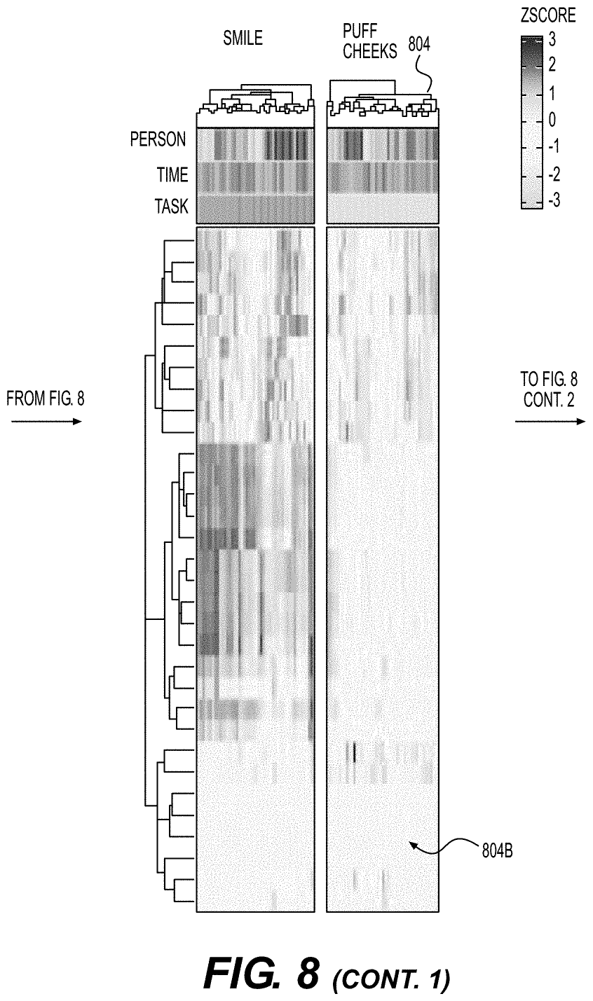

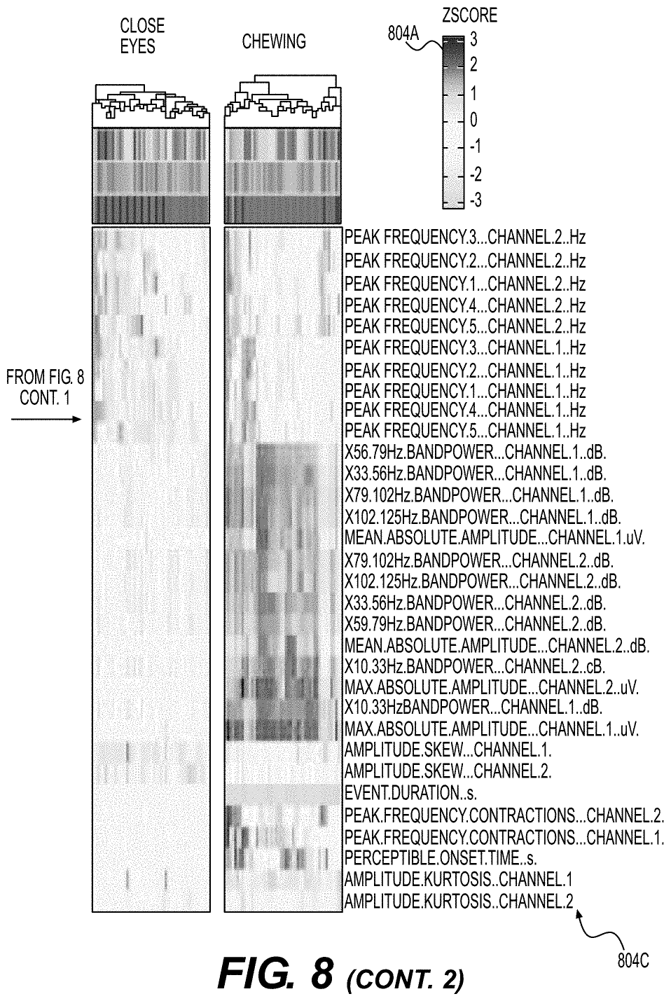

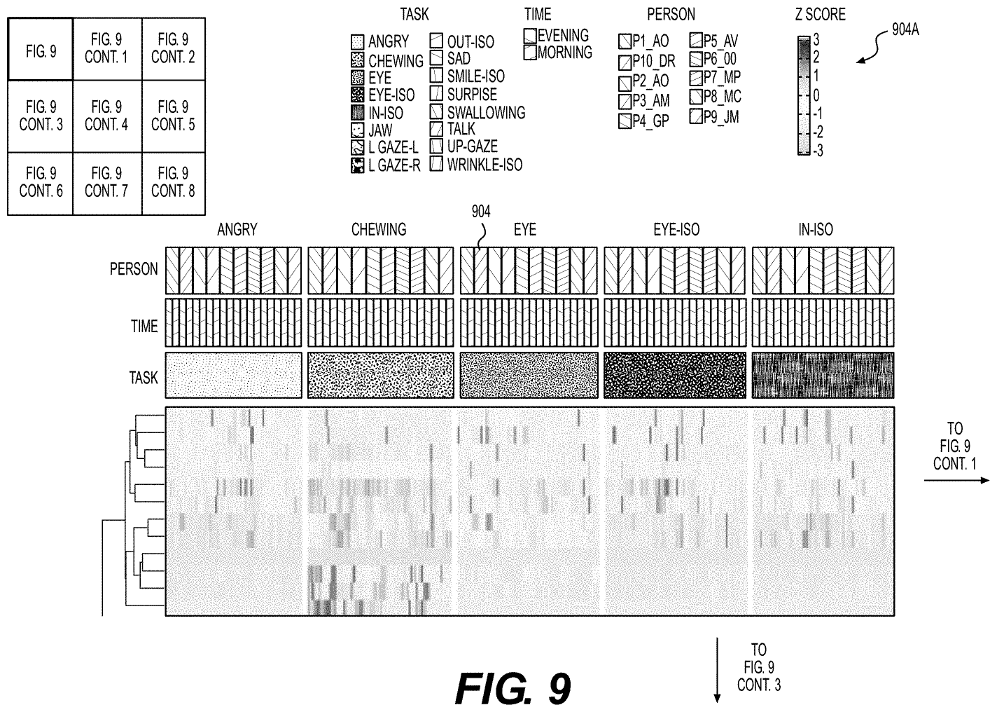

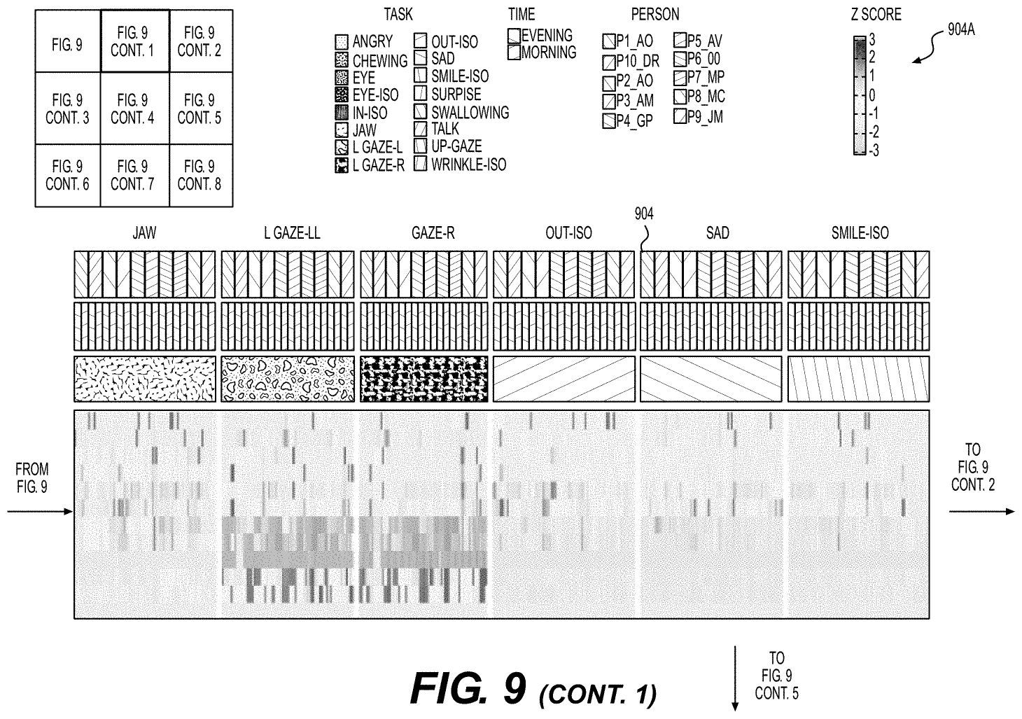

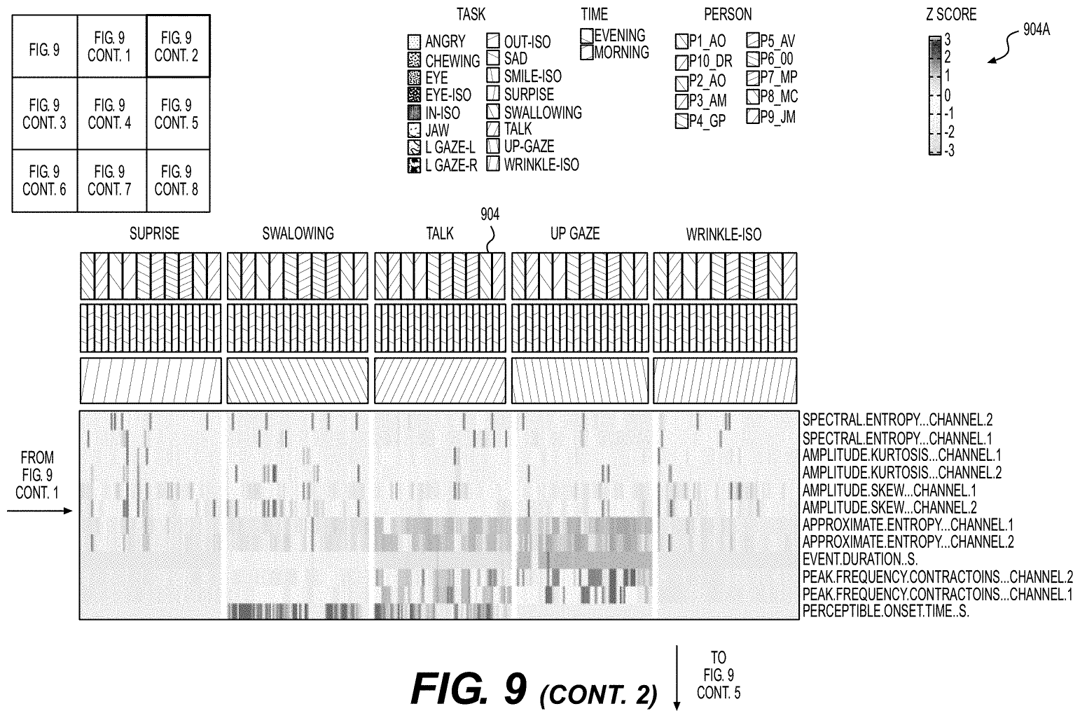

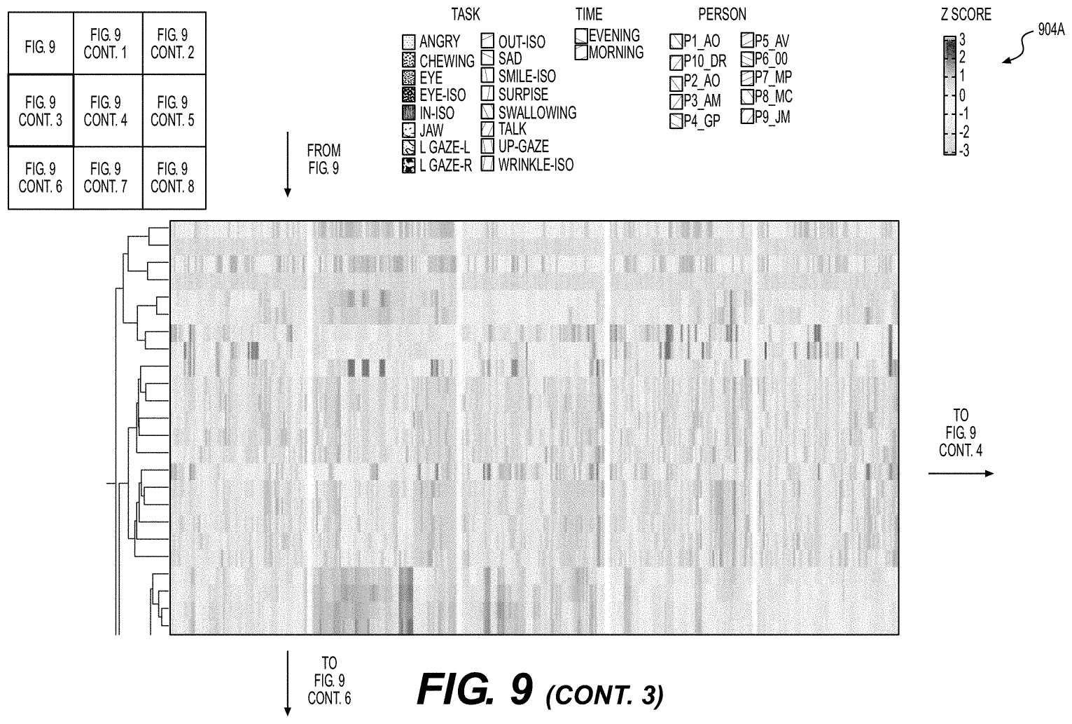

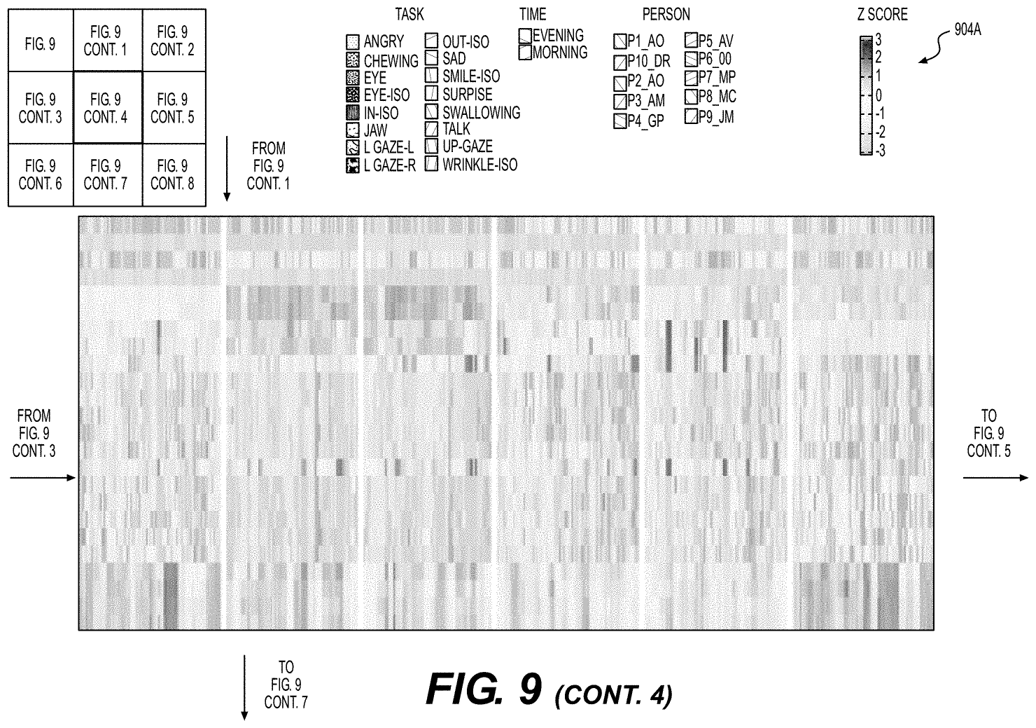

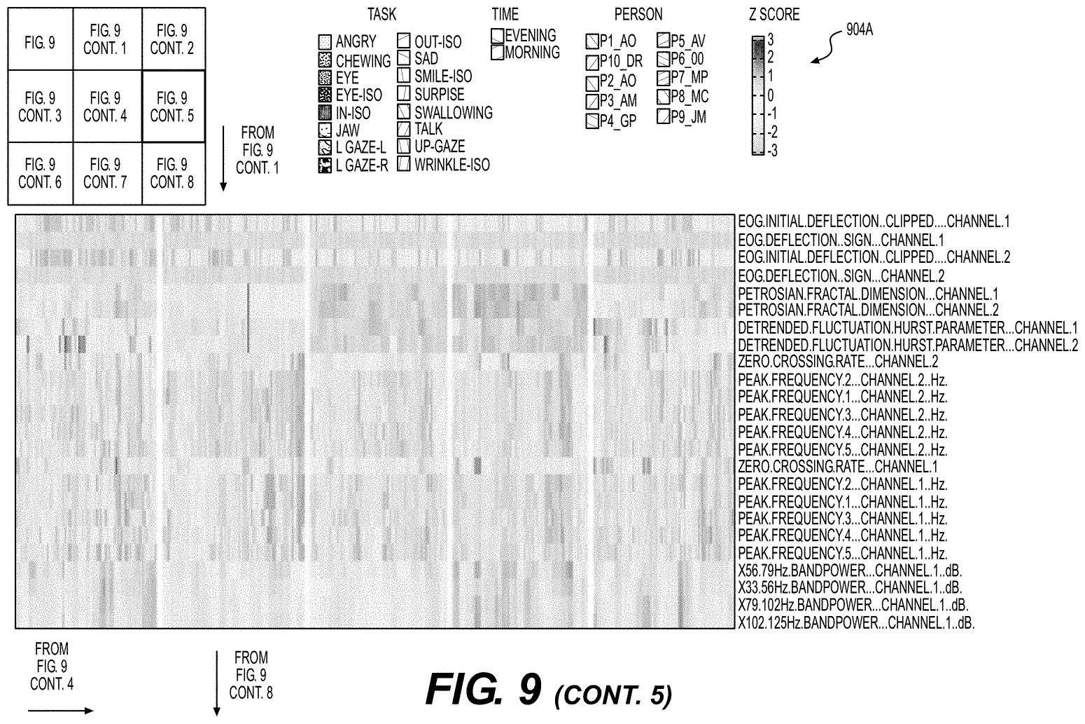

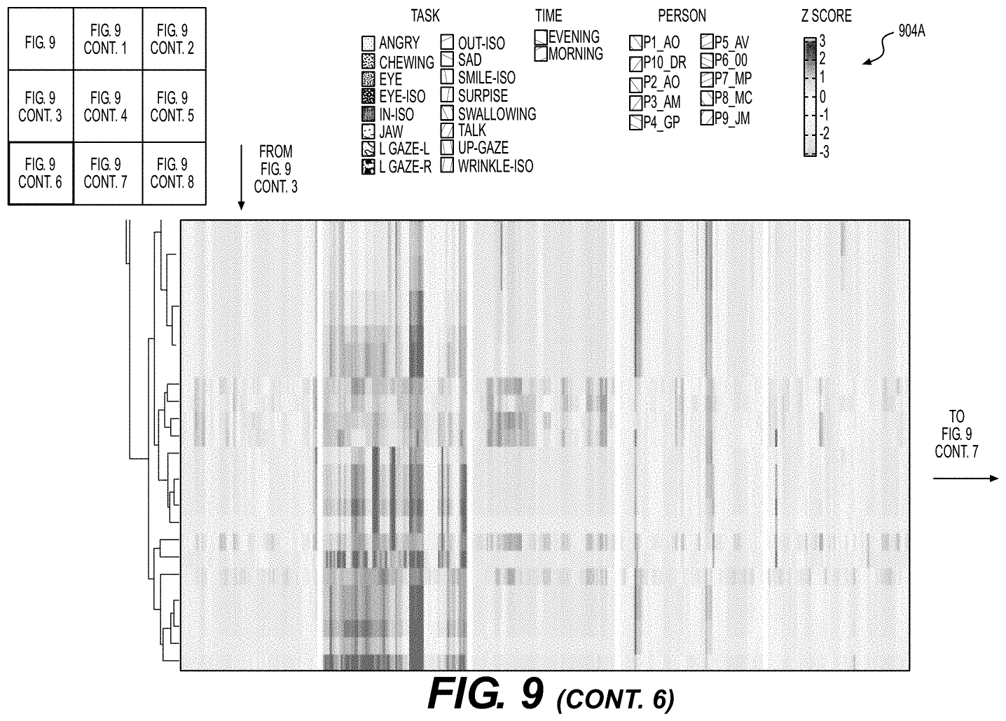

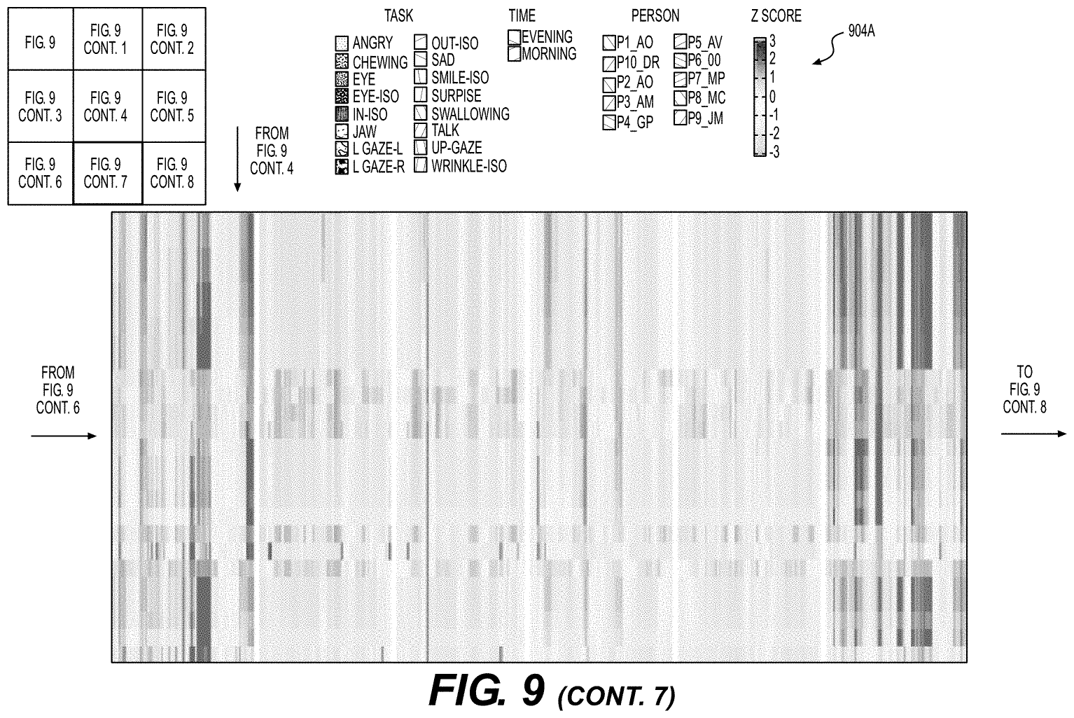

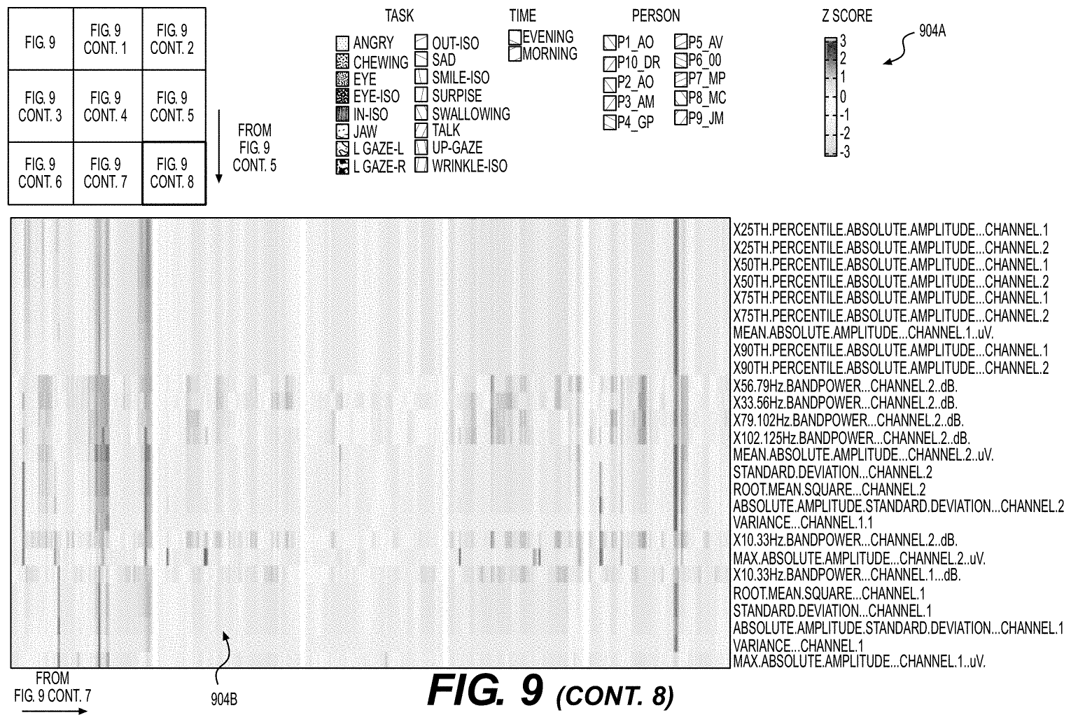

The signals shown in FIG. 6 and/or FIG. 7 may be the distinct signals 30 of FIG. 1A and/or may be provided to signal manipulation module 20 to generate distinct signals 30. Predictability based on task and individual subjects was analyzed using F1 scores, using a Random Forest model. Z scores were also obtained. The Z scores are presented herein as heat maps. Heat maps of variables and activities (tasks) demonstrates qualitative inter subject and inter activity differences, as shown in FIG. 8 and FIG. 9.

FIG. 8 is a heat map of four tasks and Z score correlation. FIG. 8 includes chart 802 that provides example feature descriptions and comments. The feature descriptions (e.g., fractal dimension, sample entropy, peak frequency contractions, spectral entropy, and bandpower) may be extracted from, for example, the signals shown in FIGS. 6 and 7 (e.g., extracted features 40). The comments associated with the features in chart 802 provide an explanation of respective figures. Chart 804 shows Z scores 804B for each of a plurality of features 804C, calculated based normalized raw data broken out by tasks (e.g., smile, puff cheeks, close eyes, chewing), individuals (persons), and time (e.g., morning/evening). The Z scores are distributed using values ranging from −3 to +3 and each value is assigned a color in accordance with legend 804A.

Similarly, FIG. 9 shows a heat map of variables and activities that demonstrates qualitative inter subject and inter activity differences. Chart 904 shows Z scores 904B for each of a plurality of features 904C, calculated based normalized raw data broken out by tasks (e.g., Angry, Chewing, Eye, Eye-Iso, In-Iso, Jaw, Left gaze-left (L Gaze L), Left gaze-right (L gaze R), Out-Iso, Sad, Smile-Iso, Surprise, Swallowing, Talk, Up Gaze, Wrinkle-Iso, as described further in Table 7), individuals (persons), and time (e.g., morning/evening). The Z scores are distributed using values ranging from −3 to +3 and each value is assigned a color in accordance with legend 904A.

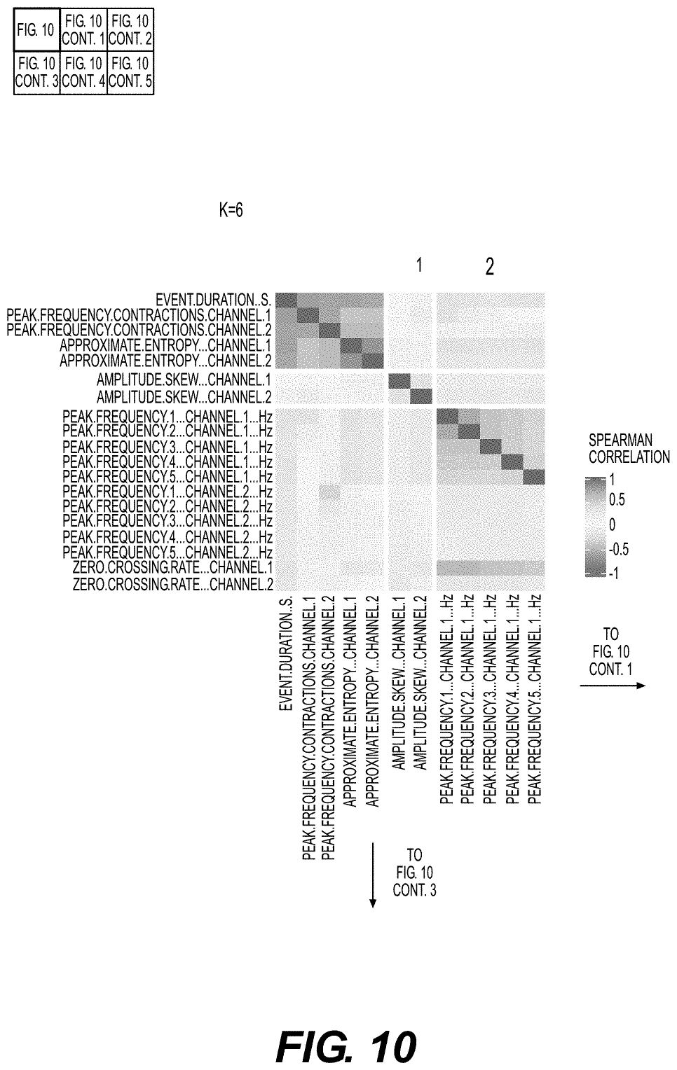

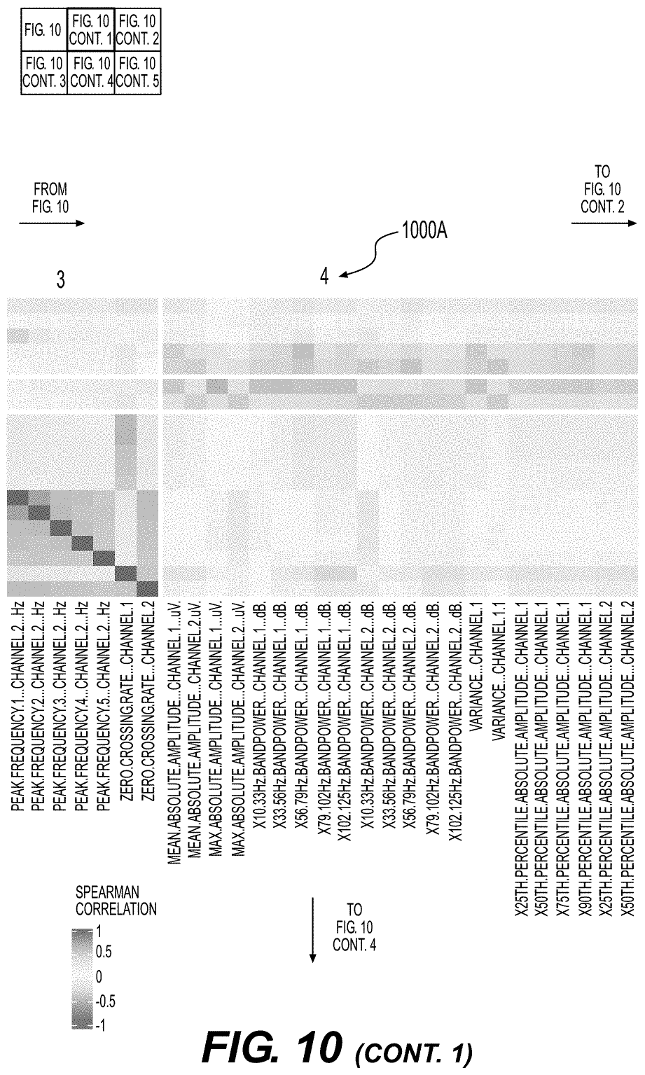

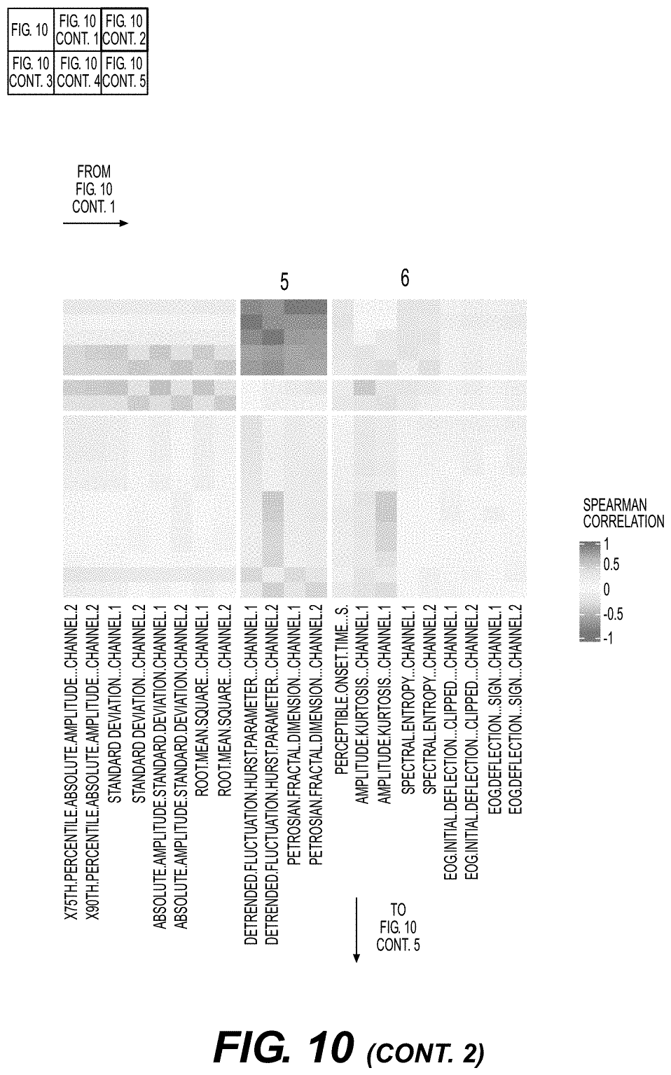

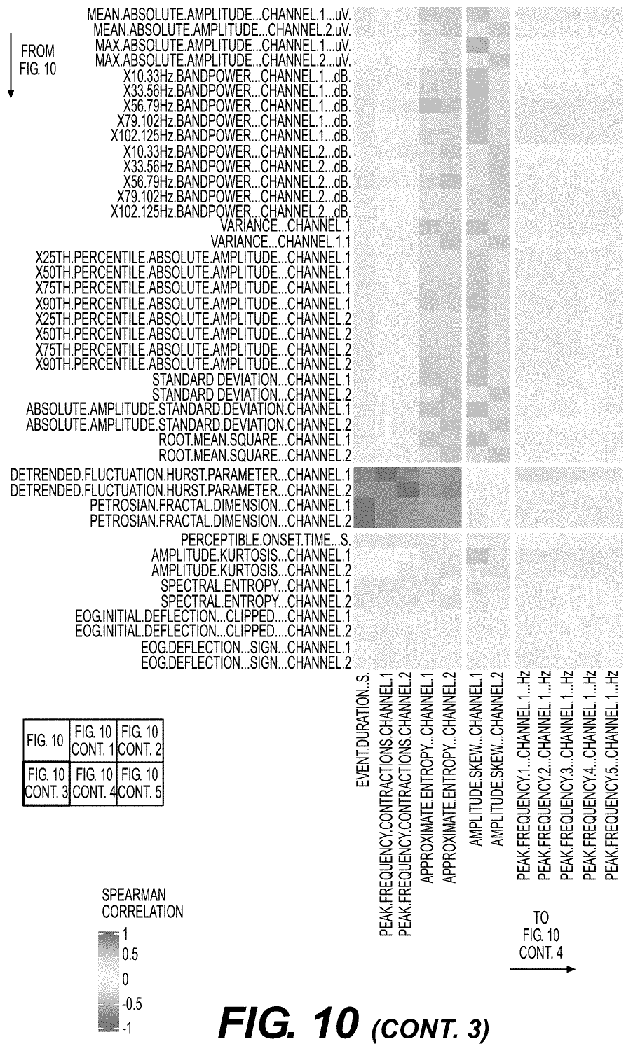

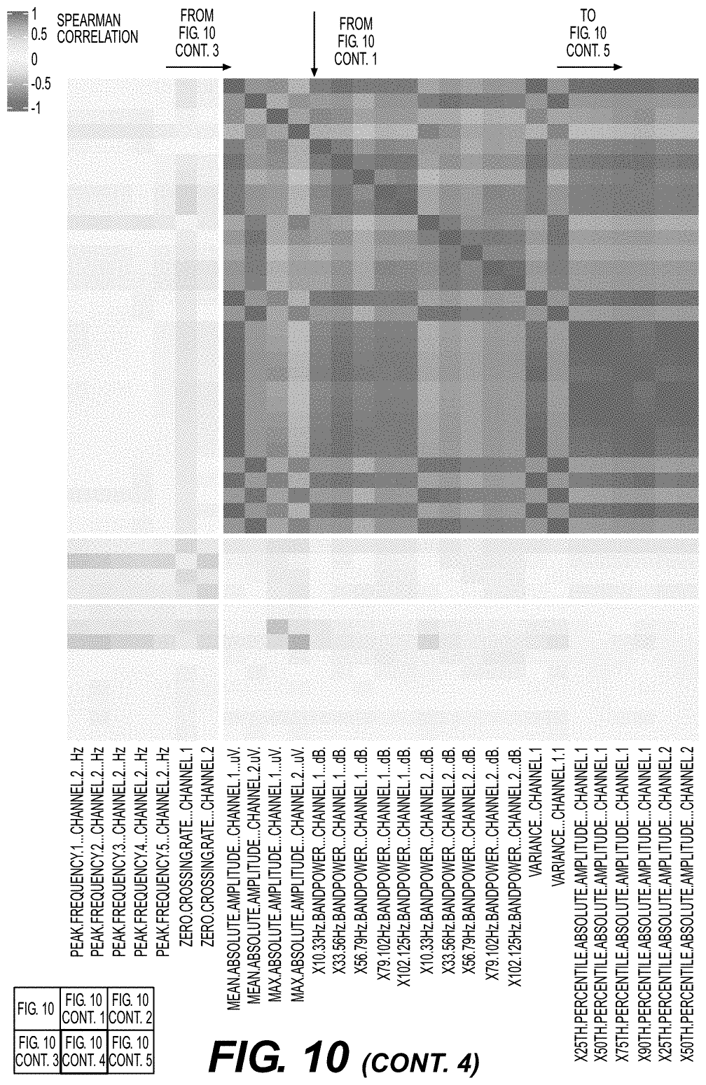

FIG. 10 shows a spearman correlation chart 1000 of each of the features 1000B that shows correlation of features 1000B against themselves. Spearmen correlation chart 1000 may be used to identify relationships between features. Features that are highly correlated are likely measuring similar aspects of facial biology and/or other signals (e.g., electrical activity) collected by signal capture device 10 (e.g., similar aspects of distinct signals 30). Spearmen correlation chart 1000 may be used to identify clusters 1000A such that similar features within the same cluster may be omitted or otherwise reduced, to reduce duplication of analysis. For example, cluster based reduction may be applied to identify clinically relevant features 50.

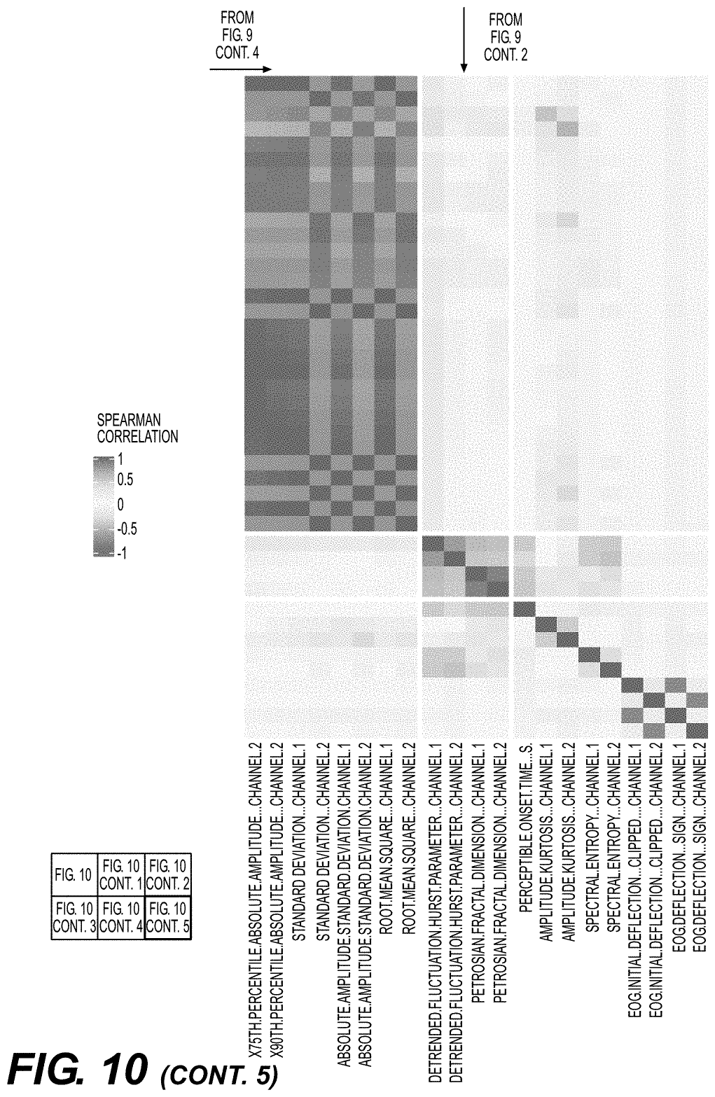

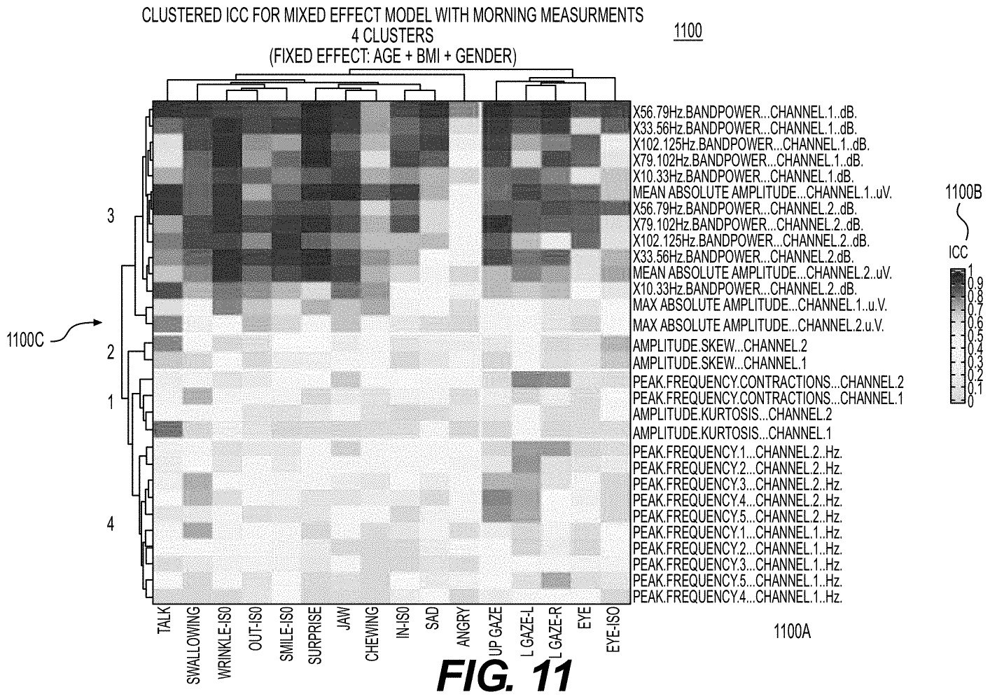

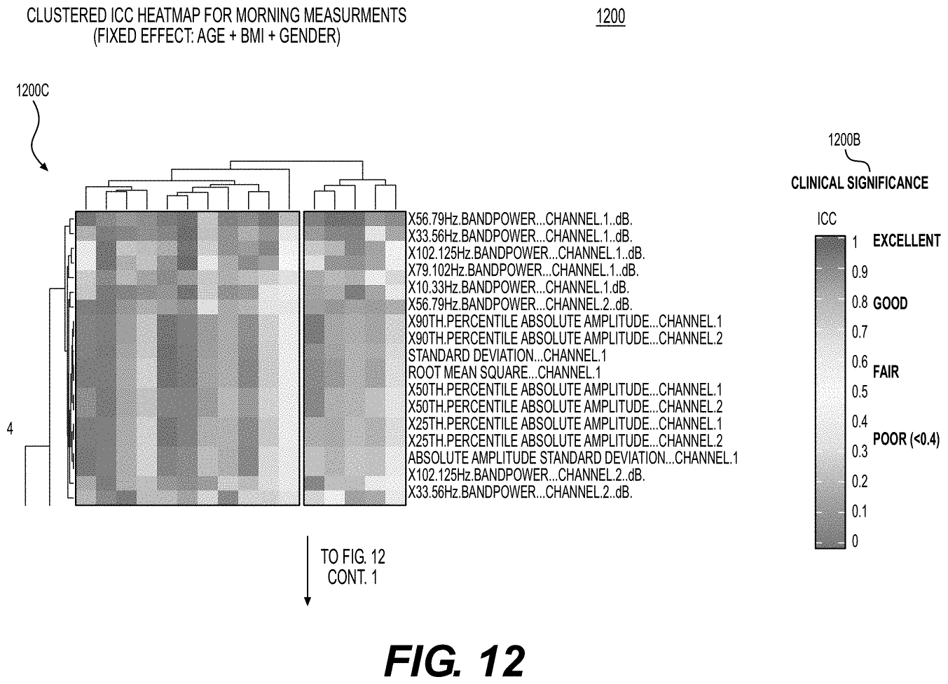

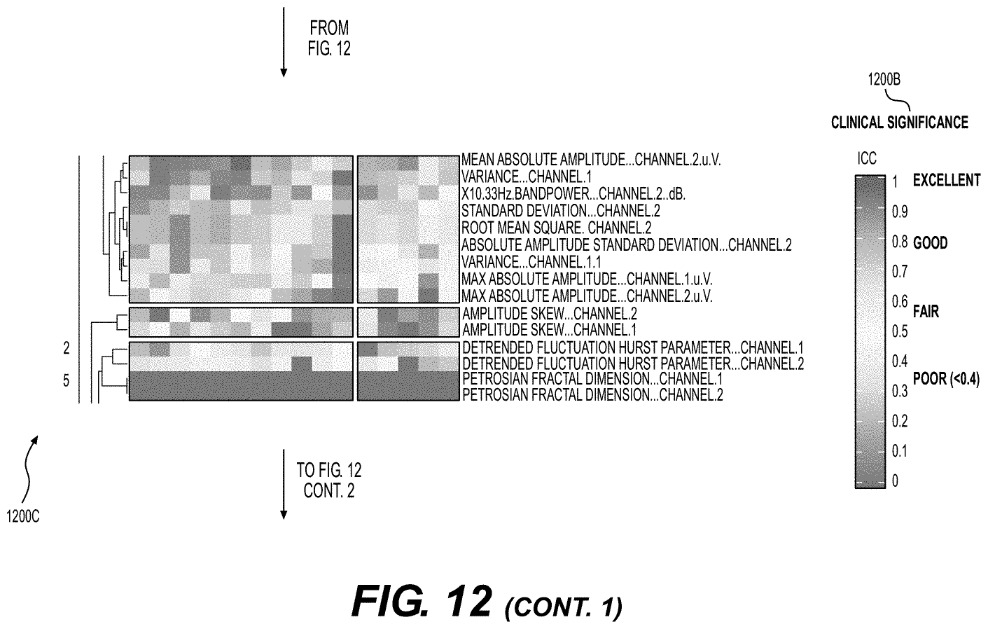

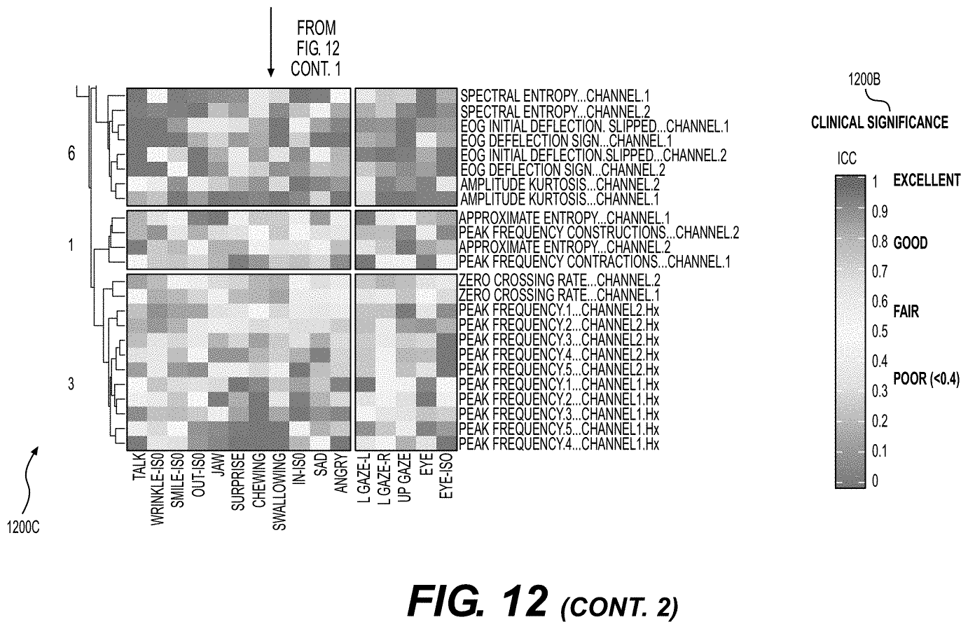

FIG. 11 shows ICC measures 1100 and FIG. 12 shows ICC measures 1200 for features 1200A that test re-test reliability of parameters and infer clinical significance. ICC measures 1100 are shown as a heat map and corresponds to morning measurement based features 1100A with fixed age, BMI, and gender. ICC measures may be measured as low clinical significance (e.g., 0) to a high clinical significance (e.g., 1) as shown in legend 1100B and 1200B, respectively.

ICC measures may be per subject (individual) such that a high clinical significance (e.g., 1) may indicate that if a given measurement is repeated for the subject, then similar data should be expected. A low clinical significance (e.g., 0) may indicate that, per subject, if a given measurement is repeated for the subject, then dissimilar data should be expected. For a given clinical outcome, a higher clinical significance (e.g., above a clinical significance threshold) may result in a given figure being designated as clinically relevant (e.g., as a clinically relevant feature 50). Higher clinical significance (e.g., in a range of 0.6 to 1) may be required for use in a clinical trial. ICC measures may be used to cluster features, as shown at 1100C and 1200C, respectively. Clusters of features with higher clinical significance may be designated as clinically relevant (e.g., as a clinically relevant feature 50).

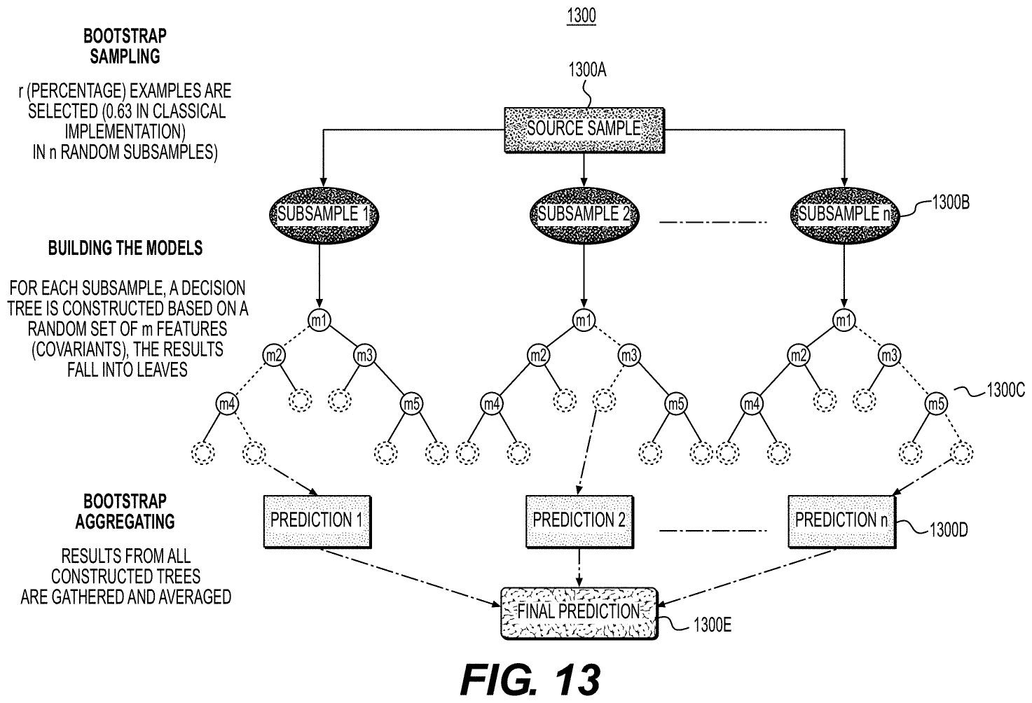

FIG. 13 shows an example prediction model 1300 (e.g., random forest model) in accordance with implementations of the disclosed subject matter. As shown in FIG. 13, bootstrap sampling may be used to build models (e.g., decision trees or networks). For bootstrap sampling, r (percentage) of examples may be selected (e.g., 0.63 in classical implementations) and may be split inn random subsamples. As shown, a source sample 1300A may be split into subsamples 1300B. For each subsample 1300B, a decision tree may be constructed at 1300C based on random set of m features (covariants), and the results may fall into leaves. At 1300D, bootstrap aggregating may be performed with results from all constructed trees that are gathered and averaged. At 1300E, a final prediction may be derived from each of the predictions at 1300D.

To optimize F1 scores, several machine learning approaches were tried, and random forests outperformed other models. Data was split: 80% training for fitting the model and 20% for testing. Random forest constructs a multitude of decision trees for predicting individual activities/subjects with training dataset and output weighted sum prediction of the individual activities/subjects.

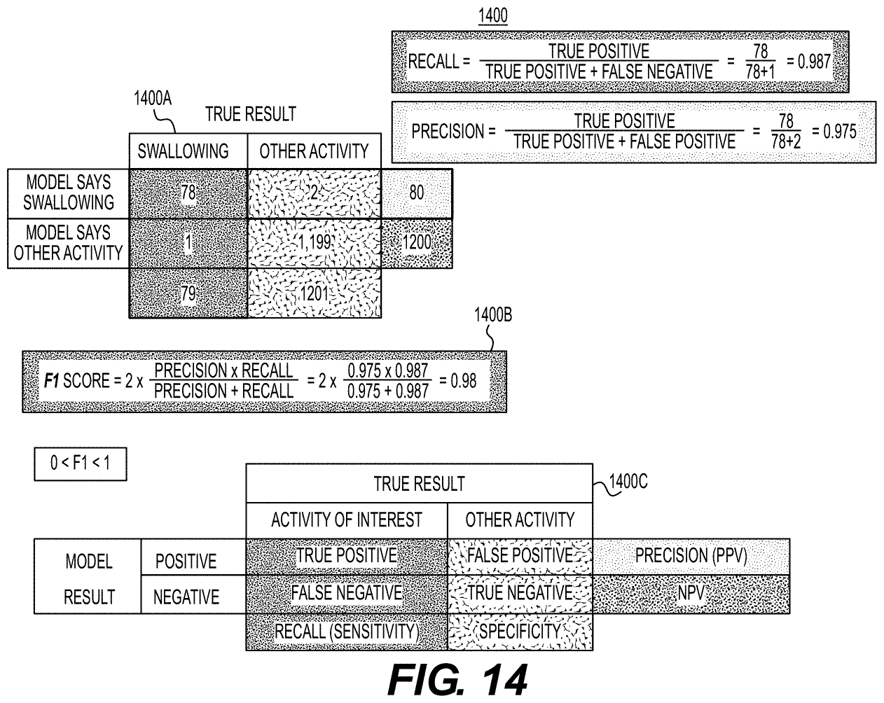

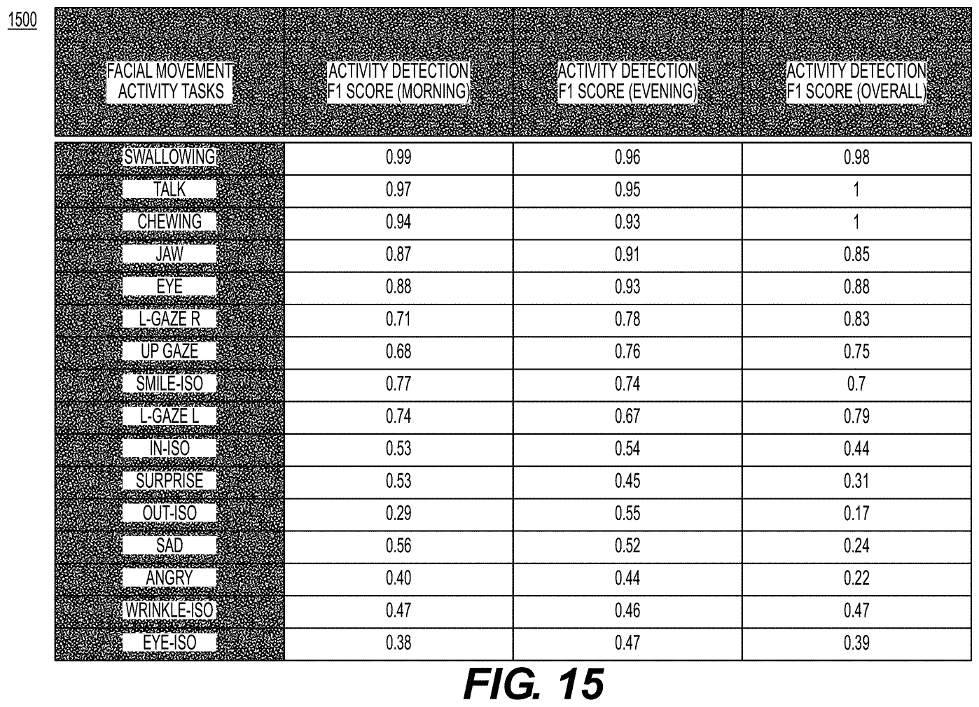

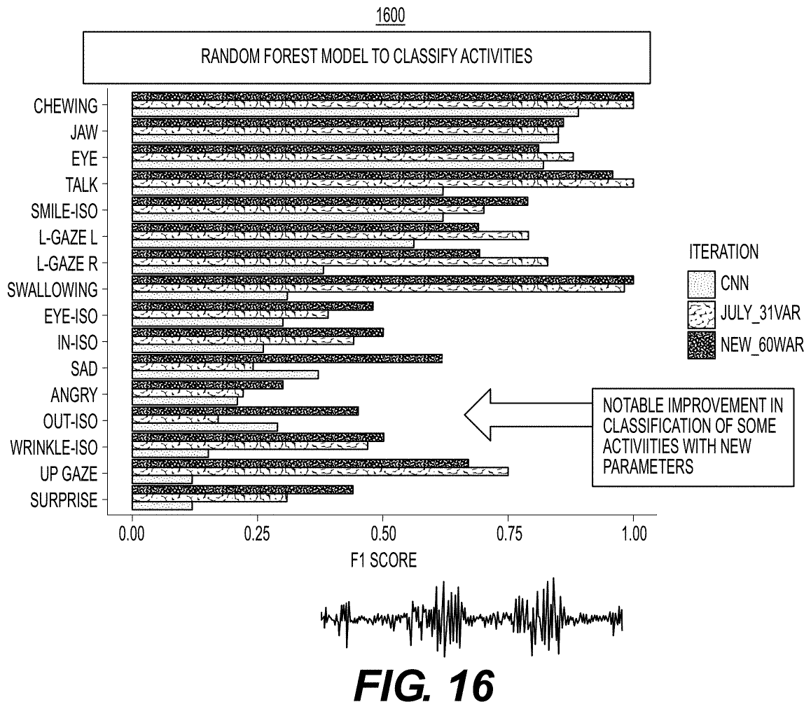

To assess the prediction accuracy from the random forest quantitatively, F1 scores were used. The F1 score measures how well a model classifies a particular activity like swallowing, as shown by the results and computations in FIG. 14 and FIG. 15. Using the parameters from both rounds of feature engineering, improvement of F1 score for some activities was observed, as indicated in FIG. 15 and FIG. 16. As shown in FIG. 14 via computation and results 1400, a recall of 0.987 and precision of 0.975 was output based on true positives, false negatives, and false positives test results 1400A. A F1 score of 0.98 was output at 1400B. 1400C shows the model used to output the criteria for the F1 score.

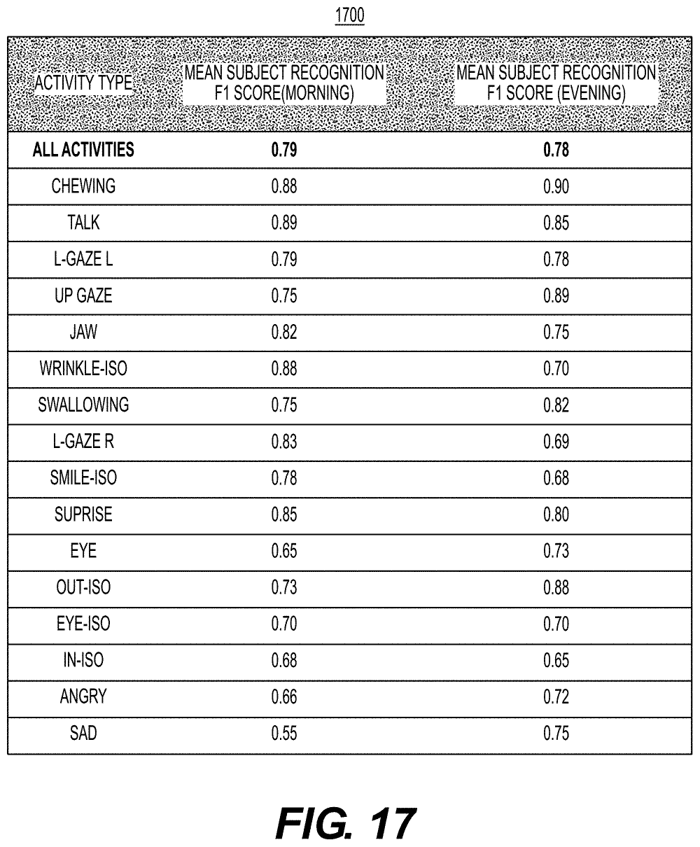

FIG. 15 shows chart 1500 F1 score results split by morning, evening, and overall. As shown, based on the signal capture device 10 used, swallowing had the highest overall F1 score at 0.98 and Eye-Iso had the lowest overall F1 score of 0.39. FIG. 16 shows graph 1600 with results from the random forest model results using a CNN model, a first variance model, and a second variance model. As shown, certain features (parameters) show improvement when compared to other features, based on the model used. FIG. 17 shows a chart 1700 with the results of the study using all variables from a particular task (activity) to predict each subject, or using all given activities to predict a subject, broken out by morning and evening scores. As shown, chewing had the highest morning and evening combined scores (0.79 and 0.90 respectively) and a sad emotion had the lowest morning and evening combined scores (0.55 and 0.75 respectively). Chewing, Wrinkle-Iso and Talk activities are among the top activities in predicting individual subject (F1 scores >0.85). Sad, eye, angry activity tracking were not as reliable in predicting individuals. Minor differences were recorded in predicting individuals from Morning to Evening. Facial movements in general measured varied across morning and evening time points in a day. Application of the device used in a clinical setting was considered with a focus on measuring chewing, talking, and swallowing, as a result of the higher F1 score based reliability.

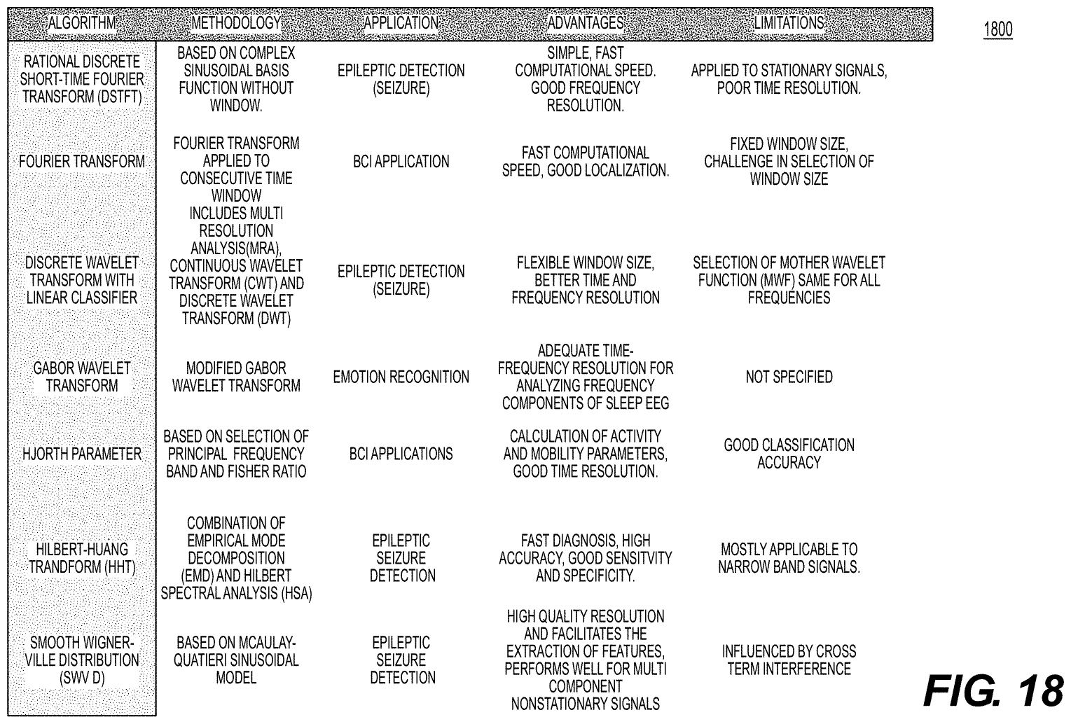

FIG. 18 shows a chart 1800 for algorithm options for selection (e.g., for application to a random forest). One or more algorithms (rational discrete short-time Fourier transform (DSTFT), Fourier transform, discrete wavelet transform with linear classifier, Gabor wavelet transform, Hjorth parameter, Hilbert-huang transform (HHT), smooth Wigner Ville distribution (SWV D)) may be applied to data collected by a signal capture device. Chart 1800 provides the methodology, application, advantages, and limitations of these example algorithms.

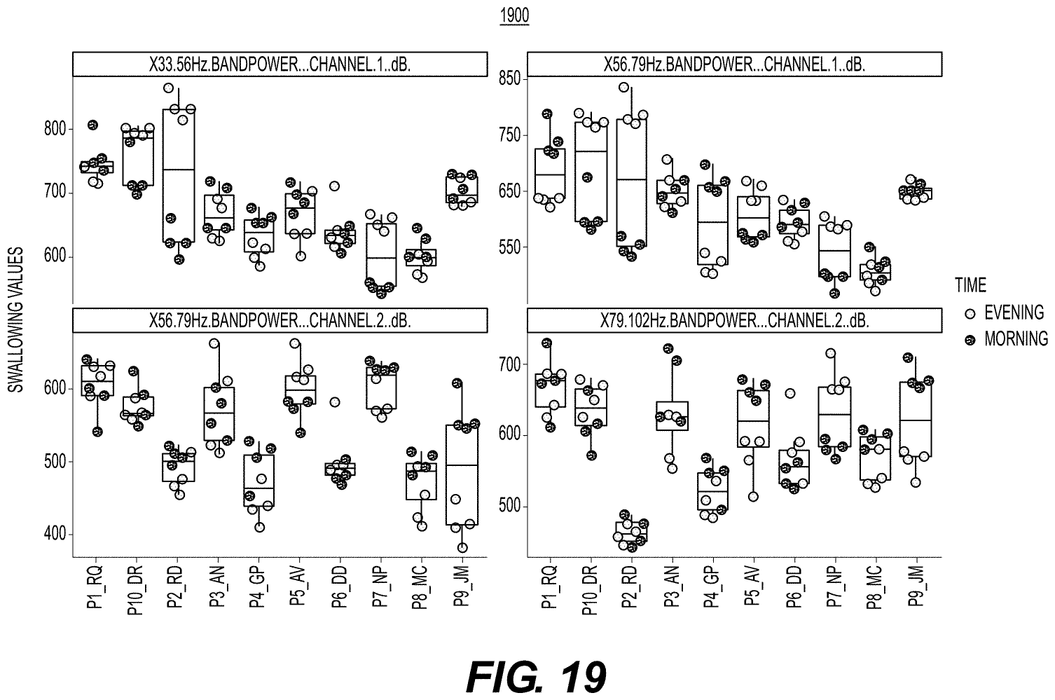

FIG. 19 shows charts 1900 of a bandpower feature for ten individuals, for a swallowing activity. Four different bandpowers are shown in the four respective charts. As shown, for each individual, multiple amplitudes are collected for each individual during both morning and evening testing times. According to an implementation, the four different bandpowers in the four respective charts may each be an endpoint in a clinical trial. For example, the data represented in the four respective charts may be the clinical output sought as the result of a clinical trial.

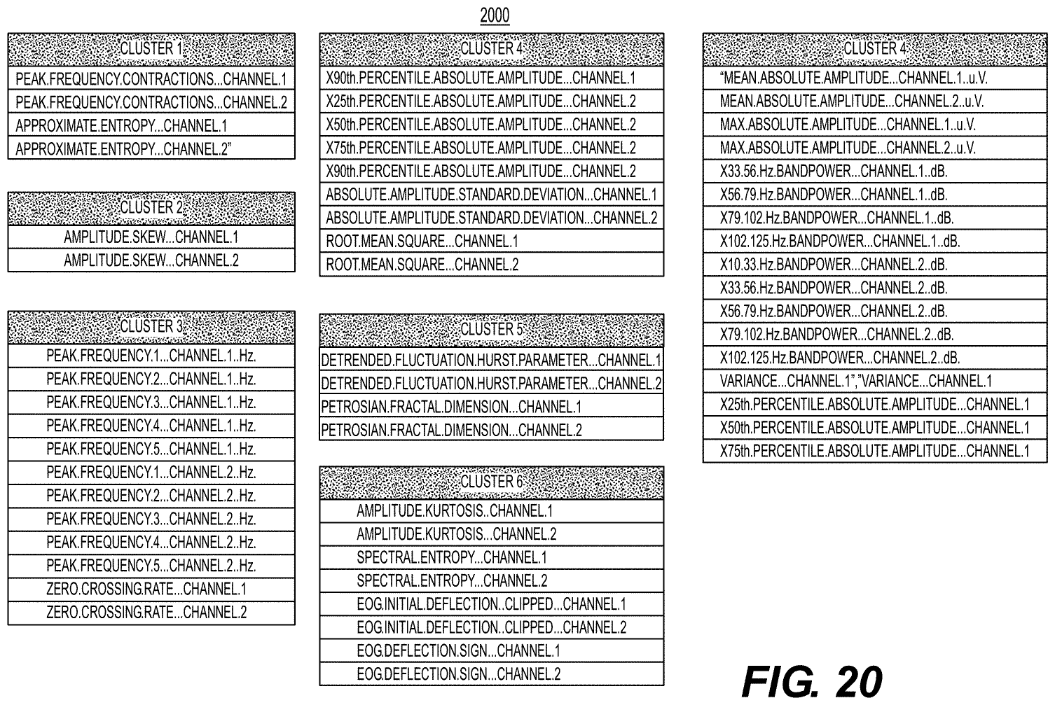

FIG. 20 shows clusters and corresponding channels in tables 2000. Each feature extracted from distinct signals 30 may be placed into a cluster based on, for example, CV clustering, ICC clustering, random forest clustering, or the like, as disclosed herein. Features with similar results may be grouped, as shown in the examples provided in tables 2000. Clusters may be used to trim a total number of features to those that may be most critically relevant to a clinical output.

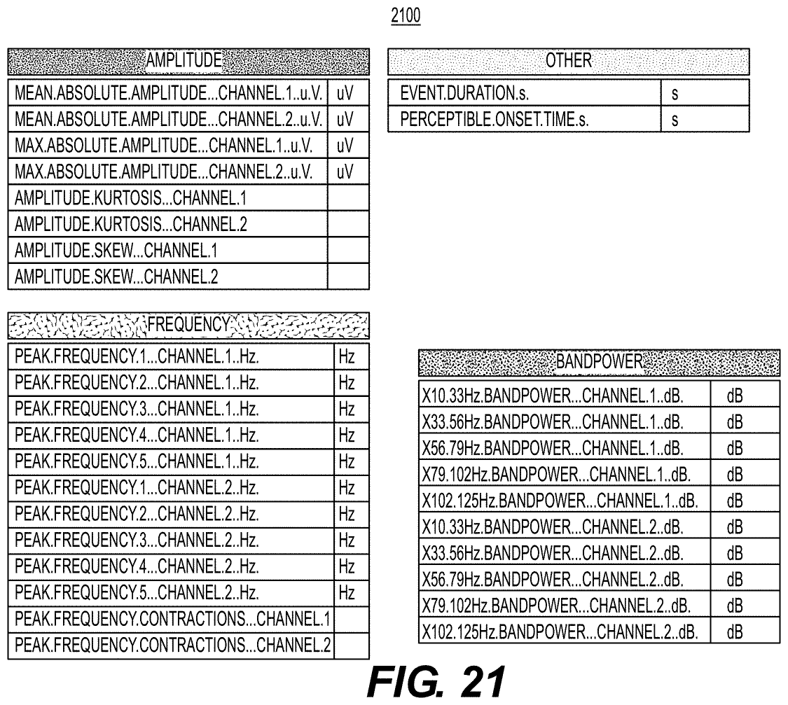

FIG. 21 shows graph 2100 with features clustered based on type (e.g., amplitude, frequency, and band-power channels, and/or other factors). Clusters may be used to trim a total number of features to those that may be most critically relevant to a clinical output.

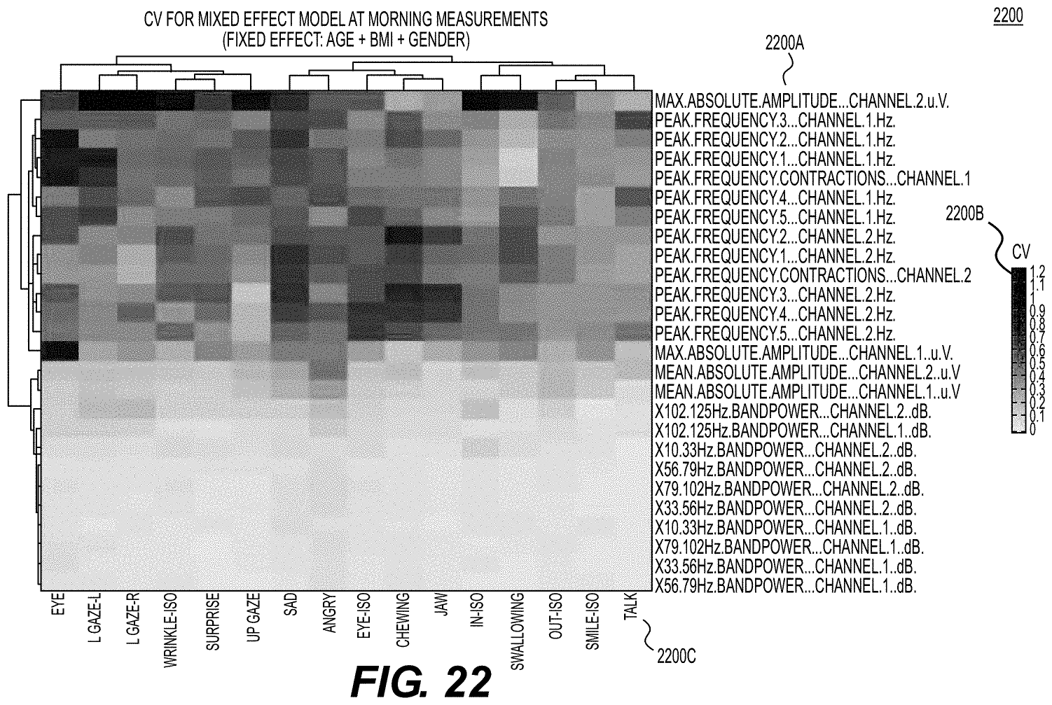

FIG. 22 shows a heat map 2200 for CVs based on a mixed effect model using morning measurements with fixed effects age, BMI, and gender. The CV heat map 2200 shows the CV values for features 2200A for tasks 2200C based on legend 2200B, which range from 0 to 1.2. The results of CV heat map 2200 may be used to identify which features will be reliable (e.g., low variance) for determining a clinical output (e.g., disease designation. For example, a lower CV for a given feature and action may indicate that the given feature can be repeated in a reliable manner (e.g., meets a CV reliability threshold) for multiple tests. A clinical trial may require that features used for the trial meet such a CV reliability threshold.

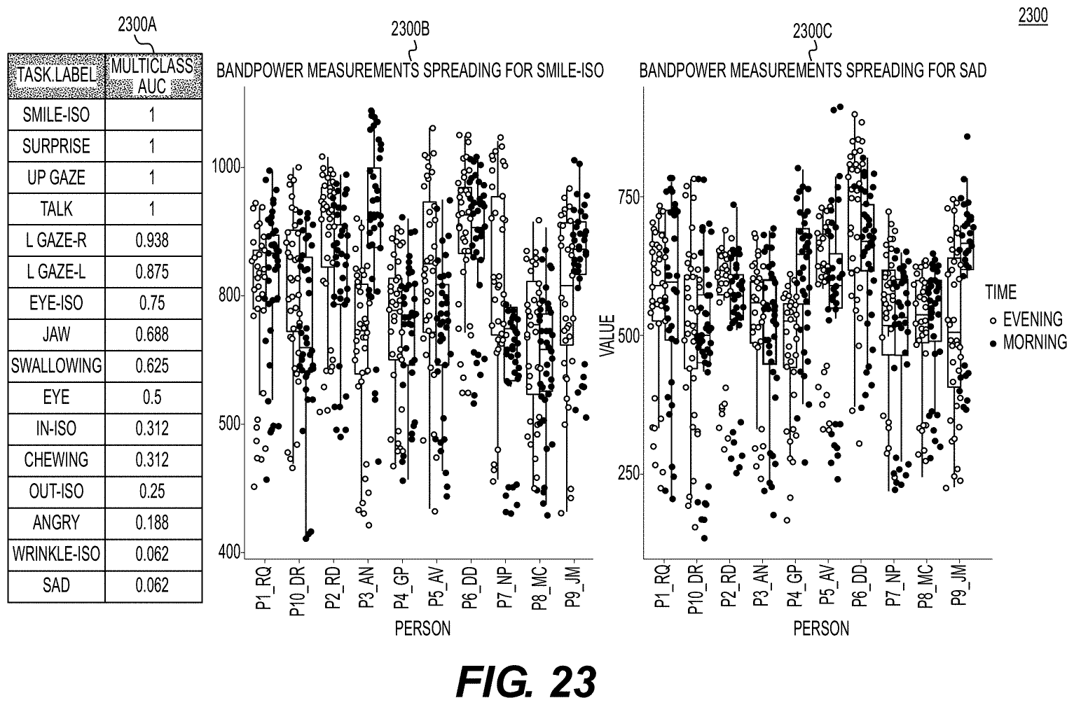

FIG. 23 shows chart 2300 that indicates how reliably a given task can be used to classify individuals. The bandpower measurements measuring AUC for various tasks are shown in table 2300A. The results of such measurements for Smile-Iso are shown in chart 2300B and for a sad emotion are shown in chart 2300C. A higher bandpower AUC measurement may indicate that a given task (e.g., Smile-Iso) meets an AUC threshold for classifying individuals (e.g., differentiating from one individual to the next), whereas a lower bandpower AUC measurement may indicate that a given task (e.g., a sad emotion) does not meet an AUC threshold. Accordingly, the signal capture device 10 used to generate the measurements shown in FIG. 23 may be more reliable in distinguishing individuals when a smile action is performed compared to when a sad emotion is experienced.

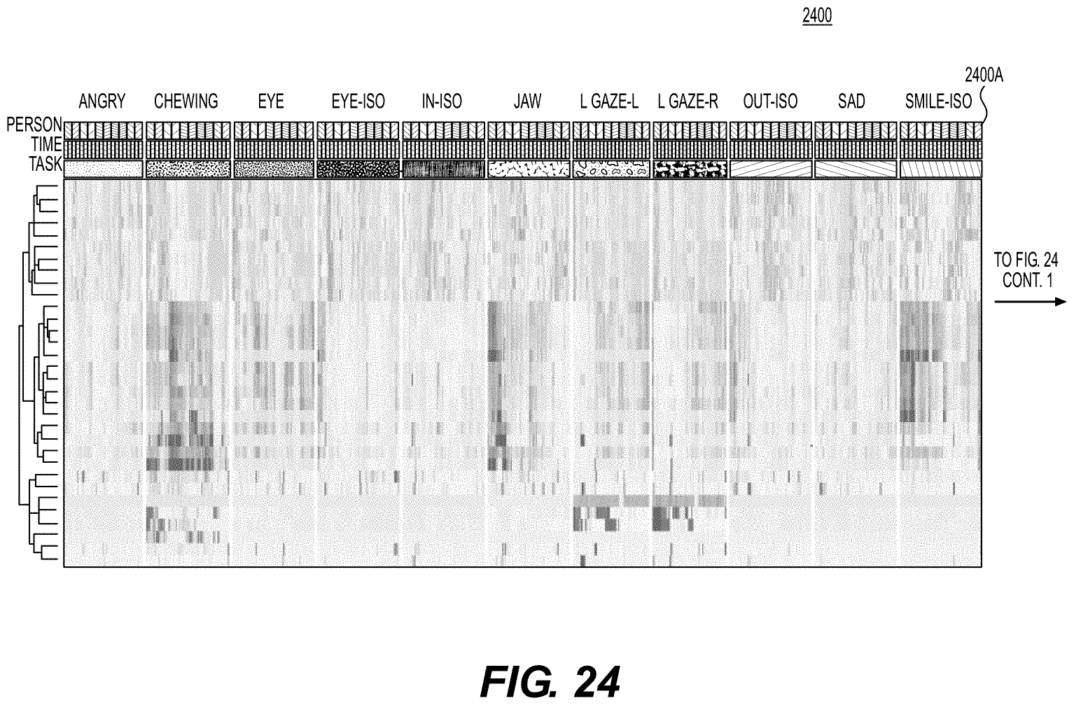

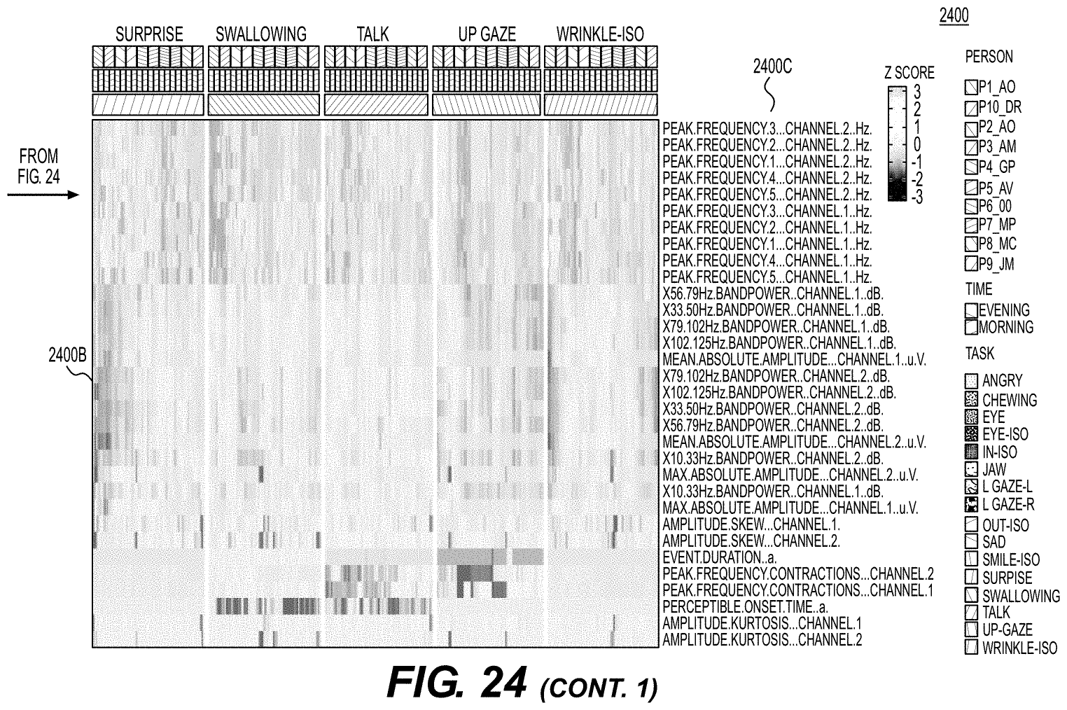

FIG. 24 shows heat map 2400 (including heat maps 2400A and 2400B) similar to chart 904 of FIG. 9 for parameters 2400C. Heat map 2400 shows a heat map of variables and activities that demonstrates qualitative inter subject and inter activity differences. Heat map 2400 shows Z scores for each of a plurality of features, calculated based normalized raw data broken out by tasks (e.g., Angry, Chewing, Eye, Eye-Iso, In-Iso, Jaw, Left gaze-left (L Gaze L), Left gaze-right (L gaze R), Out-Iso, Sad, Smile-Iso, Surprise, Swallowing, Talk, Up Gaze, Wrinkle-Iso, as described further in Table 7), individuals (persons), and time (e.g., morning/evening). The Z scores are distributed using values ranging from −3 to +3 and each value is assigned a color as shown in the legend.

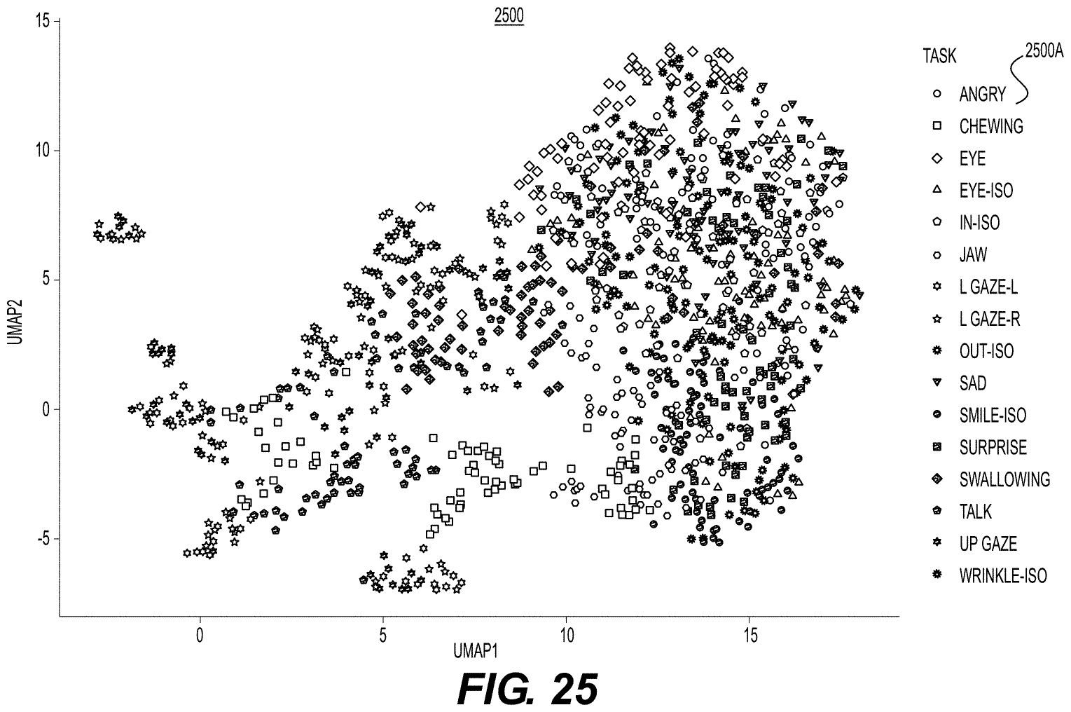

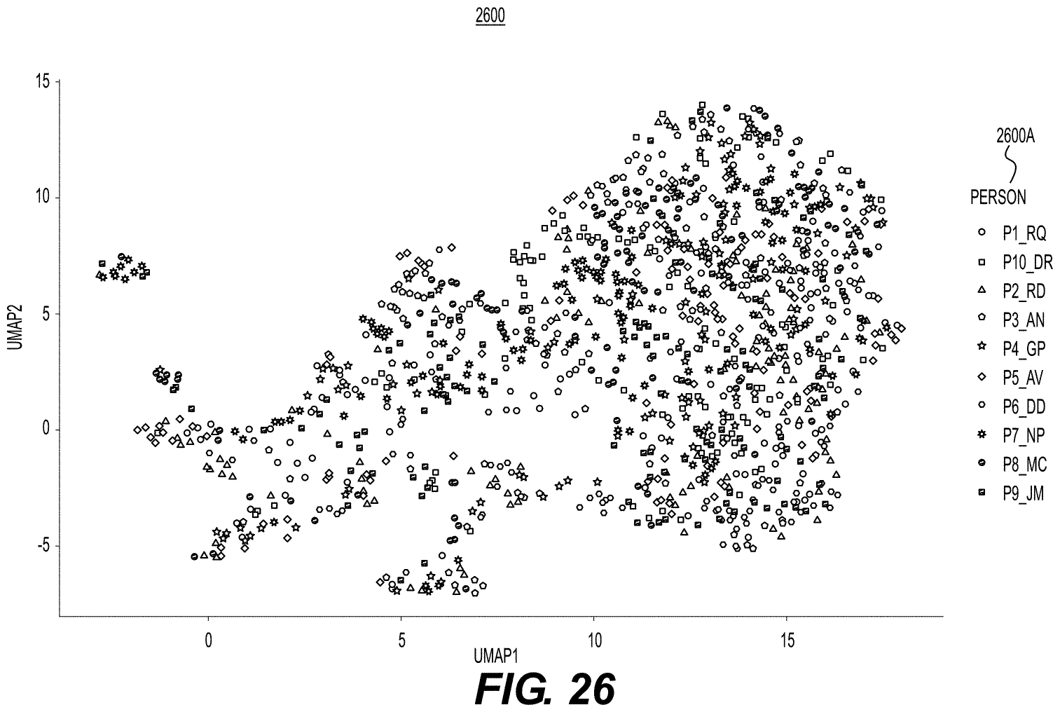

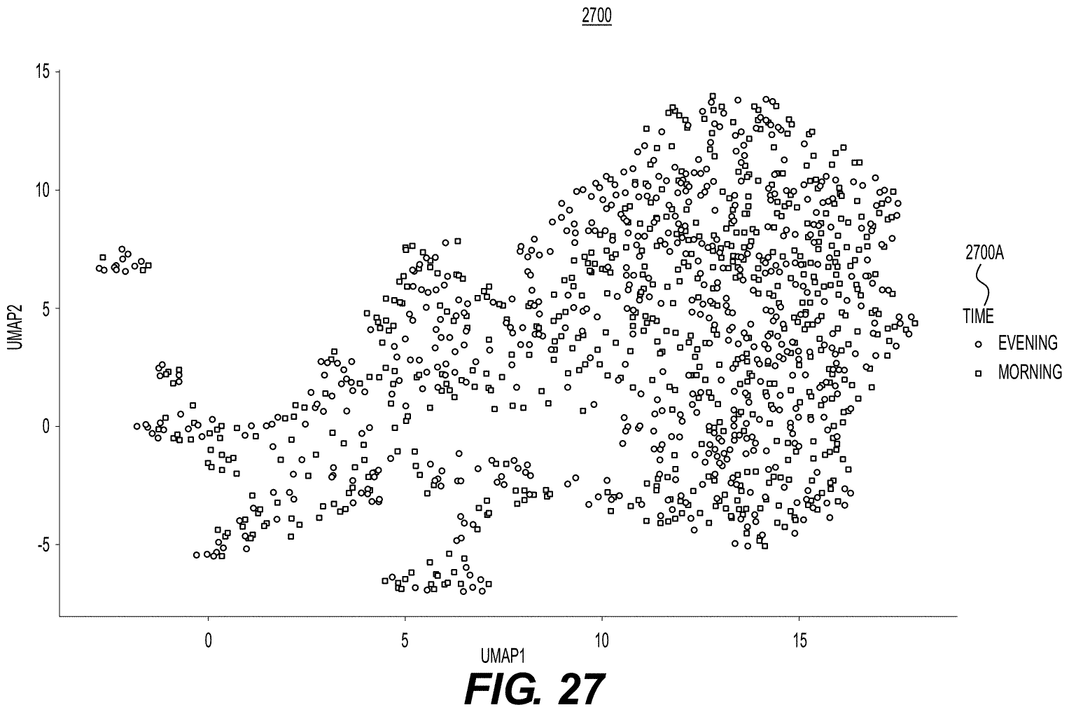

FIG. 25 shows tasks 2500A plotted on a UMAP chart 2500. UMAP Chart 2500 may be generated based on a visual depiction generated by reducing each of a plurality of parameters (e.g., parameters 2400C from FIG. 24) to two values. Accordingly, each trial with multiple iterations (e.g., rows) are reduced to two iterations (e.g., rows) and the results are plotted onto the UMAP. UMAP Chart 2500 shows the resulting data separated out by task 2500A. For example, UMAP chart 2500 could be used to cluster based on each of the tasks 2500A. Similarly, FIG. 26 is a UMAP chart 2600 that is generated using the same data used to generate UMAP chart 2500. UMAP Chart 2600 shows the resulting data separated out by individual 2600A. For example, UMAP chart 2600 could be used to cluster based on each of the individuals 2600A. Similarly, FIG. 27 is a UMAP chart 2700 that is generated using the same data used to generate UMAP chart 2500 and UMAP chart 2600. UMAP Chart 2700 shows the resulting data separated out by time of collection 2700A. For example, UMAP chart 2700 could be used to cluster based on each of the time of collection 2700A.

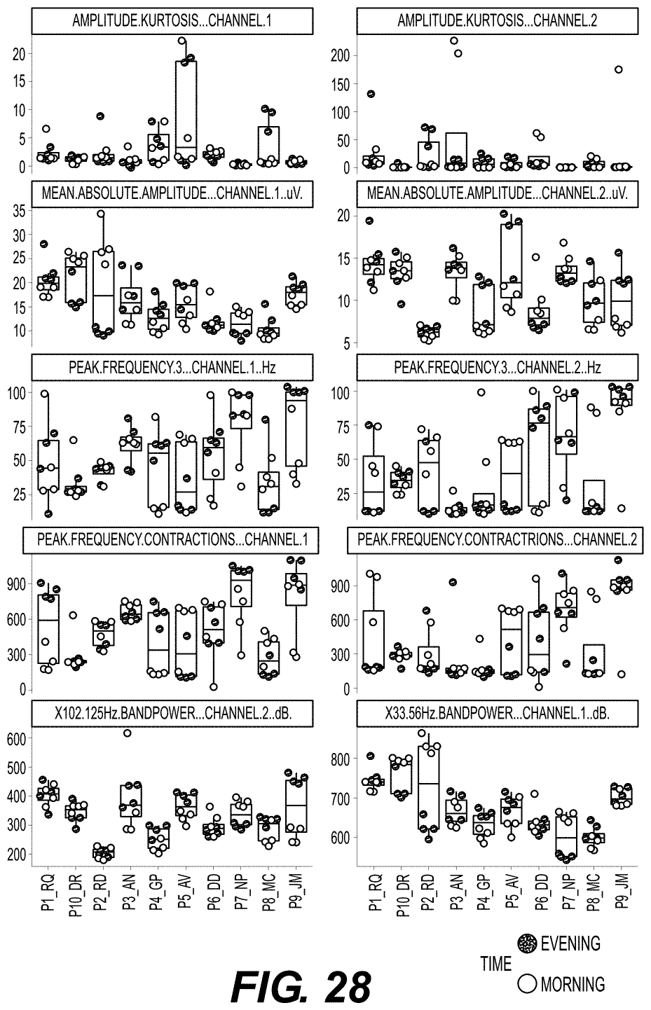

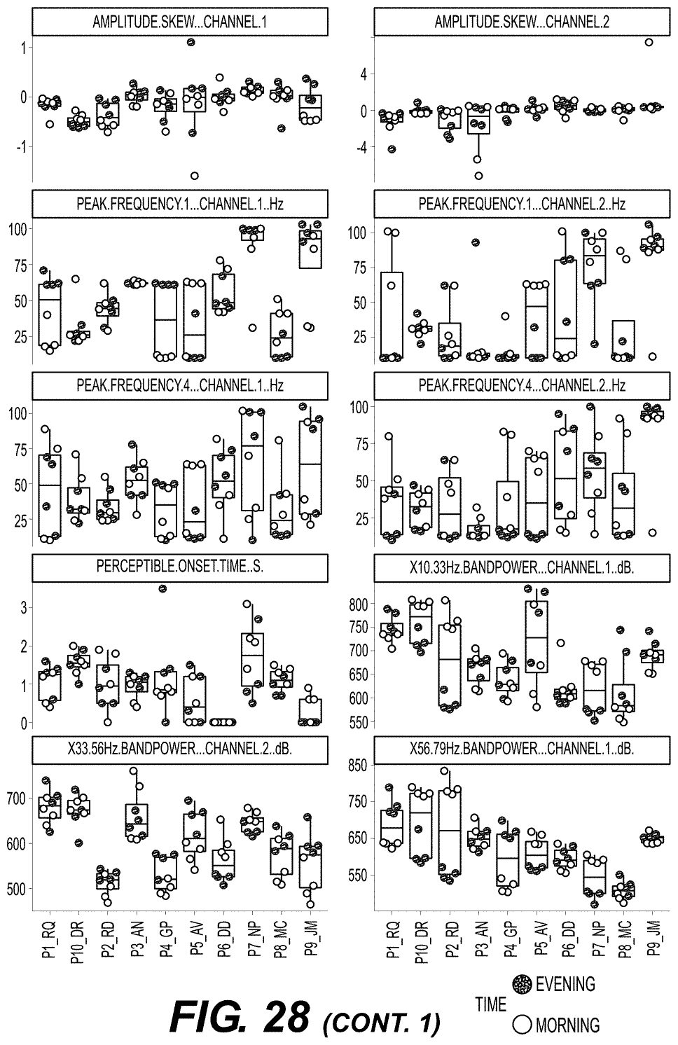

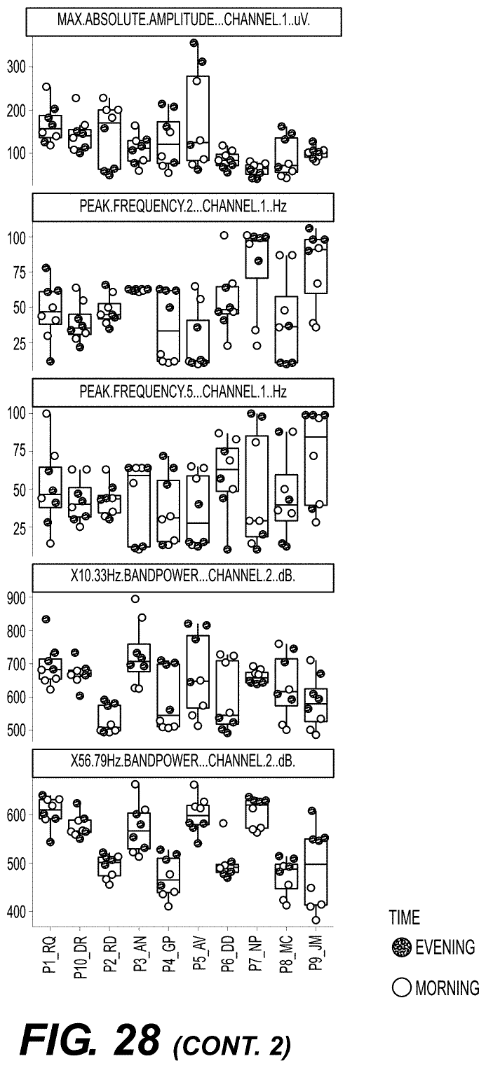

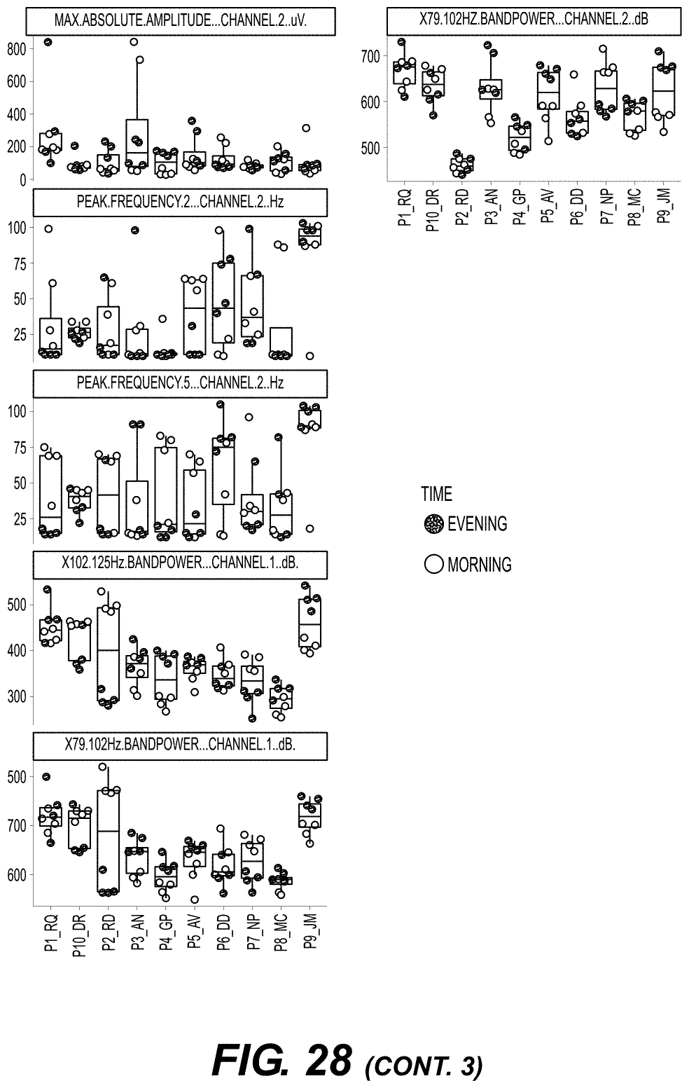

FIG. 28 is similar to FIG. 19 and shows charts 2800 of various features for ten individuals, for a swallowing activity. Thirty-one different data plots are shown in the respective charts. As shown, for each individual, multiple amplitudes are collected for each individual during both morning and evening testing times. The data represented in charts 2800 may be used to generate the Z score based heat map shown in, for example, FIG. 24. According to an implementation, the thirty-one different data plots may each or as a combined data set be an endpoint in a clinical trial. For example, the data represented in the individual data plots or a combined representation of the data may be the clinical output sought as the result of a clinical trial.

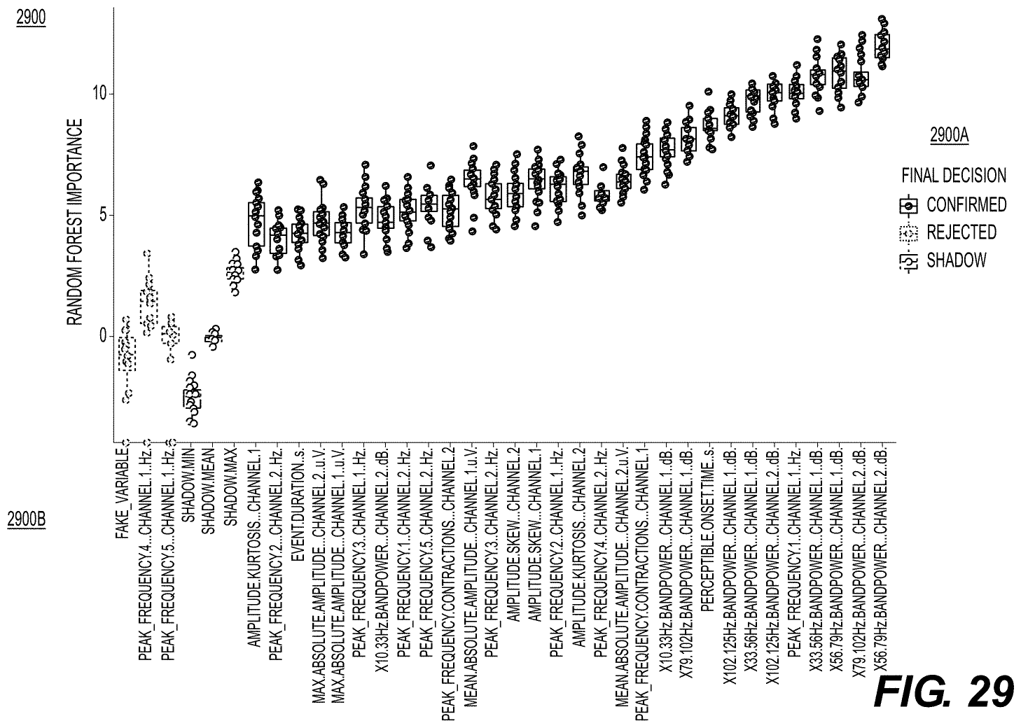

FIG. 29 shows random forest importance values across multiple features 2900B in chart 2900, based on final decisions indicated in legend 2900A. Chart 2900 shows the distribution for each feature 2900B, for a given task. Chart 2900 may be generated using a Boruta feature selection algorithm for feature selection from a dataset. A Boruta algorithm applied may function a wrapper algorithm around Random Forest. The Boruta algorithm adds randomness to the parameter based given data set by creating shuffled copies of all features (e.g., shadow features). Then, a random forest classifier may be trained on the extended data set. A feature importance measure (e.g., mean decrease accuracy) may be applied to evaluate the importance of each feature where higher means more important. At each iteration, a check may be conducted for whether a real feature has a higher importance than the best of its shadow features (e.g., whether the feature has a higher Z score than the maximum Z score of its shadow features) and features which are deemed highly unimportant (e.g., above a Boruta threshold) may be discarded for identifying a clinical output. Clinical trials may require use of features that meet or exceed the Boruta threshold). The algorithm may complete a cycle either when all features are confirmed or rejected or when specified limit of random forest runs is reached.

The random forest importance values across multiple features shown in FIG. 29 may be used to determine how important each feature is for classifying a certain activity. Features that are highly important (e.g., above a Boruta threshold) may be designated as clinically relevant features 50. As an example, the results shown in FIG. 29 may be used to reduce a number of features based on comparing real values of a given feature to shuffled values (generated based on duplication). A given feature that adds more value to a classification (e.g., determined based on removing the feature and determining how much information is lost) may receive a higher score. Machine learning may be used to for permutation based score identification. A given feature may be determined more clinically relevant for a given task than another feature. Additionally, or alternatively, an end point in a clinical trial may be determined when the removal of any remaining features decreases an ability to classify information by a given threshold. A random forest score may be based on the result of the Boruta model, where features with higher impact are given a higher score. Extracted features having a random forest score at or above a random forest threshold are identified as clinically relevant features.

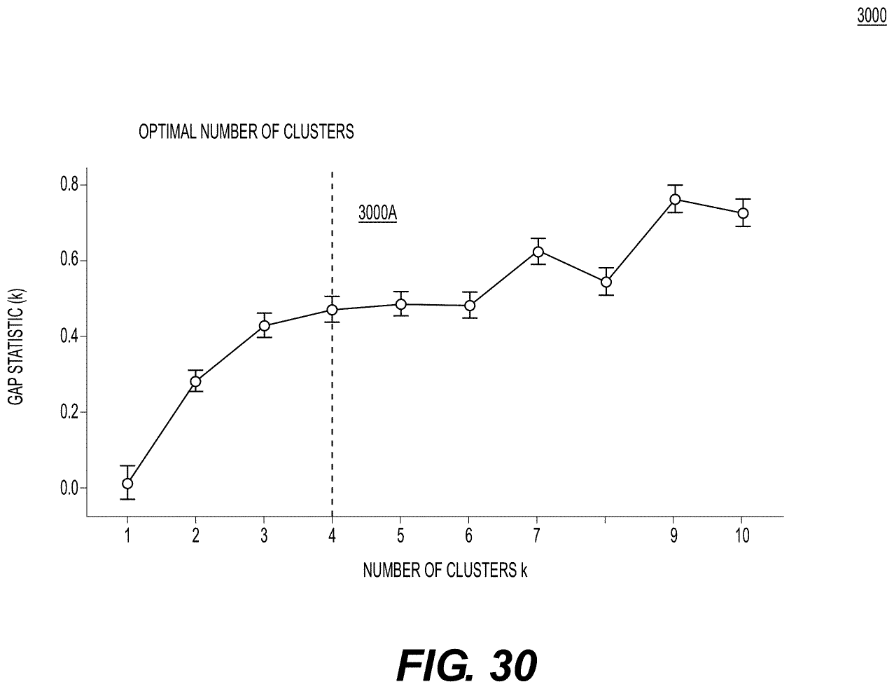

FIG. 30 shows a chart 3000 including gap statistics. Using gap statistics (e.g., a k value), an optimal number of clusters for a given data set may be determined. Chart 3000 may be related to, for example, the spearman correlation plot of FIG. 10, where the spearman may be used to identify a number of clusters based on the available signal based data. As shown, an optimal number of clusters 3000A based on the data presented in chart 3000 is 4, such that having 4 clusters given the respective data, provides a threshold balance between differences among the features in the clusters and a number of clusters.

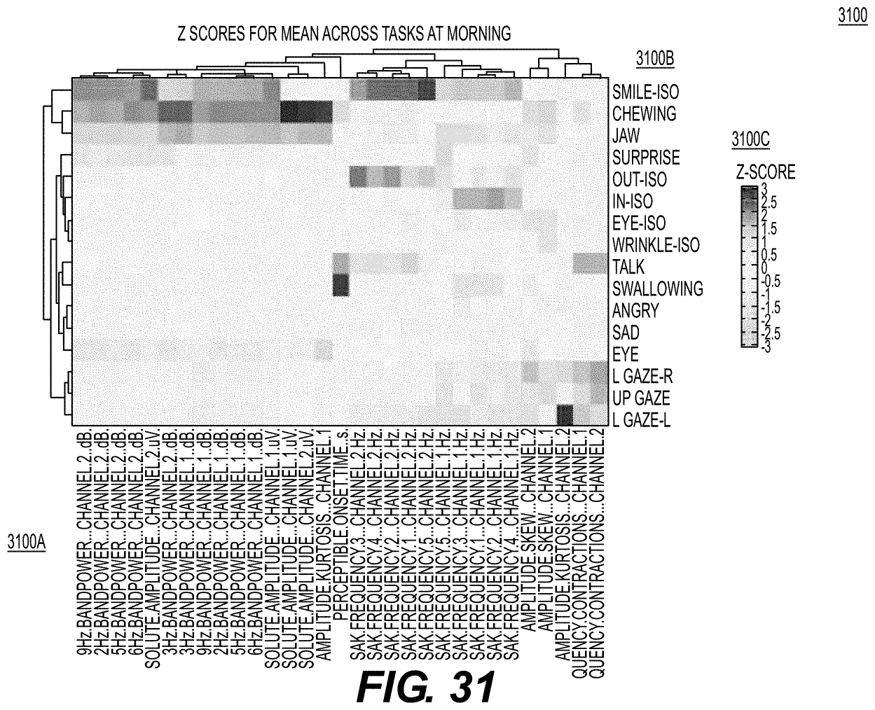

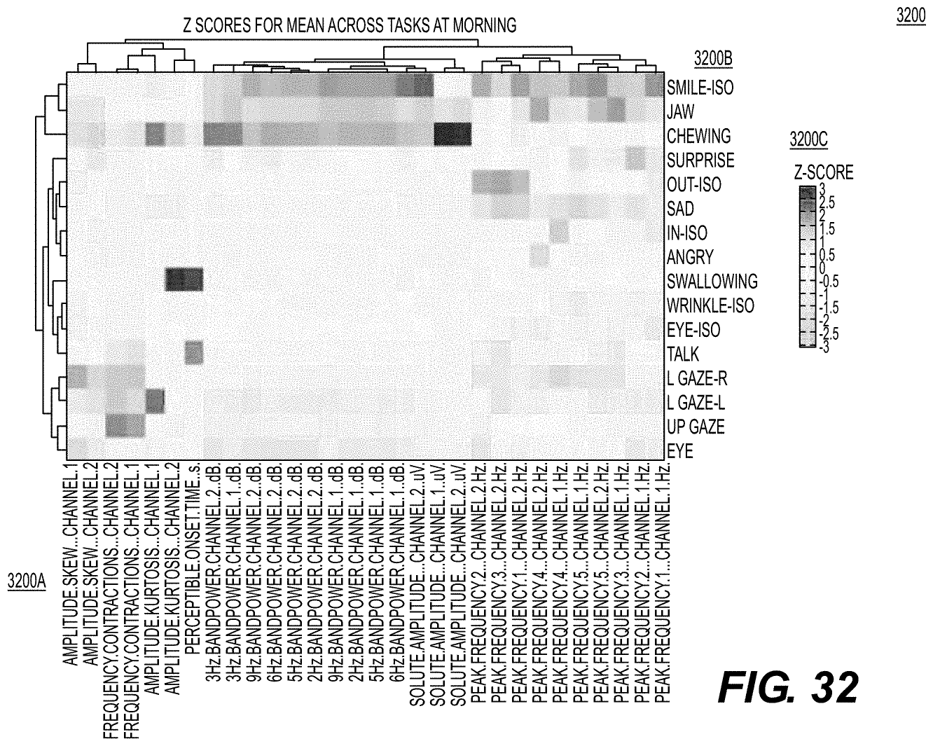

FIG. 31 shows Z scores, via heat map 3100, across tasks during morning collections. The Z scores are for each feature 3100A per task 3100B, based on legend 3100C, which range in values from −3 to +3 and assigned a color. FIG. 32 shows Z scores, via heat map 3200, across tasks during evening collections. The Z scores are for each feature 3200A per task 3200B, based on legend 3200C, which range in values from −3 to +3 and assigned a color. As disclosed herein, the Z scores shown on a heat map (e.g., heat map 3100 and/or 3200) are normalized relative to respective ranges such as a high value within a range of possible values corresponds to a high Z score and a low value within a range of possible values corresponds to a low Z score.

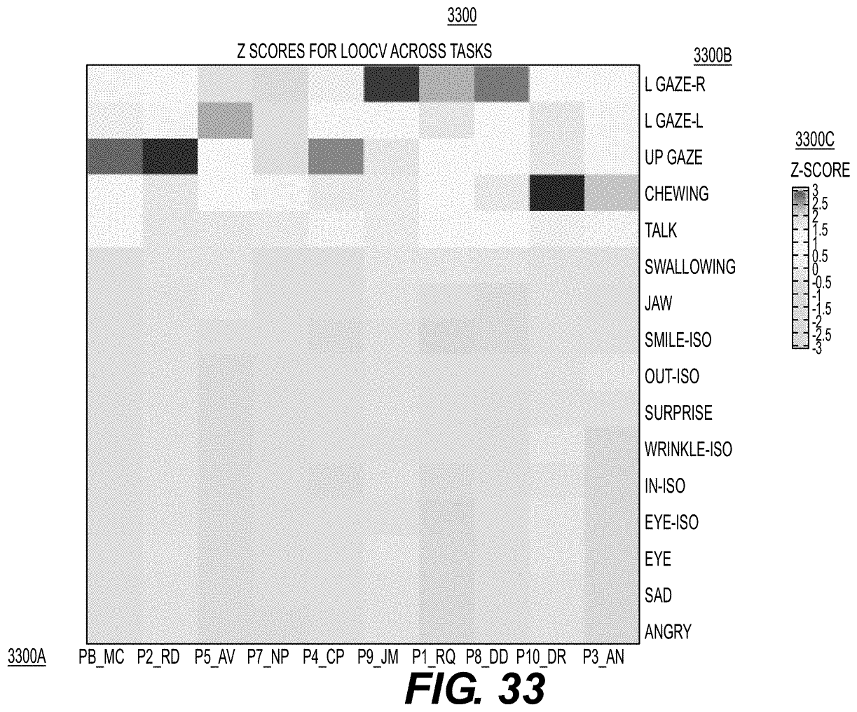

FIG. 33 shows Z scores, on heat map 3300, for LOOCV across tasks. The heat map 3300 of FIG. 33 is generated by a procedure used to estimate the performance of machine learning algorithms. The data is provided for individuals 3300A and tasks 3300B, based on legend 3300C, which range in values from −3 to +3 and assigned a color. In LOOCV, a number of folds may equal the number of instances in a data set. Thus, the learning algorithm may be applied once for each instance, using all other instances as a training set and using the selected instance as a single-item test set. For example, to generate the heat map 3300, an individual's data may be removed from a training set of a predictive machine learning algorithm, and the remaining data may be used to train the algorithm. The algorithm may then be used predict the removed individual's data. The Z scores shown in heat map 3300 may be generated based on how the prediction compares to the actual data and/or how well the prediction identifies a given task for an individual.

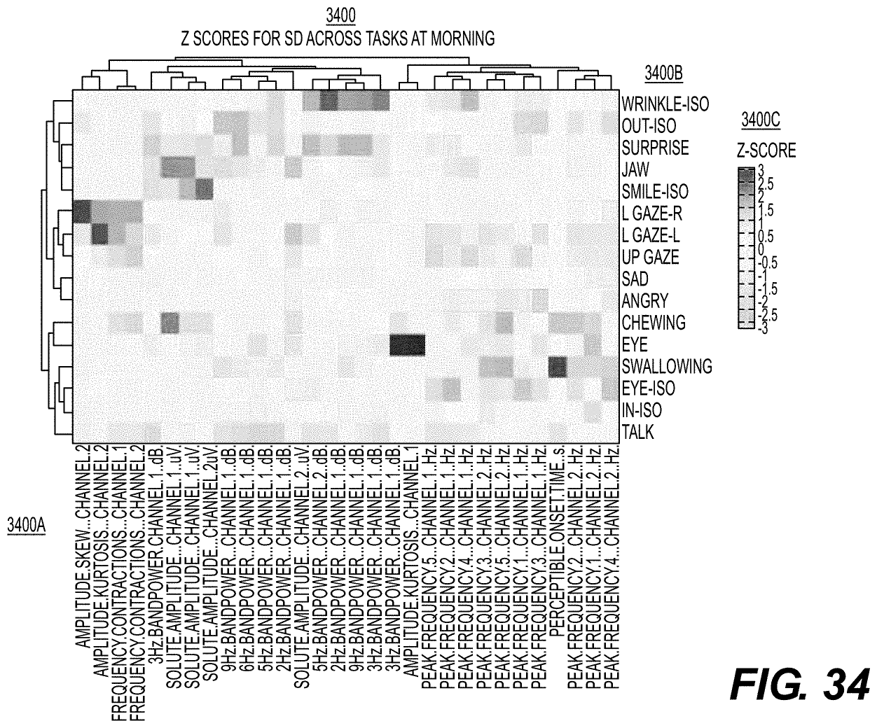

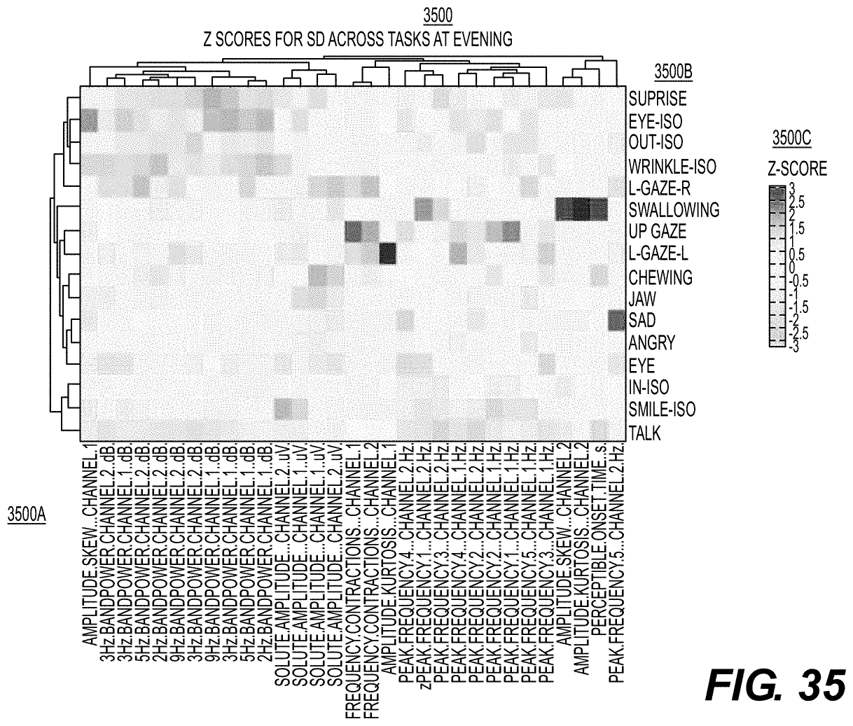

FIG. 34 shows Z scores in a heat map 3400 for standard deviation (SD) during morning collections. The heat map 3400 shows the Z score of standard deviation for features 3400A during tasks 3400B, based on legend 3400C, which range in values from −3 to +3 and assigned a color. FIG. 35 shows Z scores in a heat map 3500 for SD during evening collections. The heat map 3500 shows the Z score of standard deviation for features 3500A during tasks 3500B, based on legend 3500C, which range in values from −3 to +3 and assigned a color. The standard deviations indicate the amount of variability in the feature data such that high standard deviation may indicate less reliability whereas a low standard deviation may indicate greater reliability.

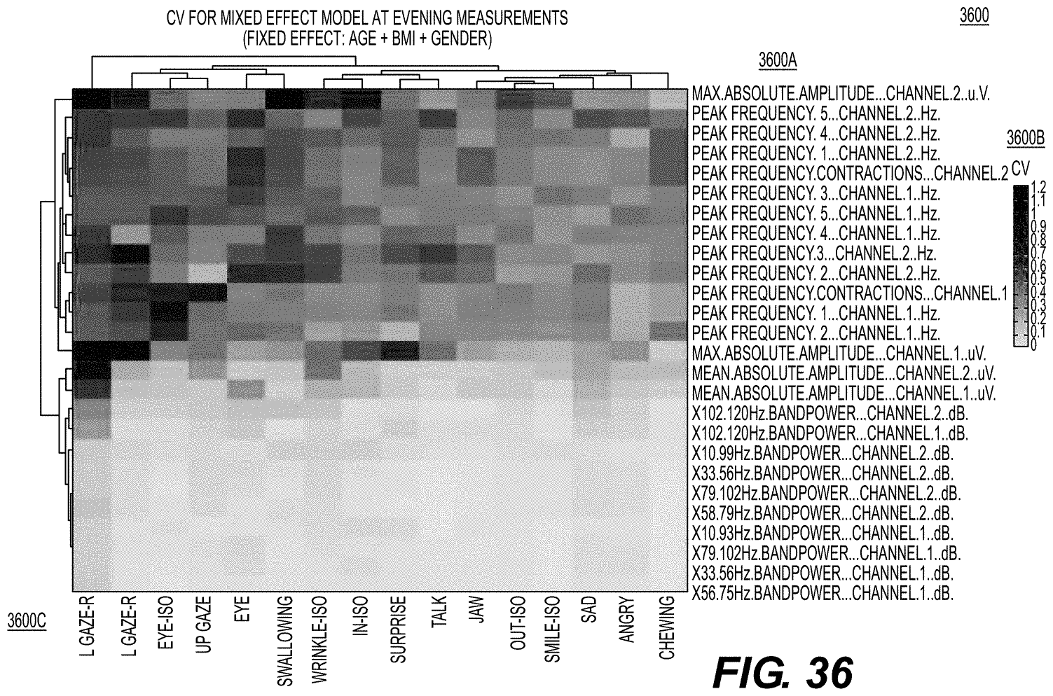

FIG. 36 shows a CV with heat map 3600 for a mixed effect model for evening collections. FIG. 36 is similar to FIG. 22. FIG. 36 shows CVs based on a mixed effect model using morning measurements with fixed effects age, BMI, and gender. The CV heat map 3600 shows the CV values for features 3600A for tasks 3600C based on legend 3600B, which range in values from 0 to 1.2 and assigned a color. The results of CV heat map 3600 may be used to identify which features will be reliable (e.g., low variance) for determining a clinical output (e.g., disease designation. For example, a lower CV for a given feature and action may indicate that the given feature can be repeated in a reliable manner (e.g., meets a CV reliability threshold) for multiple tests. A clinical trial may require that features used for the trial meet such a CV reliability threshold.

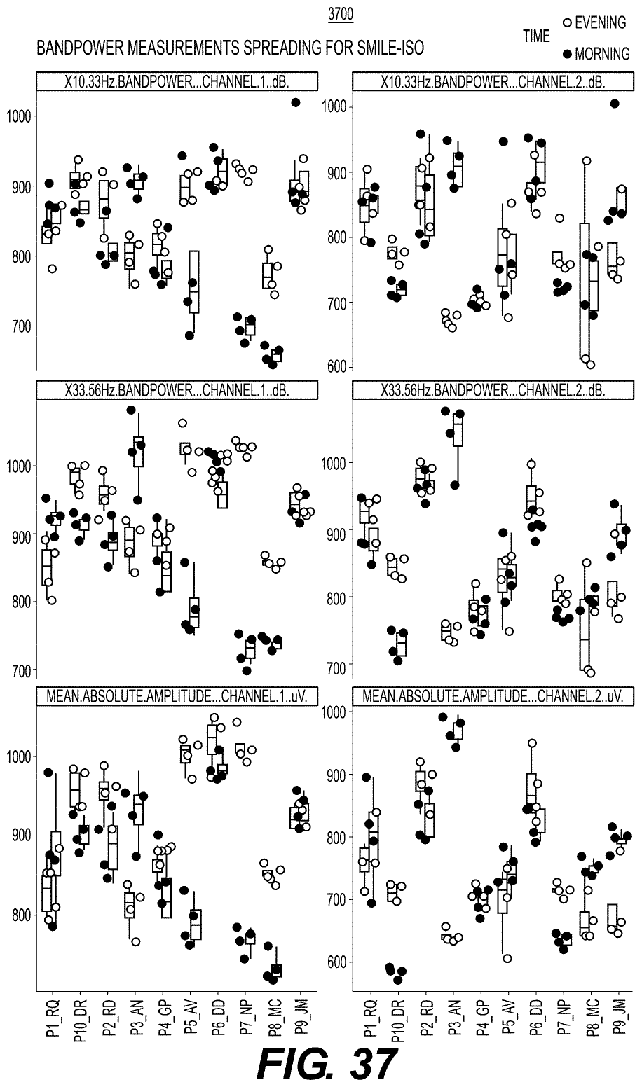

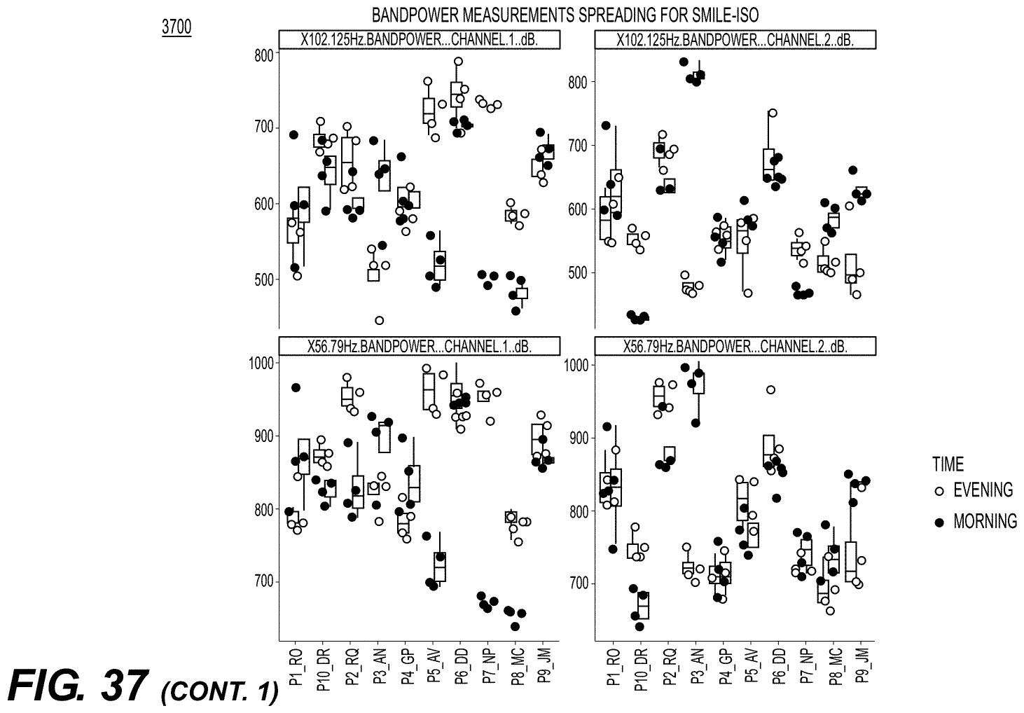

FIG. 37 shows band-power measurements spreads 3700 for smile collections. Spreads 3700 are similar to those calculated for swallowing in FIG. 28. Spreads 3700 show various features for ten individuals, for a smiling activity. Ten different data plots are shown in the respective spreads. As shown, for each individual, multiple amplitudes are collected for each individual during both morning and evening testing times. The data represented in spreads 3700 may be used to generate the Z score based heat map shown in, for example, FIG. 24.

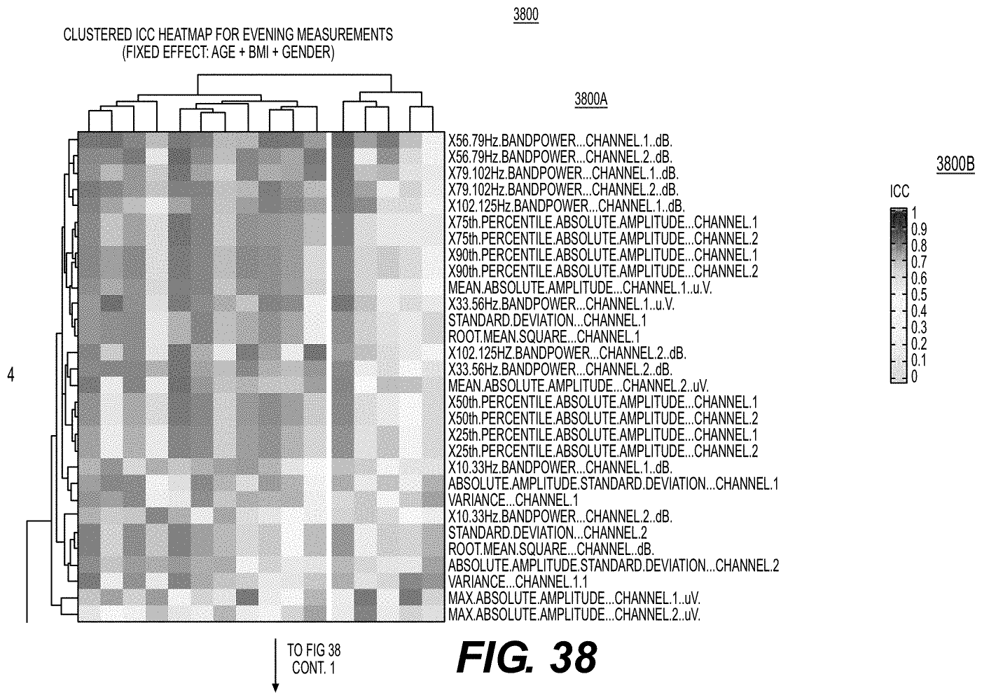

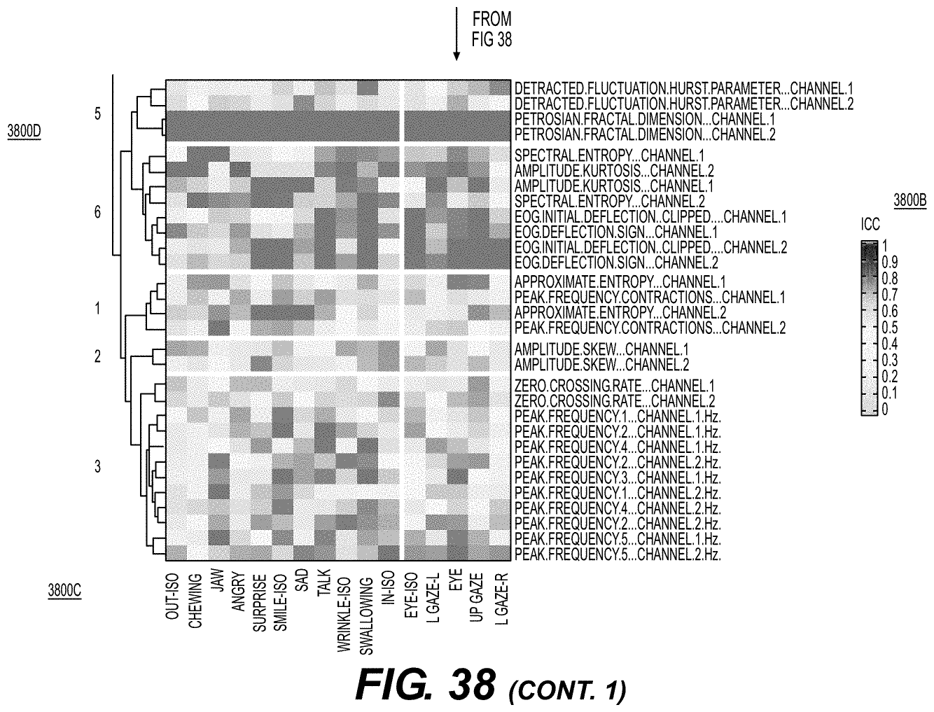

FIG. 38 shows a cluster ICC heat map 3800 for evening measurements, with fixed effects age, BMI, and gender. Heat map 3800 is based on features 3800A for tasks 3800C based on legend 3800B, which range in values from 0 to 1 and assigned a color. The ICC measurements shown in heat map 3800 based indicate that if the same measurement is calculated for a given person across multiple collections, how similar are the results for the given person. The result identifies the correlation for the same individual with the individual's data. Accordingly, the heat map 3800 indicates the test and re-test reliability. A higher ICC value (e.g., 1) indicates that for a given individual, the difference across multiple tests, is low, so the data for the individual correlates with itself. A lower ICC value (e.g., 0) indicates that for a given individual, the difference across multiple tests, is high. The heat map 3800 also indicates which features meet an ICC threshold such that a feature associated with a higher ICC value (e.g., 1) across multiple subjects may be used for a clinical trial as it reliably provides data for individuals. As shown at 3800D, the various ICC correlation values may be clustered (e.g., in 6 clusters in this example).

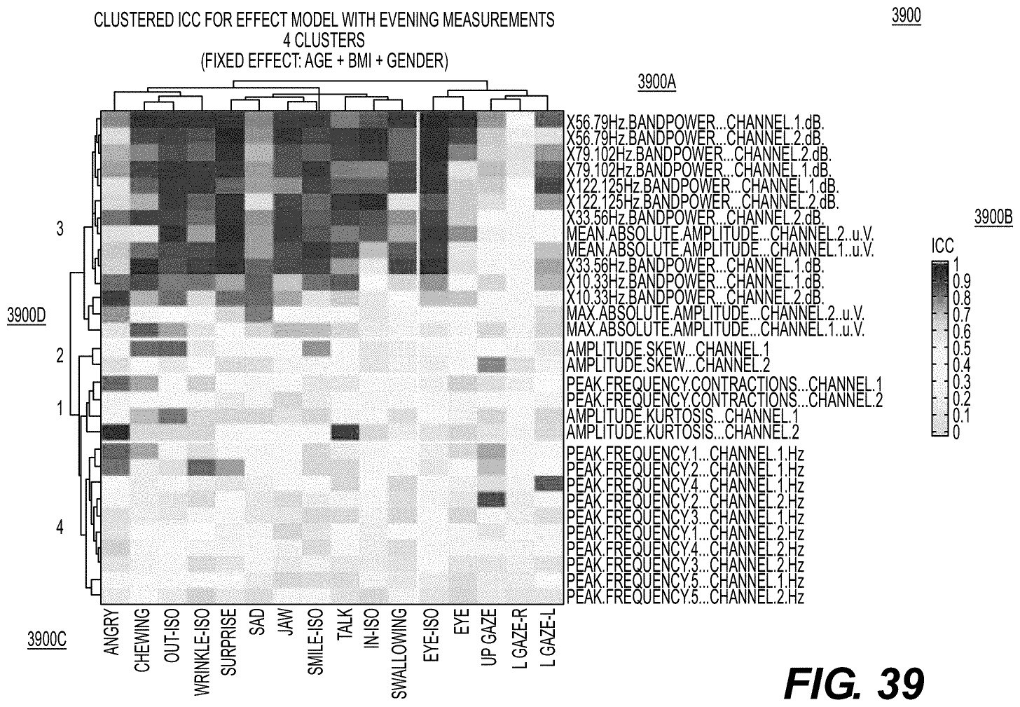

FIG. 39 shows another cluster ICC heat map 3900 for evening measurements, with fixed effects age, BMI, and gender. Heat map 3900 is based on features 3900A for tasks 3900C based on legend 3900B, which range in values from 0 to 1 and assigned a color. The ICC measurements shown in heat map 3900 based indicate that if the same measurement is calculated for a given person across multiple collections, how similar are the results for the given person. The result identifies the correlation for the same individual with the individual's data. Accordingly, the heat map 3900 indicates the test and re-test reliability. A higher ICC value (e.g., 1) indicates that for a given individual, the difference across multiple tests, is low, so the data for the individual correlates with itself. A lower ICC value (e.g., 0) indicates that for a given individual, the difference across multiple tests, is high. The heat map 3900 also indicates which features meet an ICC threshold such that a feature associated with a higher ICC value (e.g., 1) across multiple subjects may be used for a clinical trial as it reliably provides data for individuals. As shown at 3900D, the various ICC correlation values may be clustered (e.g., in 4 clusters in this example).

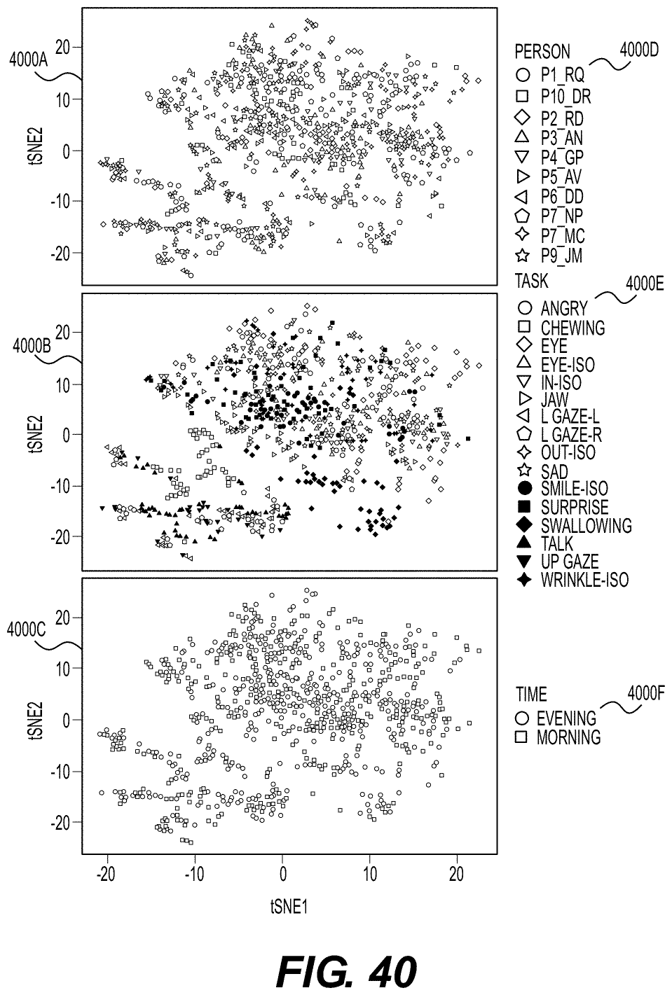

FIG. 40 shows t-distributed stochastic neighbor embedding (t-SNE) charts 4000A, 4000B, and 4000C for individuals, tasks, and time. Chart 4000A corresponds to reduced data based on individuals 4000D, chart 4000B corresponds to reduced data based on tasks 4000E, and chart 4000C corresponds to reduced collection times 4000F. The charts 4000A, 4000B, and 4000C may be generated based on an algorithm to reduce the number of datasets to two dimensions. Charts 4000A, 4000B, and 4000C may be used to compare how similar or dissimilar the respective dataset is (e.g., how variable the data associated with each individual in 4000D is, as shown in 4000A). Comparing the charts 4000A, 4000B, and 4000C may indicate the contribution from variance based on a given parameter (e.g., across individuals 4000D, across tasks 4000E, and/or across collection time 4000F).

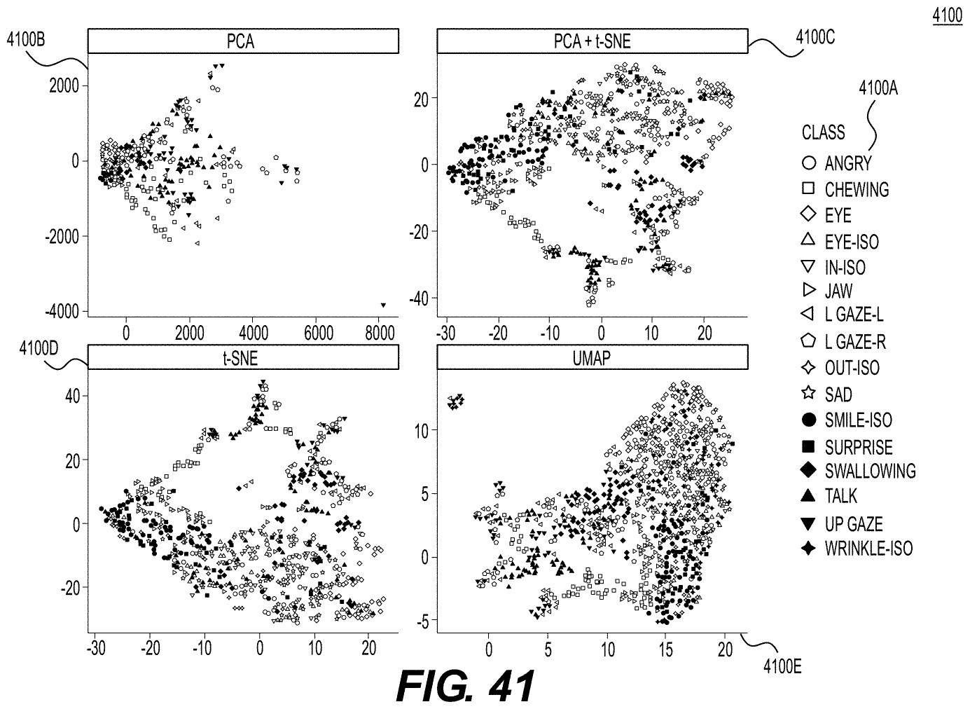

FIG. 41 shows various charts 4100 for visualization of data reduced to two dimensions, based on tasks 4100A. Chart 4100B is based on principal component analysis (PCA), where principal components of a collection of points in a real coordinate space are a sequence of p unit vectors, where the i-th vector is the direction of a line that best fits the data while being orthogonal to the first i−1 vectors. Chart 4100C is based on both PCA and t-SNE reduction. Chart 4100D is based on t-SNE reduction. Chart 4100E is based on UMAP reduction.

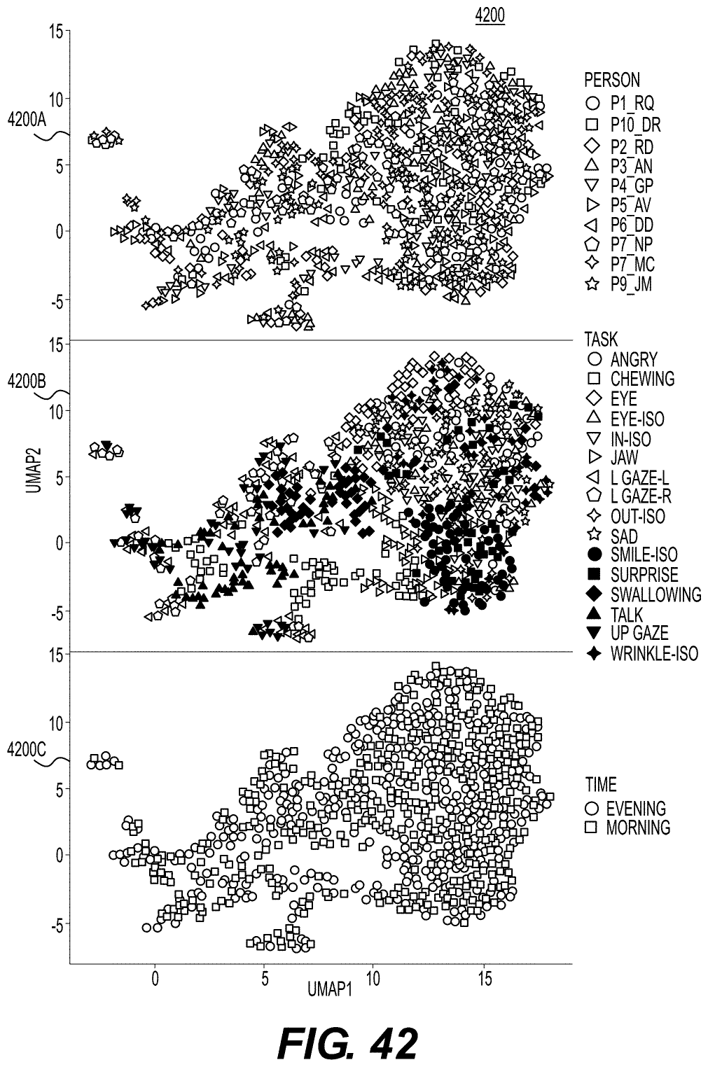

FIG. 42 shows UMAP charts 4200 for individuals in chart 4200A, tasks in chart 4200B, and time in chart 4200C. Charts 4200A, 4200B, and 4200C may be similar to those provide din FIGS. 25-27. Chart 4200A may show tasks plotted using a UMAP reduction. Chart 4200A shows a visual depiction generated by reducing each of a plurality of parameters (e.g., parameters 2400C from FIG. 24) to two values. Accordingly, each trial with multiple iterations (e.g., rows) are reduced to two iterations (e.g., rows) and the results are plotted onto the UMAP chart. Chart 4200A shows the resulting data separated out by task. For example, chart 4200A could be used to cluster based on each of the tasks. Similarly, chart 4200B is generated using the same data used to generate Chart 4200A. Chart 4200B shows the resulting data separated out by individual. For example, Chart 4200B could be used to cluster based on each of the individuals. Similarly, chart 4200C is generated using the same data used to generate chart 4200A or 4200B. Chart 4200C shows the resulting data separated out by time of collection. For example, chart 4200C could be used to cluster based on each of the times.

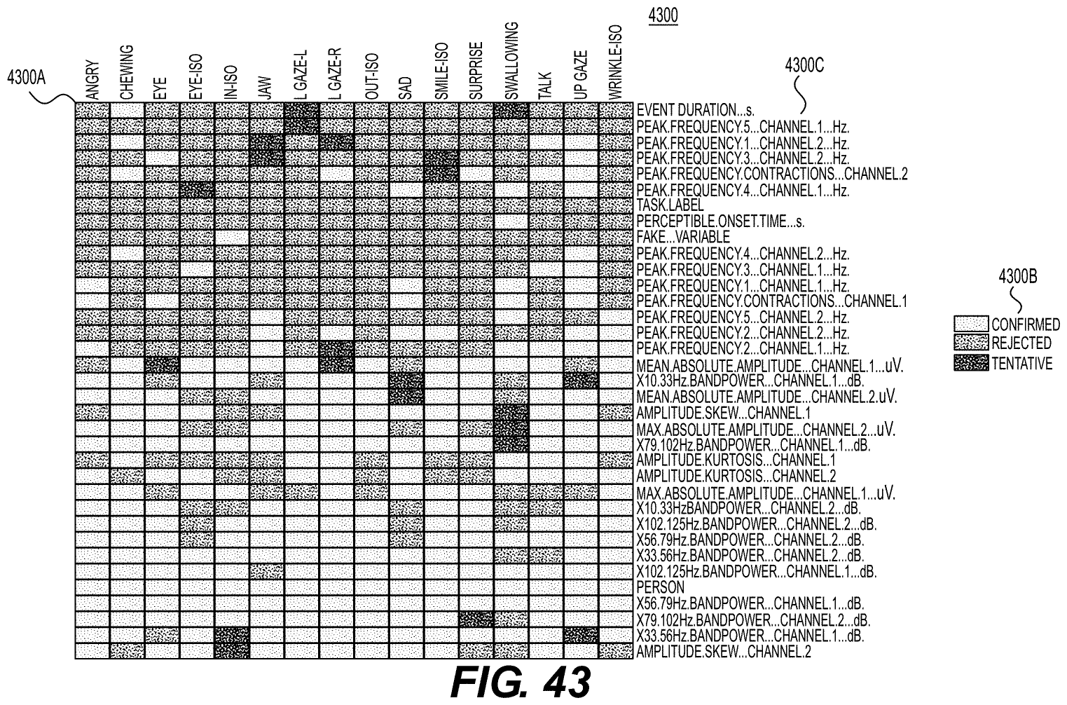

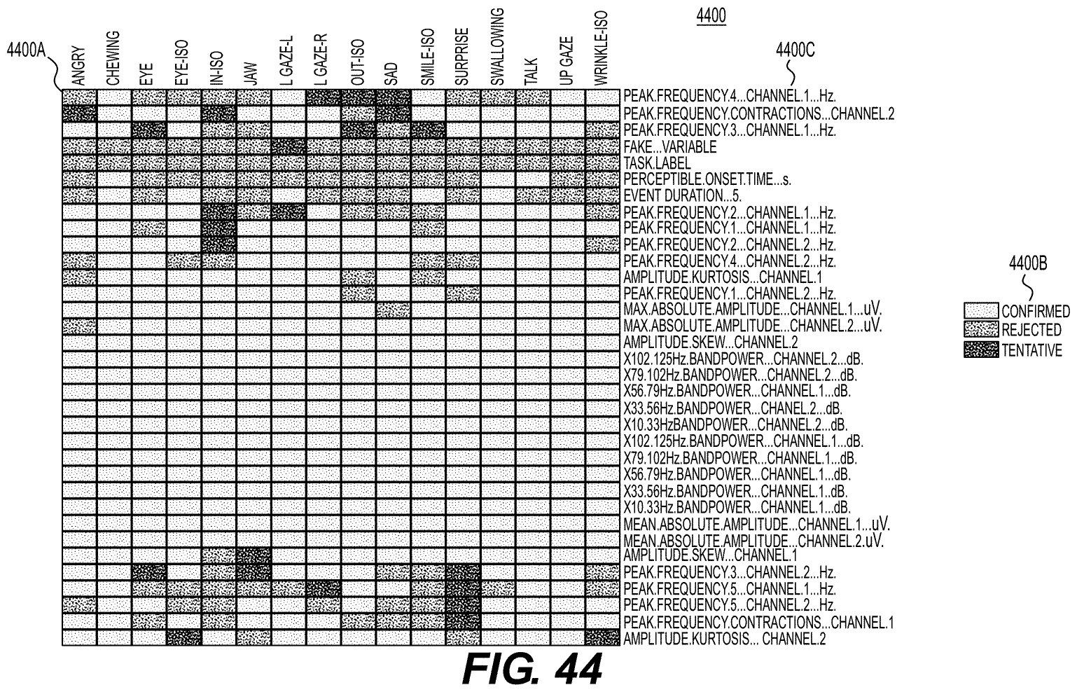

FIGS. 43 and 44 show confirmation results in charts 4300 and 4400, for different tasks across various channels. The data provided in charts 4300 and 4400 may be generated based on the Boruda analysis (e.g., feature importance) discussed in FIG. 29. Charts 4300 and 400 show confirmation results 4300B and 4400B for tasks 4300A and 4400A for parameters 4300C and 4400C. FIG. 29, for example, shows the analysis results for a single task whereas FIGS. 43 and 44 show analysis results for multiple tasks. Charts 4300 and 4400 indicate whether given data (e.g., results 4300B and 4400B for tasks 4300A and 4400A for parameters 4300C and 4400C) is clinically relevant in identifying a clinical outcome. Relevant data is indicated as confirmed, whereas irrelevant data is rejected. Data that does not meet a relevant threshold or irrelevant threshold is designated as tentative. A clinical trial may require that parameters used for the trail met the relevance threshold for confirmation, for use in the clinical trial to determine the clinical outcome.

### Example 2

This example provides a protocol used to evaluate the ability of a signal capture device 10 to facilitate a determination clinically relevant features based on the signal capture device 10's capabilities and based on clinical outcomes. Although the example provides specific applications of the techniques disclosed herein, it will be understood that additional applications of techniques may be implemented.

In accordance with the techniques disclosed herein, as an initial step to developing a digital assessment of neuromuscular disorders, a study was conducted to determine whether a biometric sensor device could be utilized to objectively measure facial muscle and eye movements intended to be representative of Performance Outcome Assessments (PerfOs) with tasks designed to model clinical PerfOs, referred to as mock-PerfO activities. The specific aims of this study were: to determine whether the biometric sensor device raw EMG, EOG, and EEG signals could be processed to extract features describing these waveforms; to determine a biometric sensor device feature data quality, test re-test reliability, and statistical properties; to determine whether features derived from the biometric sensor device could be used to determine the difference between various facial muscle and eye movement activities; and, to determine what features and feature types are important for mock-PerfO activity level classification.

It will be understood that the clinical outcome to be tested in this example is identifying what features and feature types are important for mock-PerfO activity level classification, based on the biometric sensor device.

The biometric sensor device used in this example is behind-the-ear wearable originally developed to measure cognitive function. Since the biometric sensor device measures, e.g., electroencephalography (EEG), electromyography (EMG), and electrooculography (EOG) data, it may also have the potential to objectively quantify facial muscle and eye movement activities relevant in the assessment of neuromuscular disorders.

A total of N=10 healthy volunteers participated in the study. Each study participant performed 16 mock-PerfOs activities, including talking, chewing, swallowing, eye closure, gazing in different directions, puffing cheeks, chewing an apple, and making various facial expressions. Each activity was repeated four times in the morning and four times at night. A total of 161 summary features were extracted from the EEG, EMG, and EOG bio-sensor data. Feature vectors were used as input to machine learning models to classify the mock-PerfO activities, and model performance was evaluated on a held-out test set. Additionally, a convolutional neural network (CNN) was used to classify low-level representations of the raw bio-sensor data for each task, and model performance was correspondingly evaluated and compared directly to feature classification performance.

The model's prediction accuracy on the biometric sensor device's classification ability was quantitatively assessed. Study results indicate that the biometric sensor device tested can potentially quantify different aspects of facial and eye movements and may be used to differentiate mock-PerfO activities. Specially, the identified clinically relevant features indicated that the biometric sensor device tested was found to differentiate talking, chewing, and swallowing tasks from other tasks with observed F1 scores >0.9. While EMG features contribute to classification accuracy for all tasks, EOG features are important for classifying gaze tasks. It was found that analysis with summary features outperformed a CNN for activity classification.

As further discussed herein, it was determined that the biometric sensor device met clinical thresholds such that it may be used to measure cranial muscle activity relevant for neuromuscular disorder assessment. Classification performance of mock-PerfO activities with summary features enables a strategy for detecting disease-specific signals relative to controls, as well as the monitoring of intra-subject treatment responses.

Facial/cranial and eye movement dysfunction is an important feature of several neurological disorders that affect multiple levels of the neuraxis. Examples include outright facial weakness due to facial nerve palsy or stroke, diplopia, ptosis, and dysphagia caused by neuromuscular disorders such as myasthenia gravis, dystonia, complex extraocular movement deficits, hypomimia, and dysphagia caused by parkinsonian (and other neurodegenerative) conditions.

As discussed herein, clinical assessment of these symptoms remains a challenge in medicine and clinical research. Existing clinical assessments, such as clinician-reported outcomes (ClinROs) or patient-reported outcomes (PROs) may require the patient to frequently visit sites, primarily rely on subjective measures, and may not necessarily reflect a patient's condition(s) in the real world. Importantly, patient symptoms can be intermittent and vary throughout the day, making reliable assessment difficult. Finally, they can be variable from patient-to-patient depending on their adaptations to increasing muscle weakness. For example, chart 500 of FIG. 5 highlights the subjective nature of physician scores 502 based on observation. Such subjective scoring can cause patient-to-patient as well as provider-to-provider variability.

While there are tools that exist to perform quantitative analysis of cranial muscle function, these tools have significant limitations. For example, facial movements can be measured with video-based technologies using either static images or video capture. Surface EMG, which records the electrical movements of facial muscles, can also be used either alone or in combination with video-based methods. Small studies have suggested that EOG, which measures electrical potential from the front to the back of the eye, can detect differences between parkinsonian patients and controls. Screen-based trackers and wearable glasses have been used to monitor extraocular movements and upper cranial activity (e.g., blinking). Together in their current application, these approaches can be cumbersome, difficult to implement, and most importantly, capture facial movements for brief periods of time in an artificial setting.

Accordingly, techniques disclosed herein are advantageous such that they provide an opportunity to identify and/or develop novel non-invasive approaches to measure individual attributes (e.g., cranial symptoms of neuromuscular and neurodegenerative disorders) to address problems in key patient populations. The techniques disclosed herein serve to support diagnostic and disease progression assessments by clinicians, and also outcomes assessment in clinical research. If such approaches can leverage wearable sensing technology, they may be able to address the challenges of existing clinical sensors that are limited for use in highly controlled settings, as opposed to more naturalistic environments (e.g., at home).

The tested biometric sensor device is a behind-the ear device developed to measure neural and physiological processes. Electrophysiological signals are acquired at 250 Hz via four re-usable electrodes fabricated from a conductive silicon material. Electrodes of the device are positioned at scalp locations directly above the left and right ears and on left and right mastoid processes, yielding raw bio-signal data analogous to that which could be acquired at Electroencephalography (EEG) reference locations T3, T4, M1, and M2 of the 10-20 electrode placement positions, EEG being a measurement of surface brain wave function. This electrode configuration also enables high fidelity acquisition of EMG activity from activation of the temporalis and surrounding muscle groups, and EOG signals yielded by eye deflections.

Whereas traditional clinical assessment using biophysiological data may be invasive, expensive, and time consuming, the tested biometric sensor device is purposed to offer high fidelity data acquisition and processing to the general population. The EMG, EEG, and EOG signals monitored with the tested biometric sensor device were used for the detection and evaluation of a wide variety of physiological phenomena, such as sleep monitoring, microsleep detection, and acute postoperative pain quantification. Based on identification of clinically relevant features 50 using the biometric sensor device (signal capture device 10), it was determined that the device has the potential to support outcome assessment for neuromuscular disorders by objectively quantifying facial muscle and eye movement tasks through capturing and analyzing bio-signal data. The objective quantification quality was evaluated by generated extracted features 40 from distinct signals 30 collected via the biometric sensor device. The distinct signals 30 were generated using a signal manipulation module 20 that received signals from the biometric sensor device. Techniques disclosed herein were applied to identify clinically relevant features 50 from the extracted features 40. The clinically relevant features 50 met threshold values for clinical outcomes including diagnosis and/or treatment of neuromuscular disorders.

A significant challenge addressed by the techniques disclosed herein is that unprocessed bio-signal data is inherently noisy due to several factors, e.g. participants move during clinical assessments, there may be perturbations in electrode-skin contact, and/or there are artifacts from cardiac activity, or the like. Additionally, similar factors naturally induce artifacts in the acquired signal data; EEG, EMG, and EOG signals overlap in typical frequency ranges, making direct separation and analysis of the waveform data non-trivial.

Accordingly, to develop a digital assessment for neuromuscular disorders, this example study was conducted to determine whether the biometric sensor device could measure facial muscle and eye movements. Some specific aims of the study were to: to determine how the biometric sensor device EMG/EOG/EEG signals may be processed to extract features; to determine biometric sensor device feature data quality, test re-test reliability, and statistical properties; to determine whether features derived from the biometric sensor device device can quantify various facial and ocular muscle activities; and to determine what features are important (e.g., are clinically relevant features 50) for activity level classification, in comparison to raw bio-signal data classification approaches.

In this study, 16 mock Performance Outcome Assessments (mock-PerfOs) were designed to assess facial and eye movements with the biometric sensor device on N=10 control volunteer participants. A fit-for-purpose feature engineering pipeline is implemented, where features from the EMG, EOG, and EEG waveforms are derived, feature relationships are evaluated in comparison to each other, and qualitatively assessment of how features classify different mock-PerfO activities is conducted. The steps taking in this example study are supportive of the usability and analytical validation steps of the framework for the development of digital assessments. Taken together, the results from this example study highlight the utility of the biometric sensor device as a potential measurement tool in a clinical trial setting for evaluating facial and eye movement tasks, and enable further clinical development with this and similar devices. The utility of the biometric sensor device is determined by the identification of sufficient clinically relevant features 50, as discussed herein.

To evaluate how well the biometric sensor device can classify facial muscle and eye movement tasks (e.g., to distinguish between the same), the study with N=10 participants who performed 16 facial muscle task movements (mock-PerfOs) four times in the morning and four times in the evening was conducted. Table 1 shows the demographic characteristics of the study participants.

TABLE 1

| Characteristic | Mean (SD) |
|---|---|
| Sex | |
| Male (N, %) | 5, 50% |
| Female (N, %) | 5, 50% |
| Age (years) | 32.03 (11.66) |
| Height (inches) | 71.33 (14.83) |
| Weight (pounds) | 154.8 (21.83) |
| BMI (kg/m²) | 22.91 (5.33) |

The biometric sensor device's raw bio-signal data was processed into 161 summary features, most of which describe the EMG, EOG, and EEG waveforms. As shown in FIG. 1A, the biometric sensor device (signal capture device 10) provided raw signals to a signal manipulation module 20. Signal manipulation module 20 generated distinct signals 30 (e.g., the EMG, EOG, and EEG waveforms) from the raw signals. 161 summary features were generate based on properties of the distinct signals 30.

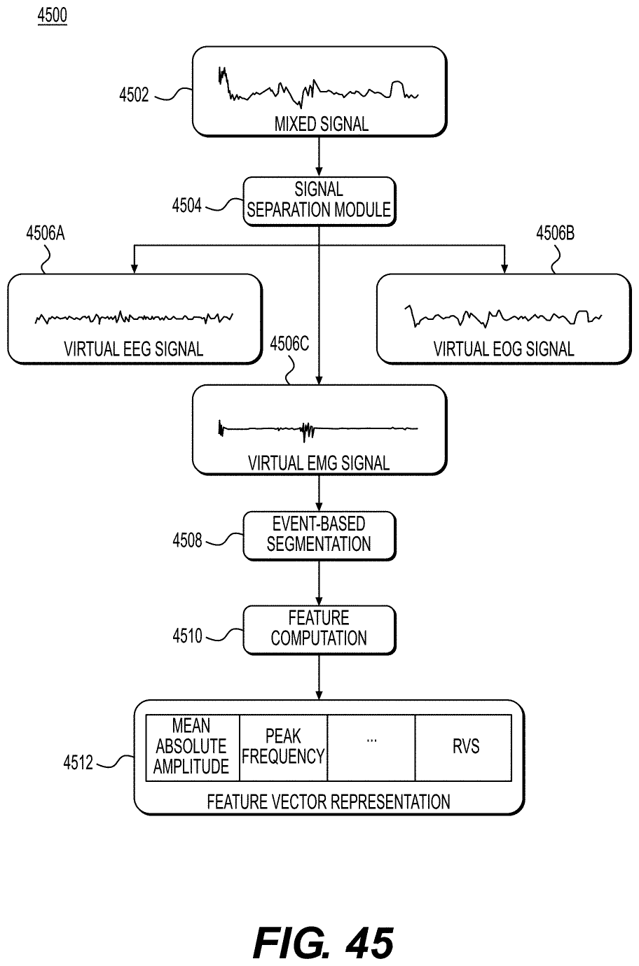

The process to summarize the raw biometric sensor device bio-signal data into features is described in detail herein. Briefly, features were computed from EMG, EOG, and EEG waveform components that were separated from raw, mixed-waveform bio-signals through specialized signal combination and filtering mechanisms (e.g., by signal manipulation module 20). A high-level overview of the feature engineering process 4500 is also summarized in FIG. 45.

As shown in FIG. 45, a mixed signal waveform 4502 is received from the biometric sensor device. Signal separation module 4504 may extract distinct virtual EEG signal 4506A, virtual EOG signal 4506B, and virtual EMG signal 4506C from the mixed signal waveform 4502. An event-based segmentation algorithm may be applied at event-based segmentation algorithm 4508 and feature computation at 4510 may result in feature extraction via feature vector representations 4512. As shown, signal separation module 4504 is applied to the mixed signal derived from the biometric sensor device, to separate the EEG, EMG, and EOG waves into their component parts. These signals are then subject to an event-based segmentation algorithm 4508, and features extracted.

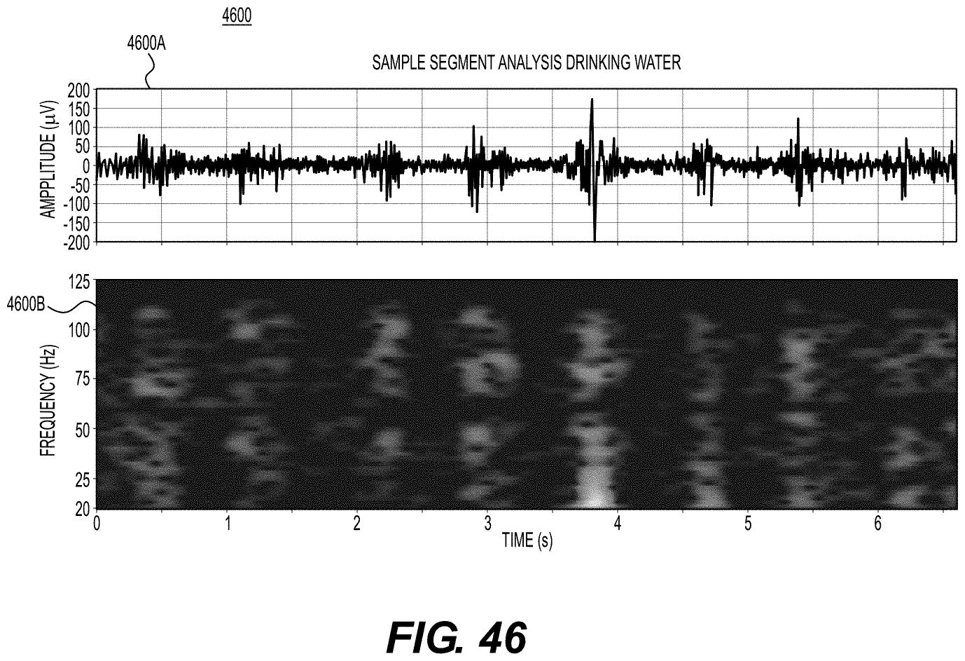

As shown in FIG. 46, features may be representative of both the frequency and time domain of the biometric device signal, via amplitude in time 4600A and frequency 4600B, collected for a subject drinking water. FIG. 46 shows time and frequency representations of EMG activity resulting from a participant drinking water. Plot 4600 shows approximately 6.5 s of EMG data in both the time 4600A and frequency 4600B domains.

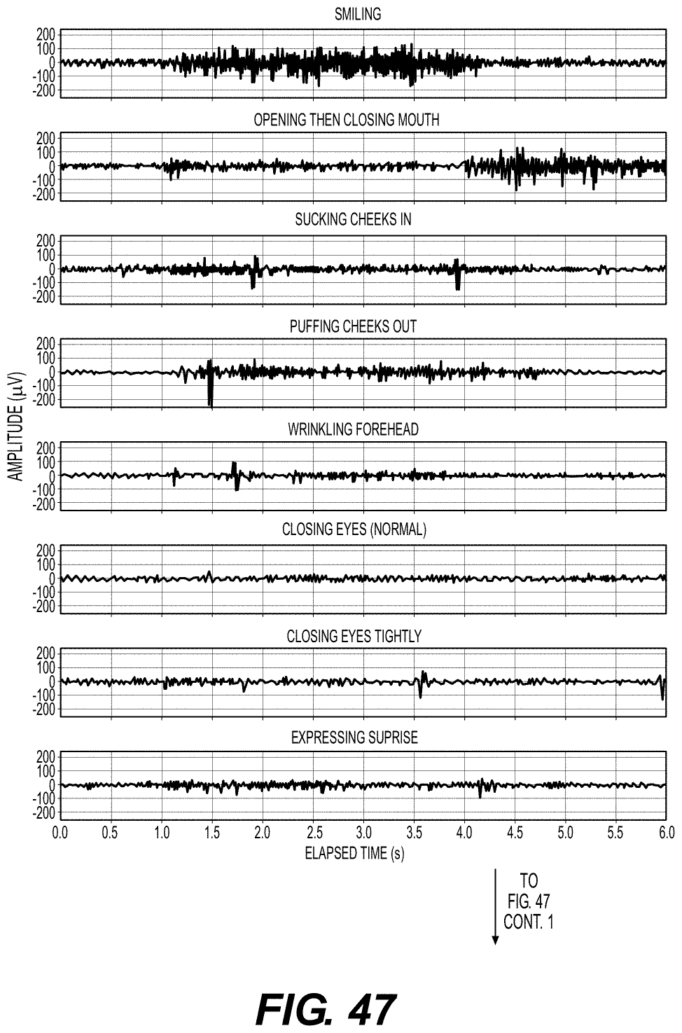

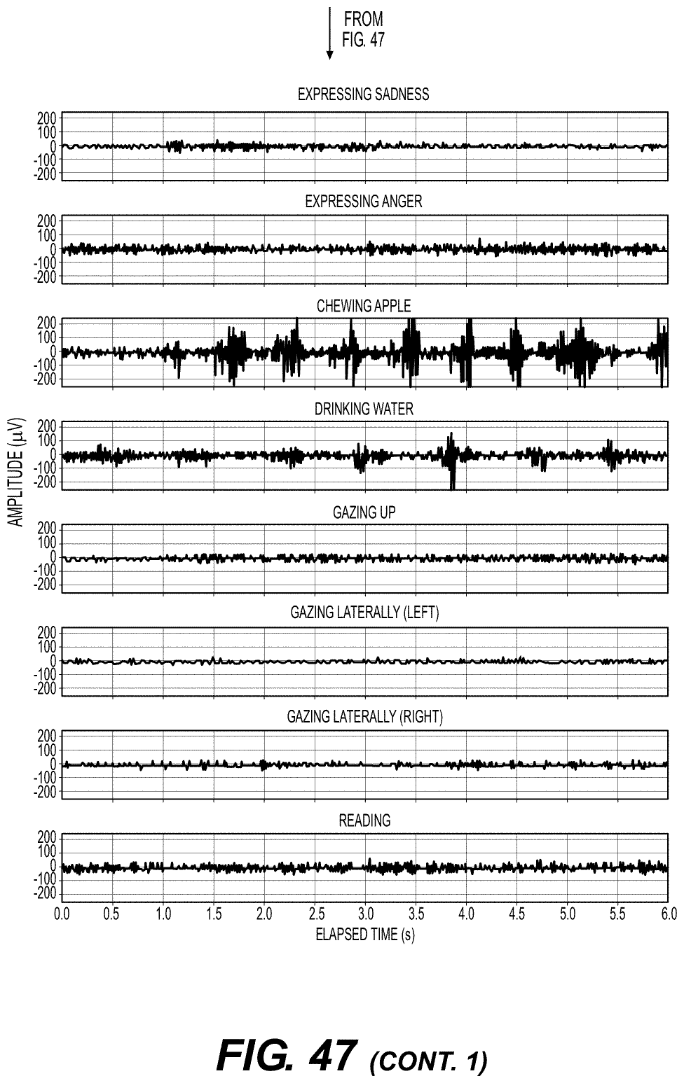

Representative mixed signal waveform 4502 are collected for each of the 16 mock-PerfO activities. For example, FIG. 47 shows qualitative differences observed from representative signals 4700 from each of the 16 mock-PerfO. As shown, each activity has a qualitatively different waveform. The representative signals 4700 show EMG activity visualized in the time domain over 16 activities.

As an example, the plurality of features extracted from the representative signals 4700, from each of the 16 mock-PerfO, may be used to generate Z score heat maps as further discussed herein and also shown in FIG. 8 and FIG. 9.

Table 2 shows the list of features extracted in this example, the features described in categories described. Amplitude features, zero crossing rate, standard deviation, variance, root mean square, kurtosis, frequency, bandpower, skew2 as well as other standard waveform features were processed from biometric sensor data. Features were selected according to standard feature processing pipelines. Amplitude features describe the amplitude or maximum distance from baseline of each wave in the relevant component space. Bandpower features describe the average power of a wave in a specific frequency range (where there are multiple frequency ranges specific to each signal type for the biometric sensor device). Other named features mathematically describe the shape, variance, or complexity of the EMG, EOG, or EEG waveforms.

TABLE 2

| Feature Name | Units | Group | Domain |
|---|---|---|---|
| EOG 0.5-4 Hz Bandpower Channel 1 | decibel | Bandpower | Frequency |
| EOG 4-10 Hz Bandpower Channel 1 | decibel | Bandpower | Frequency |
| EOG 0.5-4 Hz Bandpower Channel 2 | decibel | Bandpower | Frequency |
| EOG 4-10 Hz Bandpower Channel 2 | decibel | Bandpower | Frequency |
| EEG l-4 Hz Bandpower Channel 1 | decibel | Bandpower | Frequency |
| EEG 4-7 Hz Bandpower Channel 1 | decibel | Bandpower | Frequency |
| EEG 7-12 Hz Bandpower Channel 1 | decibel | Bandpower | Frequency |
| EEG 12-30 Hz Bandpower Channel 1 | decibel | Bandpower | Frequency |
| EEG l-4 Hz Bandpower Channel 2 | decibel | Bandpower | Frequency |
| EEG 4-7 Hz Bandpower Channel 2 | decibel | Bandpower | Frequency |
| EEG 7-12 Hz Bandpower Channel 2 | decibel | Bandpower | Frequency |
| EEG 12-30 Hz Bandpower Channel 2 | decibel | Bandpower | Frequency |
| EMG 10-33 Hz Bandpower Channel 1 | decibel | Bandpower | Frequency |
| EMG 33-56 Hz Bandpower Channel 1 | decibel | Bandpower | Frequency |
| EMG 56-79 Hz Bandpower Channel 1 | decibel | Bandpower | Frequency |
| EMG 79-102 Hz Bandpower Channel 1 | decibel | Bandpower | Frequency |
| EMG 102-125 Hz Bandpower Channel 1 | decibel | Bandpower | Frequency |
| EMG 10-33 Hz Bandpower Channel 2 | decibel | Bandpower | Frequency |
| EMG 33-56 Hz Bandpower Channel 2 | decibel | Bandpower | Frequency |
| EMG 56-79 Hz Bandpower Channel 2 | decibel | Bandpower | Frequency |
| EMG 79-102 Hz Bandpower Channel 2 | decibel | Bandpower | Frequency |
| EMG 102-125 Hz Bandpower Channel 2 | decibel | Bandpower | Frequency |
| Spectral Entropy Channel 1 | unitless | Complexity | Frequency |
| Spectral Entropy Channel 2 | unitless | Complexity | Frequency |
| EMG Peak Frequency Contractions Channel 1 | Count | Frequency | Frequency |
| EMG Peak Frequency Contractions Channel 2 | Count | Frequency | Frequency |
| Peak Frequency 1 Channel 1 | Hertz | Frequency | Frequency |
| Peak Frequency 2 Channel 1 | Hertz | Frequency | Frequency |
| Peak Frequency 3 Channel 1 | Hertz | Frequency | Frequency |
| Peak Frequency 4 Channel 1 | Hertz | Frequency | Frequency |
| Peak Frequency 5 Channel 1 | Hertz | Frequency | Frequency |
| Peak Frequency 1 Channel 2 | Hertz | Frequency | Frequency |
| Peak Frequency 2 Channel 2 | Hertz | Frequency | Frequency |
| Peak Frequency 3 Channel 2 | Hertz | Frequency | Frequency |
| Peak Frequency 4 Channel 2 | Hertz | Frequency | Frequency |
| Peak Frequency 5 Channel 2 | Hertz | Frequency | Frequency |
| Mean Absolute Amplitude Channel 1 | microvolts | Amplitude | Time |
| Mean Absolute Amplitude Channel 2 | microvolts | Amplitude | Time |
| Max Absolute Amplitude Channel 1 | microvolts | Amplitude | Time |
| Max Absolute Amplitude Channel 2 | microvolts | Amplitude | Time |
| 25th Percentile Absolute Amplitude Channel 1 | microvolts | Amplitude | Time |
| 50th Percentile Absolute Amplitude Channel 1 | microvolts | Amplitude | Time |
| 75th Percentile Absolute Amplitude Channel 1 | microvolts | Amplitude | Time |
| 90th Percentile Absolute Amplitude Channel 1 | microvolts | Amplitude | Time |
| 25th Percentile Absolute Amplitude Channel 2 | microvolts | Amplitude | Time |
| 50th Percentile Absolute Amplitude Channel 2 | microvolts | Amplitude | Time |
| 75th Percentile Absolute Amplitude Channel 2 | microvolts | Amplitude | Time |
| 90th Percentile Absolute Amplitude Channel 2 | microvolts | Amplitude | Time |
| Standard Deviation Channel 1 | microvolts | Amplitude | Time |
| Standard Deviation Channel 2 | microvolts | Amplitude | Time |
| Absolute Amplitude Standard Deviation Channel 1 | microvolts | Amplitude | Time |
| Absolute Amplitude Standard Deviation Channel 2 | microvolts | Amplitude | Time |
| Root Mean Square Channel 1 | microvolts | Amplitude | Time |
| Root Mean Square Channel 2 | microvolts | Amplitude | Time |
| EOG Initial Deflection Max Amplitude Channel 1 | microvolts | Amplitude | Time |
| EOG Initial Deflection Max Amplitude Channel 2 | microvolts | Amplitude | Time |
| EOG Initial Deflection Polarity Channel 1 | microvolts | Amplitude | Time |
| EOG Initial Deflection Polarity Channel 2 | microvolts | Amplitude | Time |
| Detrended Fluctuation Hurst Parameter Channel 1 | unitless | Complexity | Time |
| Detrended Fluctuation Hurst Parameter Channel 2 | unitless | Complexity | Time |
| Petrosian Fractal Dimension Channel 1 | unitless | Complexity | Time |
| Petrosian Fractal Dimension Channel 2 | unitless | Complexity | Time |
| Approximate Entropy Channel 1 | unitless | Complexity | Time |
| Approximate Entropy Channel 2 | unitless | Complexity | Time |
| Zero Crossing Rate Channel 1 | Occurrences/Second | Frequency | Time |
| Zero Crossing Rate Channel 2 | Occurrences/Second | Frequency | Time |
| Amplitude Kurtosis Channel 1 | unitless | Kurtosis | Time |
| Amplitude Kurtosis Channel 2 | unitless | Kurtosis | Time |
| Amplitude Skew Channel 1 | unitless | Skew | Time |
| Amplitude Skew Channel 2 | unitless | Skew | Time |
| Perceptible Onset Time | seconds | Time | Time |
| Amplitude Variance Channel 1 | Microvolts squared | Variance | Time |
| Amplitude Variance Channel 2 | Microvolts squared | Variance | Time |

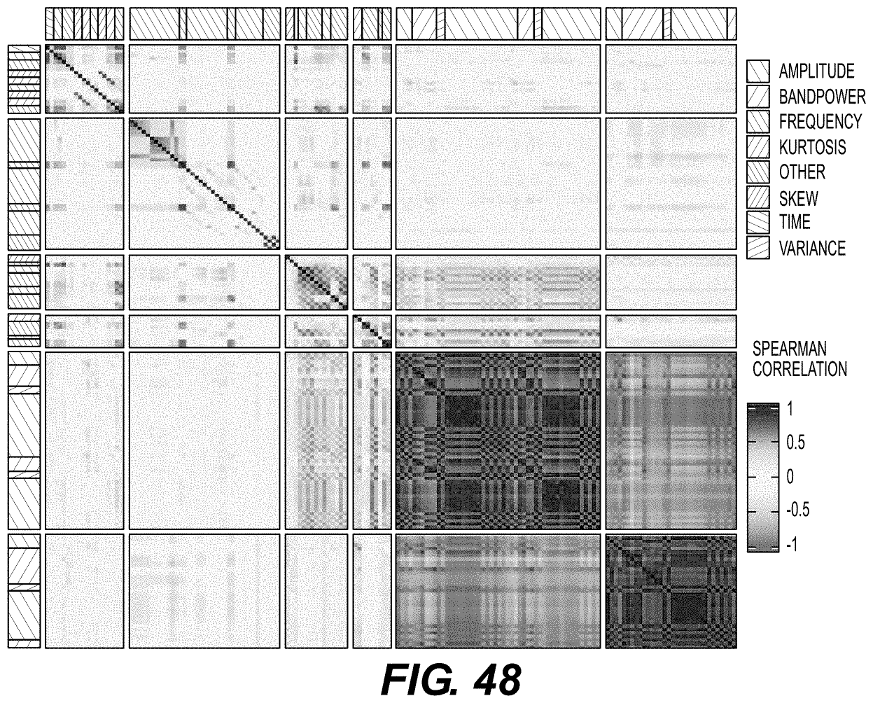

The biometric sensor device based features outlined in Table 2 above may measure unique aspects of facial and eye movement. The relationship between the parameters across all 16 mock-PerfO activities were analyzed by performing spearman correlations of all parameters against each other (e.g., in a manner described relative to FIG. 4C and FIG. 10). To determine the optimal number of parameter groups that describe the variation in the overall signal, k means clustering of the spearman correlations of all features is performed for all activities against each other, as shown in FIG. 48. Six unique clusters of parameters were determined (based on the k means clustering) from the spearman correlations. K-means clustering is a method of vector quantization to partition n observations (e.g., features based on spearman correlations) into k clusters in which each observation belongs to the cluster with the nearest mean, serving as a prototype of the cluster.

As discussed herein, spearmen correlation chart 4800 is used to identify relationships between features. Features that are highly correlated are likely measuring similar aspects of facial biology and/or other signals (e.g., electrical activity) collected by biometric sensor device (e.g., similar aspects of distinct signals 30). Spearmen correlation chart 4800 is used to identify the six clusters such that similar features within the same cluster may be omitted or otherwise reduced, to reduce duplication of analysis. For example, cluster based reduction may be applied to identify clinically relevant features 50. FIG. 47 shows the spearman correlation of all 161 Earable features against each other, represented as a heatmap. K=6 clusters from K means clustering (optimal number) are shown. All 16 mock-PerfOs are pooled for the correlation analysis shown in FIG. 47.

In this example, amplitude and bandpower parameters tended to cluster together in two of the six clusters, while other parameters like those from the frequency domain clustered separately.

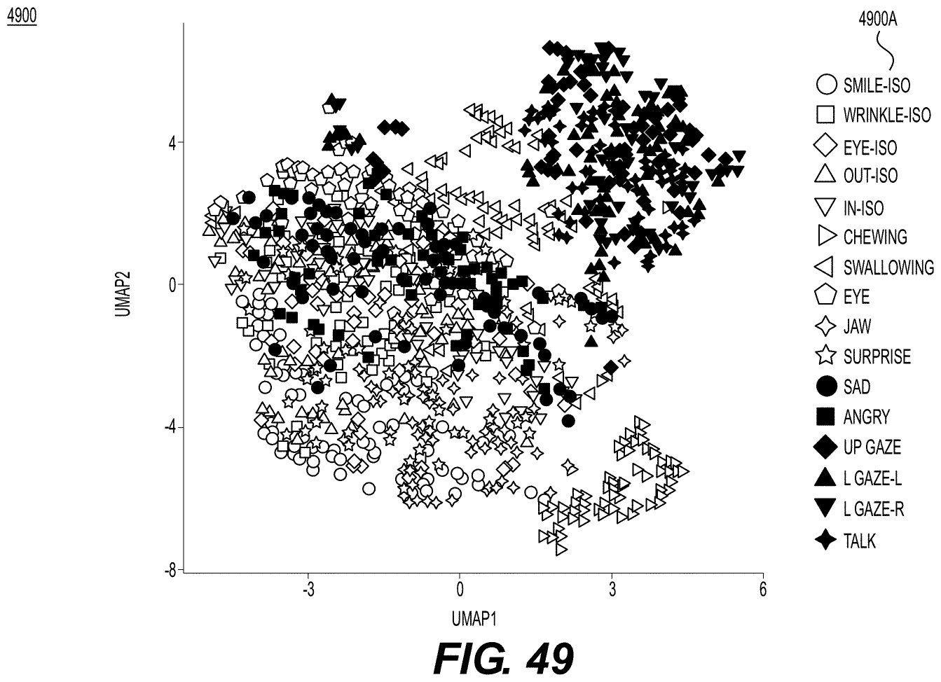

To investigate qualitative differences between the 16 mock-PerfO activities (across each participant and timepoint, and all 161 biometric sensor device parameters), UMAP dimensionality reduction was performed. Qualitative differences between the 16 mock-PerfO activities 4900A are shown in chart 4900 of FIG. 49. While there was overlap between some of the activities 4900A, activities like swallowing clearly separated out from the rest with this approach. UMAP dimensionality reduction shown in chart 4900 may be used to identify clusters of data and/or to segregate data by a dimension (e.g., by tasks). UMAP dimension reduction of all 161 biometric sensor device features is shown in FIG. 49. Each individual activity repeat is a point on chart 4900. The visual feature of a given point represents the activities performed during that activity.

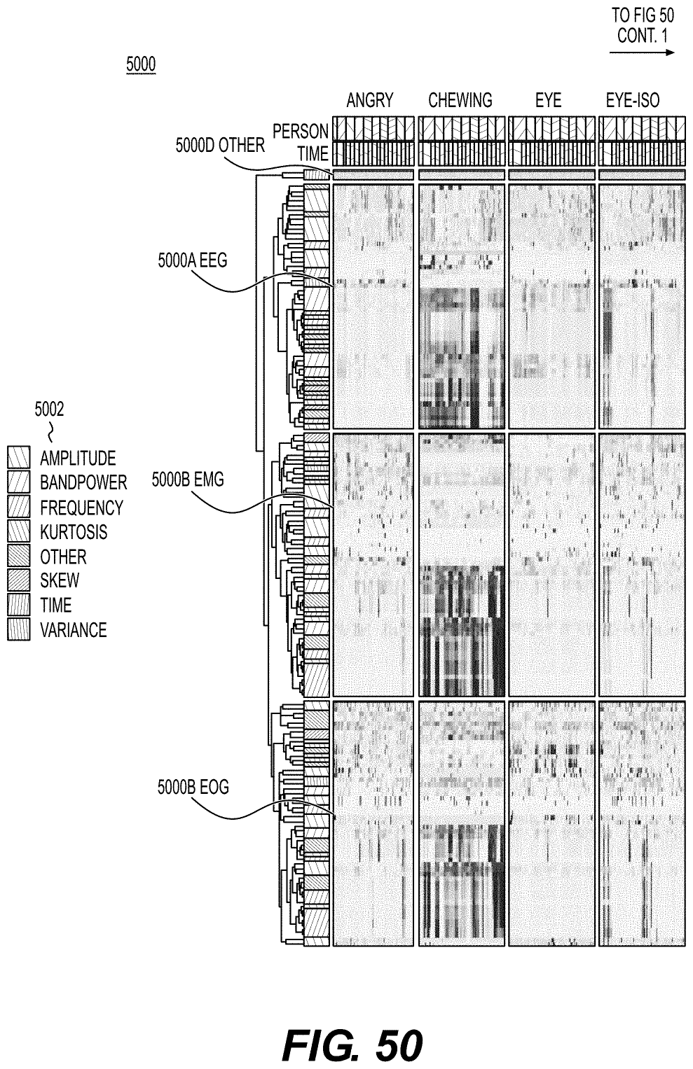

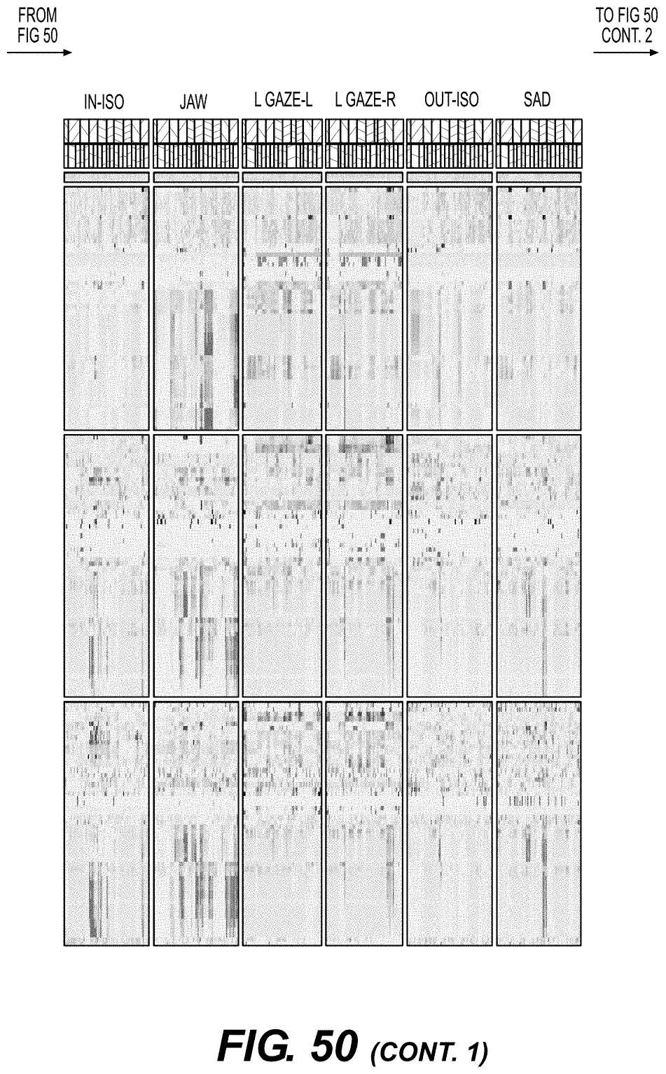

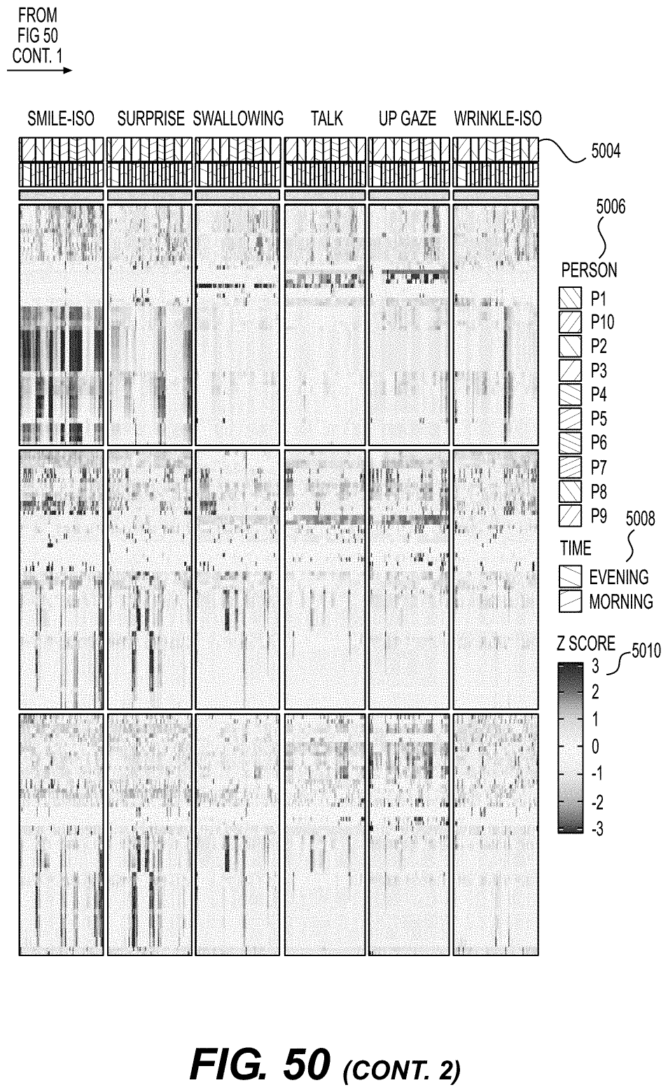

FIG. 50 includes chart 5000 that provides heat maps 5000A (EEG based features), 5000B (EMG based features), 5000C (EOG based features), and 5000D (other features) of feature z-score across data that demonstrate differences between the tasks 5004 for different classes 5002 of features (amplitude, bandpower, frequency, kurtosis, other, skew, time, variance). Data for each individual 5006 is collected for times 5008 and represented as a Z score based on legend 5010, which range in values from −3 to +3 and assigned a color. Chart 5000 may be generated and may include information in a manner similar to FIGS. 8, 9, and 24 discussed herein. Taken together, the results shown in chart 5000 demonstrate the utility of the biometric sensor device to generate parameters that may describe unique mock-PerfO activities.

FIG. 50 includes heat maps of all 161 biometric sensor device features (rows) for all activity repeats (columns). Columns are sorted first by the 16 activates in the study, and within each activity, by participant, and then time of day when the activity was performed, as discussed above.

To evaluate biometric sensor device feature test re-test reliability, linear mixed effects modeling with participants as random effects to evaluate feature properties is used. Table 3 shows a variance component analysis of the biometric sensor device features. First, for each of the 161 biometric sensor device features, ICC for participants to assess the variance associated with each person for each feature is determined, based on the flow of Table 4. As discussed herein, ICC is a measurement of how similar and, thus, reliable the same data from the same participant are for the same activity, and ranges from 0 to 1 (e.g., ICC less than 0.5 would be poor reliability, an ICC of 0.5-0.7 would be moderate reliability, and ICC greater than 0.7 may be interpreted as a reliable metric). Observed ICC values ranged from 0-0.92, and the average ICC value for all parameters across the 16 activities was 0.31. Second, CVs for each parameter within a participant across timepoints (morning and evening) is calculated in accordance with the techniques disclosed herein. The variance for each feature for each activity, associated with time of day activities were performed (morning or evening), individual participants themselves, and individual trial repeats, as well as the unexplained variance is computed. The ICC computation, the CV, and/or the variability are used to, for example, identify clinically relevant features 50 from extracted features 40, as shown in FIG. 1A. In this example, the results support that many biometric sensor device features reliably measuring intra-participant variation and provide a metric by which one may rank candidate features for further downstream analysis. Accordingly, such features may be designated clinically relevant features 50.

TABLE 3
Biometric sensor device Feature
Task 1

| Morning | | | | Evening | | | | |
|---|---|---|---|---|---|---|---|---|
| Participant 1 | | Participant 2 | | Participant 1 | | Participant 2 | | |
| Repeat | Repeat | Repeat | Repeat | Repeat | Repeat | Repeat | Repeat | |
| 1 | 2 | 1 | 2 | 1 | 2 | 1 | 2 | |
| | | | | | | | | Variance |
| Biometric sensor device Feature | | | | | | | | component |
| Task 2 | | | | | | | | Task |
| Morning | | | | Evening | | | | Time |
| Participant 1 | | Participant 2 | | Participant 1 | | Participant 2 | | Participant |
| Repeat | Repeat | Repeat | Repeat | Repeat | Repeat | Repeat | Repeat | Repeat |
| 1 | 2 | 1 | 2 | 1 | 2 | 1 | 2 | |

TABLE 4

Tab 1: ICC values for each of the 16 activities for all biometric
sensor device parameters.
Tab 2: % CV values for each of the 16 activities for all biometric
sensor device parameters.
Tab 3: Number of tasks analyzed (n), the minimum value (min),
maximum value (max), median value (median), mean value (mean),
standard deviation of the mean (sd), and standard error of the
mean (se).
Tab 4: Variance components for each biometric sensor device
feature for each of the 16 activities as shown in Table 3.

It is determined that the tested biometric sensor device can accurately classify some facial muscle movement activities. To investigate whether the biometric sensor device data is able to classify a given one of the sixteen mock-PerfO activities, a Random Forest classification model discussed herein is constructed to detect each activity from the other fifteen activities (1-against-all classification) (e.g., as discussed in reference to FIGS. 29, 43, and 44). Activity detection F1 scores are used as the primary metric for evaluating model performance, as further discussed herein.

Following developmental evaluation on the testing dataset for all 161 features, a second model is built for activity-level classification. This second model uses an optimized set of biometric sensor device features with the goal of eliminating noisy features that would not contribute to overall classification performance. For determination of the optimized set of biometric sensor device features, feature reduction with the Boruta package is performed. Such feature reduction is discussed in reference to FIGS. 29, 43, and 44, herein. For example, all 161 to FIGS. 29, 43, and 44 features are copied (e.g., designated as shadow features), and their class labels are randomly shuffled. Each shadow feature is compared to the real values for 1,000 iterations of classification, and only features that perform better than a given threshold (e.g., 50%) are designated as confirmed. This analysis indicated an confirmed set of 101 to FIGS. 29, 43, and 44 features that were be used for a second classification model.

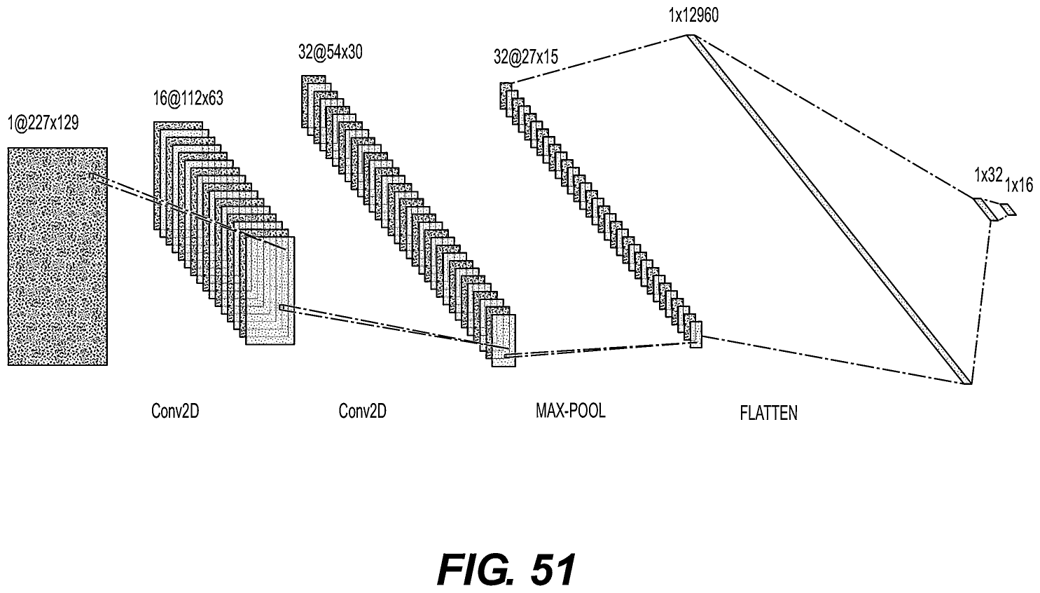

To evaluate how well biometric sensor device features perform relative to using low level representations of the biometric sensor device waveform data, CNN models are built with the biometric sensor device raw bio-signal data to classify the 16 mock-PerfO activities, as shown in FIG. 51. FIG. 51 shows a model 5100 that implements an architecture diagram of the final CNN implemented for activity classification. A single channel spectrogram computed from the segmented waveform is input to the model at classification time. A probability distribution over each of the 16 activities is output. The activity associated with the highest output likelihood estimate is inferred. In this modeling, fixed size spectrograms that quantify how the power at a given frequency changes as a function of time is computed from the mock-PerfO signal segments and used as model input.

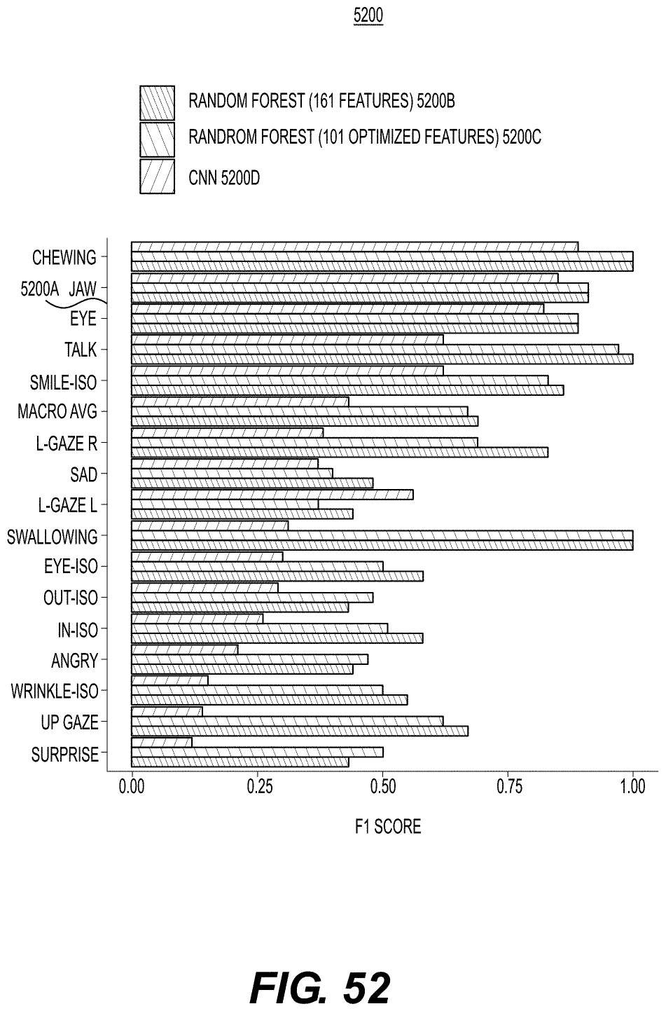

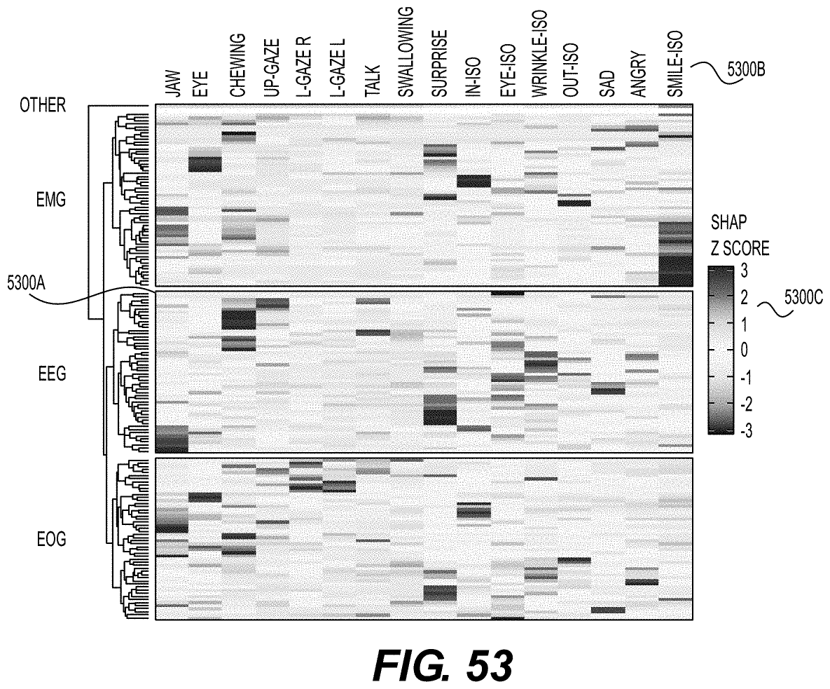

FIG. 52 shows activity chart 5200 for activities 5200A with level classification F1 scores for all biometric sensor device features (161 features) 5200B, Boruta selected biometric sensor device features (101 features) 5200C, and using raw waveform data (CNN) 5200C. F1 scores range from 0 to 1, with 1 indicating perfect classification. Boruta selected biometric sensor device features (101 features) 5200C may be clinically relevant features 50 extracted from the biometric sensor device features (161 features) 5200B (e.g., as shown in FIGS. 29, 43, and 44). Raw waveform data (CNN) 5200C may be generated using the model 5100 of FIG. 51. FIG. 53 includes heat map 5300 that shows feature attribution analysis using SHapley Additive exPlanations (SHAP) values for each feature (row) 5300A for each activity (columns) 5300B determined on the model from the full set of 161 features. SHAP values are z scored across all activities, as indicated in legend 5300C, which range in values from −3 to +3 and assigned a color.

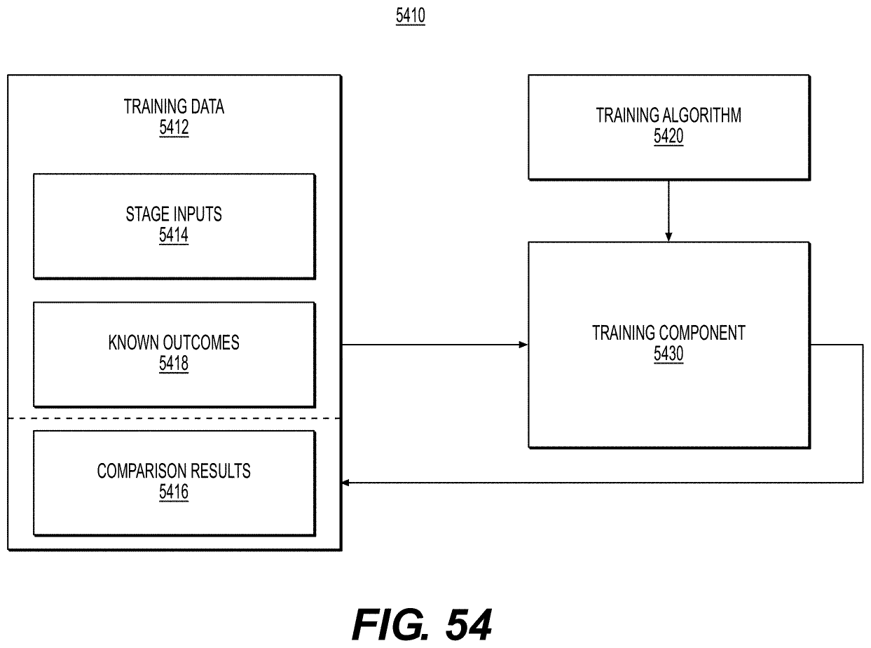

As shown in FIG. 52, the F1 scores for classification accuracy of the full set of 161 features 5200B, the optimized set of 101 features 5200C, as well as the predictions from the CNN 5200D are compared. To determine the underlying features that are most important for the full RF model with 161 features (e.g., the clinically relevant features 50), feature attribution analysis with SHAP is applied in FIG. 54. For a specific activity prediction, the SHAP value of a biometric sensor device feature is computed as the change in the expected value of the model output when this feature is observed, compared to when it is missing for the test set predictions. The effect of each feature as it is added to the model is summed and averaged across all 161 biometric sensor device features used. In FIG. 53, the features are represented as the Mean average SHAP value and are shown in a heat map (log 10) 5300. The percent contribution for each activity of each waveform group of features is determined and shown in Table 5. The normalized sum of absolute SHAP values for each activity is compared against the sum within the EMG, EEG, and EOG features, and normalized by the number of features in that group to calculate the percent contribution of each waveform to the classification accuracy.

TABLE 5

| | Normalized Sum | EMG % | EEG % | EOG % |
|---|---|---|---|---|
| | of Absolute | Contri- | Contri- | Contri- |
| Activity | SHAP Values | bution | bution | bution |
| L Gaze-R | 0.517996133 | 48.5 | 21.5 | 30.0 |
| L Gaze-L | 0.49281577 | 48.1 | 22.9 | 29.0 |
| Up Gaze | 0.526086224 | 48.2 | 27.9 | 23.9 |
| Chewing | 0.669707634 | 49.8 | 27.3 | 22.9 |
| Eye-Iso | 0.50169248 | 58.1 | 22.3 | 19.5 |
| Out-Iso | 0.478709587 | 58.3 | 22.6 | 19.1 |
| Eye | 0.671975561 | 62.9 | 18.1 | 18.9 |
| In-Iso | 0.553403349 | 60.4 | 20.8 | 18.8 |
| Jaw | 0.759305572 | 62.4 | 20.5 | 17.1 |
| Surprise | 0.502448985 | 57.5 | 25.7 | 16.8 |
| Wrinkle-Iso | 0.528007475 | 56.7 | 26.7 | 16.6 |
| Talk | 0.577886127 | 61.4 | 22.4 | 16.2 |
| Angry | 0.485578854 | 66.6 | 18.0 | 15.4 |
| Sad | 0.468684661 | 67.4 | 17.4 | 15.3 |
| Swallowing | 0.784064192 | 68.9 | 17.9 | 13.2 |
| Smile-Iso | 0.558172499 | 75.6 | 16.0 | 8.4 |

Table 5 includes the 16 mock-PerfO activities and indicates how EMG, EEG, and EOG feature groups contribute to classification accuracy. Table 5 shows the normalized sum of the Absolute SHAP values from the RF model, as well as the relative EMG, EEG, and EOG percent contributions to classification importance. Feature importance is normalized based on the total number of features in each EMG, EEG, or EOG group, compared to the total number of features in all three categories. Features not associated with any waveform are excluded from this analysis.

As disclosed herein, a total of 10 healthy volunteers (5 male and 5 female) contributed to this example study. All participants completed two 45-minute sessions. During each session, each participant was asked to complete a series of tasks listed in Table 6 below. These tasks were chosen to represent tasks MG patients may have difficulty completing. Participants were asked to take a one-minute break between each task.

TABLE 6

| | | | | Total duration | |
|---|---|---|---|---|---|
| | Duration for | | | (including task | |
| Task | each task | | Repeats | preparation) | |
| ICF, introduction and Q&A | 5 | minutes | N/A | 5 | minutes |
| Earable device setup | 5 | minutes | N/A | 5 | minutes |
| Smile broadly and show | 15 | seconds | 4 | 1 | minute |
| teeth as hard as possible | | | | | |
| Wrinkle forehead as | 15 | seconds | 4 | 1 | minute |
| tightly as possible | | | | | |
| Close eyes normally | 5 | seconds | 4 | 20 | seconds |
| Close eyes as tightly as | 15 | seconds | 4 | 1 | minute |
| possible | | | | | |
| Puff out cheeks as much | 15 | seconds | 4 | 1 | minute |
| as possible | | | | | |
| Suck in cheeks as much | 15 | seconds | 4 | 1 | minute |
| as possible | | | | | |
| Chewing | 30 | seconds | 4 | 2 | minutes |
| Swallowing | 10 | seconds | 4 | 40 | seconds |
| Upwards gaze | 45 | seconds | 4 | 3 | minutes |
| Lateral gaze-left | 45 | seconds | 4 | 3 | minutes |
| Lateral gaze-right | 45 | seconds | 4 | 3 | minutes |
| Talking | 30 | seconds | 4 | 2 | minutes |
| Open and close the jaw | 15 | seconds | 4 | 1 | minute |
| as much as possible | | | | | |
| Facial expression-surprise | 15 | seconds | 4 | 1 | minute |
| (for explorative purposes | | | | | |
| only) | | | | | |
| Facial expression-sad | 15 | seconds | 4 | 1 | minute |
| (for explorative purposes | | | | | |
| only) | | | | | |
| Facial expression-angry | 15 | seconds | 4 | 1 | minute |
| (for explorative purposes | | | | | |
| only) | | | | | |

| | | |
|---|---|---|
| Total Time | 33 | minutes |

Each study participant engaged in two study sessions, one in the morning and one at night. Testing sessions were conducted one-on-one by a study moderator. In the morning session, the study moderator reviewed the informed consent form (ICF) with the participant, ensured that he/she understood the form and agreed to participate. The participants had time to ask questions before signing the ICF.

The study moderator read a study script, which provided a study overview and description of various study activities. The study moderator then collected participants' baseline (background) information.

The study moderator then had participants perform the following at each study session:

Smile broadly and show teeth as hard as possible

1-minute break

Wrinkle forehead as tightly as possible

1-minute break

Close eyes as tightly as possible

1-minute break

Put out cheeks as much as possible

1-minute break

Suck in cheeks as much as possible

1-minute break

Chewing for 30 seconds

1-minute break

Swallowing

1-minute break

Close eye normally for 5 seconds

1-minute break

Talking 30 seconds

1-minute break

Upward gaze for 45 seconds

1-minute break

Lateral gaze left for 45 seconds

1-minute break

Lateral gaze left for 45 seconds

1-minute break

Open and close jaw as much as possible

1-minute break

Facial expression—surprise

1-minute break

Facial expression—sad

1-minute break

Facial expression—angry

Label annotations disclosed herein correspond to the following tasks outlined in Table 7.

TABLE 7

| Task | Label/Annotation |
|---|---|
| Smile broadly and show teeth as hard as possible | Smile-Iso |
| Wrinkle forehead as tightly as possible | Wrinkle-Iso |
| Close eyes as tightly as possible | Eye-Iso |
| Puff out cheeks as much as possible | Out-Iso |
| Suck in cheeks as much as possible | In-Iso |
| Chewing | Chewing |
| Swallowing | Swallowing |
| Close eyes normally | Eye |
| Open and close jaw as much as possible | Jaw |
| Upwards gaze | Up Gaze |
| Lateral gaze-left | L Gaze-L |
| Lateral gaze-right | L Gaze-R |
| Talking | Talk |
| Facial expression-surprise | Surprise |
| Facial expression-sad | Sad |
| Facial expression-angry | Angry |

Raw biometric sensor device data was continuously collected during each activity of this example study. To guarantee reliable ground truth data annotations, data from each activity was manually labeled by an expert technician. For each activity, the onset and offset endpoints of each performed activity were annotated accordingly. A time-synchronized video recording of the participant was utilized as a reference source in this annotation procedure. Using these activity annotations, signals were then segmented according to noted onset and offset timestamps. It will be understood that raw data collection, in accordance the techniques disclosed herein, may be conducted automatically by using sensors that transmit the raw data to one or more receivers or controllers (e.g., as shown in FIGS. 1-3).

After completion of an activity, the resulting signals from each channel were scaled to counteract the effects of amplification performed in device hardware for the purpose of noise suppression and filtered offline using a second-order infinite impulse response (IIR) notch filter to remove 60 Hz power line noise. Each signal included a mixture of EEG, EMG, and EOG data (e.g., mixed signal waveform 4502). A signal separation algorithm was applied (e.g., by signal separation module 4504) to better isolate each component, yielding a total of six channels (two each for EEG, EMG, EEG) in this example.

Following signal scaling, filtering, and separation, the signals of each of the six separated channels were segmented based on the presence or absence of facial movement activity, as shown in FIG. 45. A comprehensive approach to feature extraction was taken for further downstream analysis (e.g., 4510 of FIG. 45). General features for each waveform, apart from a subset of features specific to EMG, EOG, or EEG activity, were summarized. Features that would clearly identify mock-PerfO activities performed within the data collection process but would not generalize to performance of the activity outside of laboratory contexts were omitted (for instance, duration of an activity that each participant was instructed to perform for a specified period).

Event-based segmentation algorithm 4508 and feature computation 4510 was conducted. Statistical measures from each separated signal segment were computed to summarize signal behavior in the time-domain (e.g., see FIG. 46). Such measures allow depiction of information such as time-varying amplitude behavior, amplitude distributions, and signal trends observable in their raw forms. As the frequency and time-frequency domains also include vast amounts of information in bio-signal data, digital signal processing (DSP) analyses were performed to decompose each separated signal segment into frequency components and evaluate patterns in this alternative representation, as shown in FIG. 45. Features relevant to theoretical EMG, EOG, and EEG behavior during specific mock-PerfO activities were computed to better represent such activities in the summary feature vectors.

As discussed herein, the steps outlined above (as also shown in FIG. 45) yielded 161-dimension feature vector representations for each mock-PerfO activity performed, as outlined in Table 2. These features correspond to the extracted features 40 of FIG. 1A. To remove features potentially irrelevant to activity-based classification for a given clinical outcome being studied, feature reduction using the Boruta algorithm was implemented. As shown in FIG. 52 the total number of 161 features was trimmed, yielding a lower dimensionality feature vector representations of each mock-PerfO activity. As shown, 60 features that were estimated as “unimportant” were removed from each feature vector, resulting in 101-dimension feature vectors. A Python implementation of the Boruta package (BorutaPy, version 0.3) was used to preform feature reduction.

Correlation of biometric sensor device parameters and differences in parameters between activities is observed. Spearman correlations between all parameters and all activities were computed, as shown in FIG. 48. A silhouette technique was used to determine the optimal number of clusters with the factoextra package in R with function fviz_nbclust with 100 bootstrapped samples. For each of the 16 activities, for all the 161 computed b parameters, we report the number of tasks analyzed (n), the minimum value (min), maximum value (max), median value (median), mean value (mean), standard deviation of the mean (sd), and standard error of the mean (se), as outlined in Table 4.

Relationships between biometric sensor device features and activity or demographic information were determined. For data from the example study, the ICC for participants as the group was computed using linear mixed-effects modeling with the lmer package in R, with the following formula: ˜(1|participant). ICC was computed separately for each of the 16 activities for each of the 161 biometric sensor device features in accordance with Table 4. Coefficients of variation were also computed comparing within each activity in accordance with Table 4.

Within and between trial variability due to repeated measures, time of day, and participants was computed. The variance not explained by these three factors was also computed, in accordance with Table 4. A nested linear mixed effects model to derive the variation explained by each component: ˜1+(1|time)+(1|participant)+(1|repeat/time) was used, where the time component indicates time of day (morning or evening), the participant component indicates the subject, and the repeat component indicates the repeat of the same activity nested within the same time.

As shown in FIGS. 25, 26, 27, 40, 41, 42, and 49, dimensionality reduction of biometric device features was performed in Python with umap-learn or one or more applicable dimensionality reduction techniques. The reduction was performed with an effective minimum distance between embedded points of one, and default parameters. For example, as shown in FIG. 49, UMAP coordinates were plotted with ggplot2 in R. As shown in FIG. 50, heat maps of biometric sensor device features are displayed with individual activities as columns and biometric sensor device features as rows. Heat maps (e.g., FIGS. 8-12, 22, 24, 31-36, 38, 39, 48, 50, and 53) of biometric sensor device data display z-scored feature rows, computed across all activities. Heat maps were constructed with the ComplexHeatmap package in R.

Study activities and participant-level predictions were quantified. To determine how biometric sensor device features could be used to classify each of the 16 activities multi-class classification models using the Python sklearn module were implemented. A random forest classifier (e.g., using the sklearn RandomForestClassifier class) with 500 decision trees for model building was implemented. In each classification setting model training and validation was performed using 80% of the dataset while the remaining 20% of the dataset with withheld for testing. Data samples were assigned to one of the two subsets at random to reduced bias in evaluation results. As shown in FIG. 52, the F1 score was calculated to evaluate model performances on the test set. The F1 score is the harmonic mean of precision and recall and elucidates the number of predictions that were accurate by the model, balancing both false negatives and false positives.

CNN models of activity level prediction were determined. Deep Learning models have been used to achieve high performance in many tasks relevant to classification of bio-signal data. Among the many popular Deep Learning architectures leveraged in such tasks, CNNs are widely used for their ability to learn patterns in structured, multidimensional data (e.g., time-frequency signal representations). In applying such methodologies to the task of mock-PerfO activity-level classification, 16-class CNN classification models were developed and analyzed. These CNN models were constructed to map 2-dimensional spectrogram representations of the mock-PerfO activity signal segments to a probability distribution over the 16 classes.

As Deep Learning models often require large datasets to learn generalizable functions, data augmentation was employed in effort to maximize the diversity in the training set. Each time a signal segment is read into the training data set, multiple random croppings of this segment are also added to the training set. To an extent, this allowed an increase in the size of the training dataset without collecting additional samples, helping to counter overfitting. To maintain constant length input signals among the mock-PerfO activities that varied in duration, activity segments shorter in duration than the fixed input data duration (e.g., 30 seconds) were repeated after shifting the segment according to the randomized cropping scheme, while segments longer in duration were truncated to the fixed input data duration via randomized cropping. Data augmentation was not performed for the testing set as it would bias the resulting model performance estimate. Additional techniques applied to reduce model variance included the use of L2 kernel regularization in the convolutional and fully connected model layers and the inclusion of Dropout layers throughout the network. Following development and evaluation on training and validation datasets, a shallow CNN, as shown in FIG. 51, was trained, and employed for testing purposes.

Data from this example study suggests that the tested biometric sensor device, as well as similar wearable devices, may be used for objective quantitation of cranial and eye muscle movements. The techniques disclosed herein (e.g., to identify clinically relevant features 50) may be used to identify the capabilities and boundaries of given devices, based on clinical outcomes. The techniques disclosed herein may be used to test the utility of a wearable device in disease populations, more accurately measure disease progression within participants, test how wearable device features or data relate to existing PROs, and/or more accurately measure treatment effects within disease populations. The use of the biometric sensor device in longitudinal studies where disease progression may be measured, for example ongoing natural history studies, may help elucidate which features are most important for quantifying disease effects. The exploratory use of these devices in clinical trials as part of a wearable clinical development strategy may enable more sensitive detection of treatment responses within disease populations. These clinical validation steps may additionally support a strategy to use devices like tested biometric sensor device for passive monitoring purposes. Such monitoring may be implemented by obtaining signals from signal capture device 10, identifying clinically relevant features 50 based on data collected by signal capture device 10, and/or using the clinically relevant features 50 to provide a clinical outcome on an ongoing (e.g., continuous) basis (e.g., identification of a disease or disorder and/or a treatment plan based on the same).

One or more implementations disclosed herein include a machine learning model. A machine learning model disclosed herein may be trained using the data flow 5410 of FIG. 54. As shown in FIG. 54, training data 5412 may include one or more of stage inputs 5414 and known outcomes 5418 related to a machine learning model to be trained. The stage inputs 5414 may be from any applicable source including data input or output from a component, step, or module shown in FIGS. 1A, 1B, 2, 3, 4A, and/or 4B. The known outcomes 5418 may be included for machine learning models generated based on supervised or semi-supervised training. An unsupervised machine learning model may not be trained using known outcomes 5418. Known outcomes 5418 may include known or desired outputs for future inputs similar to or in the same category as stage inputs 5414 that do not have corresponding known outputs.

The training data 5412 and a training algorithm 5420 may be provided to a training component 5430 that may apply the training data 5412 to the training algorithm 5420 to generate a machine learning model. According to an implementation, the training component 5430 may be provided comparison results 5416 that compare a previous output of the corresponding machine learning model to apply the previous result to re-train the machine learning model. The comparison results 5416 may be used by the training component 5430 to update the corresponding machine learning model. The training algorithm 5420 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

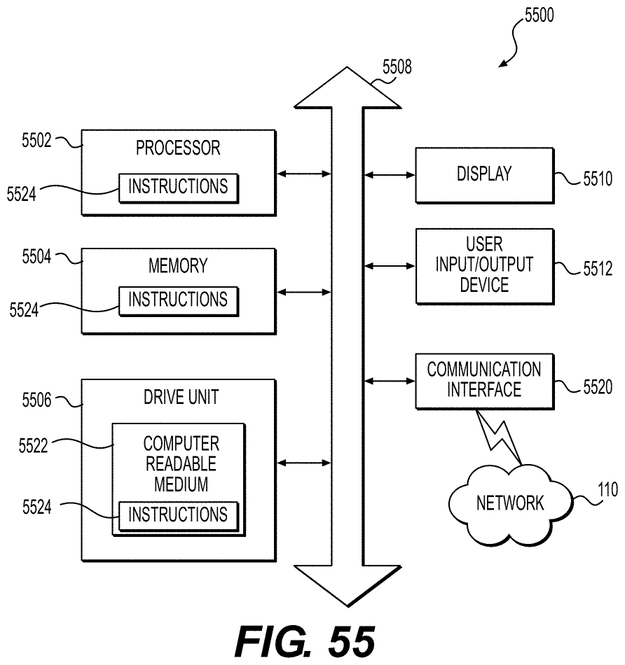

FIG. 55 is a simplified functional block diagram of a computer system 5500 that may be configured as a device for executing the techniques disclosed herein, according to exemplary embodiments of the present disclosure. FIG. 55 is a simplified functional block diagram of a computer system that may generate features, statistics, analysis and/or another system according to exemplary embodiments of the present disclosure. In various embodiments, any of the systems (e.g., computer system 5500) disclosed herein may be an assembly of hardware including, for example, a data communication interface 5520 for packet data communication. The computer system 5500 also may include a central processing unit (“CPU”) 5502, in the form of one or more processors, for executing program instructions 5524. The computer system 5500 may include an internal communication bus 5508, and a storage unit 5506 (such as ROM, HDD, SDD, etc.) that may store data on a computer readable medium 5522, although the computer system 5500 may receive programming and data via network communications (e.g., over network 110). The computer system 5500 may also have a memory 5504 (such as RAM) storing instructions 5524 for executing techniques presented herein, although the instructions 5524 may be stored temporarily or permanently within other modules of computer system 5500 (e.g., processor 5502 and/or computer readable medium 5522). The computer system 5500 also may include input and output ports 5512 and/or a display 5510 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the presently disclosed methods, devices, and systems are described with exemplary reference to transmitting data, it should be appreciated that the presently disclosed embodiments may be applicable to any environment, such as a desktop or laptop computer, a mobile device, a wearable device, an application, or the like. Also, the presently disclosed embodiments may be applicable to any type of Internet protocol.

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

Aspects of the present disclosure relate to signal based feature analysis. In one aspect, the present disclosure is directed to a method including receiving distinct electrical signals generated based on a body part, generating a plurality of extracted features based on the distinct electrical signals, and identify clinically relevant features from the plurality of extracted features, wherein the clinically relevant features meet a threshold determined based on a clinical outcome.

The method may also include applying the clinically relevant features to determine a clinical outcome result, wherein the clinical outcome result is one of a diagnosis or a treatment plan. The distinct electrical signals may be generated based on a body electrical signal generated by the body part. The distinct electrical signals may be generated based on a movement of the body part. The distinct electrical signals may be generated based on a property of the body part. The plurality of extracted features may be based on one or more of amplitude features, zero crossing rate, standard deviation, variance, root mean square, kurtosis, frequency, bandpower, or skew. The distinct electrical signals may be generated by a wearable device comprising sensors, wherein the wearable device may be configured to output a mixed signal and/or wherein a signal separation module extracts the extracted features from the mixed signal.

For example, the signal separation module may apply one or more of blind signal separation, blind source separation, discrete transform, Fourier transform, integral transform, two-sided Laplace transform, Mellin transform, Hartley transform, Short-time Fourier transform (or short-term Fourier transform) (STFT), rectangular mask short-time Fourier transform, Chirplet transform, Fractional Fourier transform (FRFT), Hankel transform, Fourier-Bros-Iagolnitzer transform, or linear canonical transform to extract the extracted features from the mixed signal. A random forest algorithm may be used to score the extracted features. The threshold may be a random forest threshold and extracted features having a random forest score at or above the random forest threshold may be identified as clinically relevant features. The threshold may be a reliability threshold and extracted features having a reliability score at or above a reliability threshold may be identified as clinically relevant features. The reliability score may be based on one or more of a spearman correlation, intraclass correlation (ICC), covariance (CV), area under a curve (AUC), clustering, or Z score.

In another aspect, the present disclosure is direct to a system including a wearable device including a plurality of sensors, a processor, a computer-readable data storage device storing instructions that, when executed by the processor, cause the system to obtain electrical activity information of a subject from the wearable device, the electrical activity detected by the plurality of sensors, and identify clinically relevant features based on the electrical activity information.

The system may be further configured to classify the clinically relevant features as one or more maladies, determine a disease of the subject based on the one or more maladies, determine a scope of the disease and/or determine a treatment plan based on the scope of the disease. The plurality of sensors may include an electroencephalography (EEG) sensor, an electrooculography (EOG) sensor, an electromyography (EMG) sensor, an image sensor, and/or an eye-tracking sensor. The clinically relevant features may be identified using a machine-learning algorithm.

What is claimed is:

1. A method comprising:
receiving distinct electrical signals generated based on a body part;
generating a plurality of extracted features based on the distinct electrical signals; and
identify clinically relevant features from the plurality of extracted features, wherein the clinically relevant features meet a threshold determined based on a clinical outcome.

2. The method of claim 1, further comprising, applying the clinically relevant features to determine a clinical outcome result.

3. The method of claim 2, wherein the clinical outcome result is one of a diagnosis or a treatment plan.

4. The method of claim 1, wherein the distinct electrical signals are generated based on a body electrical signal generated by the body part.

5. The method of claim 1, wherein the distinct electrical signals are generated based on a movement of the body part.

6. The method of claim 1, wherein the distinct electrical signals are generated based on a property of the body part.

7. The method of claim 1, wherein the plurality of extracted features are based on one or more of amplitude features, zero crossing rate, standard deviation, variance, root mean square, kurtosis, frequency, bandpower, or skew.

8. The method of claim 1, wherein the distinct electrical signals are generated by a wearable device comprising sensors.

9. The method of claim 8, wherein the wearable device is configured to output a mixed signal.

10. The method of claim 9, wherein a signal separation module extracts the extracted features from the mixed signal.

11. The method of claim 10, wherein the signal separation module applies one or more of blind signal separation, blind source separation, discrete transform, Fourier transform, integral transform, two-sided Laplace transform, Mellin transform, Hartley transform, Short-time Fourier transform (or short-term Fourier transform) (STFT), rectangular mask short-time Fourier transform, Chirplet transform, Fractional Fourier transform (FRFT), Hankel transform, Fourier-Bros-Iagolnitzer transform, or linear canonical transform to extract the extracted features from the mixed signal.

12. The method of claim 1, wherein a random forest algorithm is used to score the extracted features.

13. The method of claim 12, wherein the threshold is a random forest threshold and wherein extracted features having a random forest score at or above the random forest threshold are identified as clinically relevant features.

14. The method of claim 1, wherein the threshold is a reliability threshold and wherein extracted features having a reliability score at or above a reliability threshold are identified as clinically relevant features.

15. The method of claim 14, wherein the reliability score is based on one or more of a spearman correlation, intraclass correlation (ICC), covariance (CV), area under a curve (AUC), clustering, or Z score.

16. A system comprising:
a wearable device including a plurality of sensors;
a processor;
a computer-readable data storage device storing instructions that, when executed by the processor, cause the system to:
obtain electrical activity information of a subject from the wearable device, the electrical activity detected by the plurality of sensors; and
identify clinically relevant features based on the electrical activity information.

17. The system of claim 16, further configured to classify the clinically relevant features as one or more maladies.

18. The system of claim 17, further configured to determine a disease of the subject based on the one or more maladies.

19. The system of claim 18, wherein the system is further configured to;
determine a scope of the disease; and
determine a treatment plan based on the scope of the disease.

20. The system of claim 16, wherein the plurality of sensors comprise an electroencephalography (EEG) sensor.

21. The system of claim 16, wherein the plurality of sensors comprise an electrooculography (EOG) sensor.

22. The system of claim 16, wherein the plurality of sensors comprise an electromyography (EMG) sensor.

23. The system of claim 16, wherein the plurality of sensors comprises an image sensor.

24. The system of claim 16, wherein the plurality of sensors comprises an eye-tracking sensor.

25. The system of claim 16, wherein the clinically relevant features are identified using a machine-learning algorithm.
